(19) United States
(12) Patent Application Publication
(10) Pub. No.: US 2022/0267853 A1
(43) Pub. Date: Aug. 25, 2022

(54) LEUKAEMIC STEM CELLS

(71) Applicant: OXFORD UNIVERSITY INNOVATION LIMITED, Oxford (GB)

(72) Inventors: Paresh Vyas, Oxford (GB); Zahra Aboukhalil, Oxford (GB); Bilyana Stoilova, Oxford (GB); Dimitris Karamitros, Oxford (GB)

(21) Appl. No.: 16/768,611

(22) PCT Filed: Nov. 30, 2018
(86) PCT No.: PCT/GB2018/053485

§ 371 Date: May 29, 2020

(30) Foreign Application Priority Data
Dec. 1, 2017 (GB) 1720077.5

(51) Int. Cl. C12Q 1/6886; G16B 20/20

(52) U.S. Cl. CPC C12Q 1/6886; G16B 20/20; C12Q 2600/136; C12Q 2600/118; C12Q 2600/106; C12Q 2600/158

(57) ABSTRACT

The present invention is directed to leukaemic stem cells, in particular methods for detecting leukaemic stem cells, use of said leukaemic stem cells in the diagnosis of myeloid leukaemia, methods of treatment, and associated kits and compositions.

The present invention relates to myeloid leukaemia, and in particular leukaemic stem cells finding utility in diagnosis or prognosis thereof.

Human Acute Myeloid Leukaemia (AML) is an aggressive cancer of white blood cells and is the most common adult acute leukaemia. In more detail, AML is a cancer of the myeloid line of blood cells. It is characterized by the rapid growth of an abnormal white blood cell population. Approximately 80% of AML patients are over the age of 60 and the overall survival of this patient group lies at only approximately 5%.

AML can be classified into several subgroups. By way of example, classification according to the World Health Organization (WHO) criteria is based on examination of bone marrow aspirate or a blood sample via light microscopy. Alternatively, bone marrow or blood may be tested for chromosomal translocations by routine cytogenetic methods or fluorescent in situ hybridisation (FISH), and for specific genetic mutations (such as mutations in the FLT3, NPM1 and CEBPA genes) may be detected by polymerase chain reaction (PCR). Immunophenotyping is another method that may be used to identify the AML subtype, which involves detection of cell surface and cytoplasmic markers using flow cytometry.

Flow cytometry is a technique for counting and examining microscopic particles such as cells by suspending them in a stream of fluid and capturing the light that emerges from each cell as it passes through a laser beam. Cell surface molecules often referred to as “cluster of differentiation” (CD) molecules may be exploited in flow cytometry to characterise cell populations. For example, in fluorescence-activated cell sorting, a diagnostic antibody (labelled with a fluorophore) is employed, which binds to a surface molecule (e.g. a CD molecule) present on and characteristic of the cell population in question. Thereafter, the fluorophore (attached to the antibody) is activated by a laser beam and the fluorescence signal detected by the flow cytometer. In this manner, fluorescently-labelled antibodies can be used to detect and sort cells displaying a specific CD molecule (or set of CD molecules).

Current AML therapies typically involve induction chemotherapy followed by post-induction therapy. The goal of induction chemotherapy is to reduce the amount of leukaemic cells to less than 5% of all the nucleated cells in a bone marrow sample. Regrettably, this level of reduction of leukaemic cells is not enough to prevent disease recurrence (i.e. relapse) and almost all patients relapse without post-induction therapy. Post-induction therapy typically involves further cycles of chemotherapy, and in some cases, a hematopoietic stem cell transplant that aims to eliminate minimal residual disease (MRD). MRD is the population of leukaemic cells that is recaltricant to therapy. It is thought that this population of cells contains a sub-population of cells termed a leukaemic stem cell (LSC) population that is largely quiescent and serves to sustain disease.

Current methods used to detect MRD include real time quantitative PCR (RQ-PCR) or by multi-parameter flow cytometry (MFC). However, RQ-PCR based MRD assessment is not possible in approximately half of patients with AML. In addition, and despite recent technical developments, there is still a lack of a validated MFC methodology demonstrating clinical utility—current sensitivity levels of MFC are at least 1 log below real time that of RQ-PCR assays.

Typically, AML is preceded by chronic phase (CP) chronic myeloid leukaemia (CML) and/or myelodysplastic syndromes (MDS). AML is considered the fully malignant state with 50% of MDS patients proceeding to the AML stage.

CP-CML, a clonal myeloproliferative disease, requires the constitutively active tyrosine kinase BCR-ABL. The majority of CP-CML patients achieve a durable complete cytogenetic response with tyrosine kinase inhibitors (TKIs; e.g. imatinib, dasatinib, nilotinib). However, in the first few years after diagnosis, 1-1.5% of CP-CML patients per annum progress to a more aggressive acute leukaemia, blast phase (BP)-CML. The rate of progression of CP-CML to BP-CML falls sharply when a major molecular response to TKI therapy is obtained. Less than 10% of patients present with de novo BP-CML and two-thirds of these BP-CML patients have a myeloid immunophenotype. Response to TKIs in BP-CML is short-lived, and median survival following diagnosis of BP-CML is 6.5-11 months, with many patients developing additional mutations within the BCR-ABL kinase domain, leading to TKI resistance and rapid disease progression. Indeed BP-CML is considered a form of AML.

There have been limited studies of LSC populations in myeloid BP-CML. BP-CML is a serious unmet clinical need with poor outcomes and 5-year survival rates <20% with the only aggressive, curative option: conventional chemotherapy followed by allogeneic stem cell transplant.

The present invention solves one or more of the above-mentioned problems.

The present inventors have found that one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 advantageously allow sensitive identification of myeloid precursor cells and related leukaemic stem cells, and can be utilised for diagnosing myeloid leukaemia.

Genomic DNA sequences for each of the above-referenced genes are available from GenBank and are listed in the sequence listing herein as SEQ ID NOs:1-97 (see table). Expression of these genes may be detected by any means, such as by detecting and preferably quantifying mRNA expressed from said genes (e.g. by way of converting the mRNA into cDNA).

cDNA sequences corresponding to the genes of the invention are provided in the sequence listing herein and are also obtainable from NCBI GenBank (see the accession numbers disclosed herein). In one embodiment the methods of the invention comprise the detection of said cDNA, which corresponds to mRNA expressed from the genes. The skilled person understands that the cDNA sequences provided herein may be equivalent to the RNA except for the presence of the base “T” rather than “U”.

The methods of the invention may comprise detecting expression of a nucleic acid sequence having at least 80% (such at least 85%, 90%, 95%, 98%, 99% or 100%) sequence identity to a nucleic acid sequence provided herein, or a fragment or derivative thereof. Preferably a nucleic acid sequence having 100% sequence identity to a nucleic acid sequence provided herein.

The methods of the invention may comprise detecting an amino acid sequence translated from a gene of the invention, such as an amino acid sequence translated from a nucleic acid sequence having at least 80% (such at least 85%, 90%, 95%, 98%, 99% or 100%) sequence identity to a nucleic acid sequence provided herein, or a fragment or derivative thereof. Preferably a nucleic acid sequence having 100% sequence identity to a nucleic acid sequence provided herein.

The methods of the invention may comprise detecting expression of an amino acid having at least 80% (such at least 85%, 90%, 95%, 98%, 99% or 100%) sequence identity to an amino acid sequence provided herein, or a fragment or derivative thereof. Preferably an amino acid sequence having 100% sequence identity to an amino acid sequence provided herein.

Thus the invention provides in one aspect a gene expression profile comprising (or consisting of) one or more of MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4. Advantageously, changes in expression of said genes correlates with the presence or absence of myeloid leukaemia and/or leukaemic stem cells. The invention provides use of said gene expression profile for identifying the presence or absence of LSCs or the presence of myeloid leukaemia (e.g. AML).

Thus the invention provides in one aspect a gene expression profile comprising (or consisting of) one or more of MS4A2, MLNR, TIGIT, CNGA1, MME, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and ILDR1. Advantageously, changes in expression of said genes facilitates identification of myeloid precursor cells (e.g. non-LSC myeloid precursor cells).

In some embodiments the method of the invention comprise detecting expression of combinations of the genes described herein to detect the type of myeloid precursor cell as well as whether or not said cell is an LSC.

The term “one or more” when used in the context of a gene described herein may mean at least 2, 3, 4, 5, 6, 7, 8, 9, 10, 11, 12, 13, 14, 15, 16, 17, 18, 19, 20, 21, 22, 23 or 24 of the genes. Suitably, the term “one of more” in this context may mean all of the genes.

In one aspect the invention provides a method for identifying an LSC in a sample, said method comprising:
a. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4 in the sample:
b. comparing the detected expression of said one or more genes to expression of said one or more genes in a reference standard; and
c. identifying an LSC in said sample based on said comparison.

In one aspect the invention provides a method for identifying a leukaemic stem cell (LSC) or a myeloid precursor cell in a sample, said method comprising:

a. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample; and
b. identifying the presence of an LSC or myeloid precursor (respectively) in said sample based when expression of the gene is detected; or identifying the absence of an LSC or myeloid precursor (respectively) in said sample based when expression of the gene is not detected).

In one aspect the invention provides a method for identifying a myeloid precursor cell in a sample, said method comprising:
a. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4 in the sample;
b. comparing the detected expression of said one or more genes to expression of said one or more genes in a reference standard; and
c. identifying a myeloid precursor cell in said sample based on said comparison.

Preferably the myeloid precursor is an LSC.

Alternatively or additionally, in one aspect the invention provides a method for identifying a myeloid precursor cell in a sample, said method comprising:
a. detecting expression of one or more genes selected from: MS4A2, MLNR, TIGIT, CNGA1, MME, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and ILDR1 in the sample;
b. comparing the detected expression of said one or more genes to expression of said one or more genes in a reference standard; and
c. identifying a myeloid precursor cell in said sample based on said comparison.

In a related aspect there is provided a method for diagnosing myeloid leukaemia comprising detecting the presence or absence of a leukaemic stem cell (LSC) in a sample, said method comprising:
a. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4 in the sample;
b. comparing the detected expression of said one or more genes to expression of said one or more genes in a reference standard; and
c. identifying the presence or absence of an LSC in said sample based on said comparison;
wherein myeloid leukaemia is diagnosed when said LSC is present in the sample; and
wherein myeloid leukaemia is not diagnosed when said LSC is absent from the sample.

In a related aspect there is provided a method for diagnosing myeloid leukaemia comprising detecting the presence or absence of a leukaemic stem cell (LSC) in a sample, said method comprising:
a. detecting expression of one or more genes selected from: MS4A2, MLNR, TIGIT, CNGA1, MME, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and ILDR1 in the sample;
b. comparing the detected expression of said one or more genes to expression of said one or more genes in a reference standard; and
c. identifying the presence or absence of an LSC in said sample based on said comparison;
wherein myeloid leukaemia is diagnosed when said LSC is present in the sample; and
wherein myeloid leukaemia is not diagnosed when said LSC is absent from the sample.

In one embodiment a method of the invention comprises detecting expression of MS4A2.

In one embodiment a method of the invention comprises detecting expression of one or more of MLNR, TIGIT, and CNGA1.

In one embodiment a method of the invention comprises detecting expression of MME.

In one embodiment a method of the invention comprises detecting expression of one or more of SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and ILDR1.

The detected gene expression in the reference standard may have been obtained (e.g. quantified) previously to a method of the invention. The expression level of the genes described herein is suitably known in said reference standard. A reference standard is preferably from the same source as the sample referred to in a method of the invention. For example, both the sample and reference standard may be from bone marrow, or both may be from blood.

In one embodiment increased expression (upregulation) of MS4A2 in a sample when compared to the expression in a non-CMP reference standard identifies the presence of a CMP cell in said sample. Analogously no difference in expression of MS4A2 in a sample when compared to the expression in a CMP myeloid precursor cell reference standard identifies the presence of a CMP cell in said sample (the skilled person will also appreciate that detection of decreased expression levels may indicate that the sample does not contain a CMP cell).

A “non-CMP reference standard” may comprise haematopoietic stem cells and/or myeloid precursors cells, such as MPP, LMPP, GMP, MEP and/or MLP cells.

In one embodiment increased expression (upregulation) of one or more of MLNR, TIGIT and/or CNGA1 (preferably MLNR, TIGIT and CNGA) in a sample when compared to the expression in a non-MPP reference standard identifies the presence of a MPP cell in said sample. Analogously no difference in expression of one or more of MLNR, TIGIT and/or CNGA1 (preferably MLNR, TIGIT and CNGA) in a sample when compared to the expression in a MPP myeloid precursor cell reference standard identifies the presence of a MPP cell in said sample (the skilled person will also appreciate that detection of decreased expression levels may indicate that the sample does not contain a MPP cell).

A “non-MMP reference standard” may comprise haematopoietic stem cells and/or myeloid precursors cells, such as CMP, LMPP, GMP, MEP and/or MLP cells.

In one embodiment increased expression (upregulation) of MME in a sample when compared to the expression in a non-LMPP reference standard identifies the presence of a LMPP cell in said sample. Analogously no difference in expression of MME in a sample when compared to the expression in a LMPP myeloid precursor cell reference standard identifies the presence of a LMPP cell in said sample (the skilled person will also appreciate that detection of decreased expression levels may indicate that the sample does not contain a LMPP cell).

A “non-LMMP reference standard” does not comprise a MLP cell, but may comprise haematopoietic stem cells and/or myeloid precursor cells, such as CMP, MPP, GMP, and/or MEP cells.

In one embodiment increased expression (upregulation) of one or more of SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and/or ILDR1 (preferably SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and ILDR1) in a sample when compared to the expression in a non-GMP reference standard identifies the presence of a GMP cell in said sample. Analogously no expression of one or more of SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and/or ILDR1 (preferably SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and ILDR1) in a sample when compared to the expression in a GMP myeloid precursor cell reference standard identifies the presence of a GMP cell in said sample (the skilled person will also appreciate that detection of decreased expression levels may indicate that the sample does not contain a GMP cell).

A “non-GMP reference standard” may comprise haematopoietic stem cells and/or myeloid precursors cells, such as CMP, MPP, LMPP, MEP and/or MLP cells.

A “CMP reference standard”, “MPP reference standard”, “LMPP reference standard” and “GMP reference standard” preferably only includes CMP, MPP, LMPP, and GMP cells respectively, and in one embodiment does not contain LSCs or is a non-myeloid leukaemia reference standard.

The terms “myeloid precursor cell” and “myeloid progenitor cell” are used synonymously herein.

The term “no expression” used herein encompasses “substantially no expression”.

In one embodiment methods of the invention comprise detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4.

The detected expression of these genes may be compared to a LMPP reference standard or a GMP reference standard, wherein the LMPP and GMP cells (respectively) are non-LSCs and/or where the reference standard is a non-myeloid leukaemia (e.g. non-AML) reference standard.

In one embodiment, when compared to such a reference standard increased expression (upregulation) of one or more of IFITM1, CMTM6, CD55, SLC35F5, AQP11, PCDHB9, RHOA, SAMD8, TAPT1, LEPROT, IL6ST, EMP3, CD83, LPAR6, PIEZO2, IL1RAP, LPAR4, PKN2, TMCO3, VAMP7, PTPRC, TFRC, PDIA3, SLC9B1, CD99, TEX10, CNGB1, PDGFA, SLC12A8, SLC2A14, RNF19B, CD36, HSPA5, ITGAX, SLC2A3, IL18R1, CCDC47, SORCS1, CLIC4, DERL1, VAMP2, AIMP1, UBR4, ATP6AP2, CD46, SPCS1, ITM2B, TMEM50A, FZD4, and/or SHISA9 identifies the presence of an LSC or the presence of myeloid leukaemia (e.g. AML).

In one embodiment when compared to such a reference standard increased expression (upregulation) of one or more of IFITM1, CMTM6, CD55, SLC35F5, AQP11, PCDHB9, RHOA, SAMD8, TAPT1, LEPROT, IL6ST, EMP3, CD83, LPAR6, PIEZO2, IL1RAP, LPAR4, PKN2, TMCO3, VAMP7, PTPRC, TFRC, PDIA3, SLC9B1, CD99, TEX10, CNGB1, PDGFA, SLC12A8, SLC2A14, RNF19B, CD36, HSPA5, ITGAX, SLC2A3, IL18R1, CCDC47, SORCS1, and/or CLIC4 identifies the presence of an LSC or the presence of myeloid leukaemia (e.g. AML).

Preferably when compared to such a reference standard increased expression (upregulation) of one or more of IFITM1, AQP11, IL6ST, CD83, LPAR6, and/or LPAR4 identifies the presence of an LSC or the presence of myeloid leukaemia (e.g. AML). More preferably, when compared to such a reference standard increased expression (upregulation) of one or more of AQP11, IFITM1, and/or LPAR6 identifies the presence of an LSC or the presence of myeloid leukaemia (e.g. AML).

Preferably the LSC is a LMPP LSC or GMP LSC.

The skilled person will appreciate that in some embodiments no change in expression of said genes when compared to a LMPP reference standard or a GMP reference standard or decreased expression (deregulation) of said genes may indicate the absence of an LSC or the absence of myeloid leukaemia (e.g. AML). Likewise, the skilled person will appreciate that when the reference standard is a LMPP LSC or GMP LSC reference standard, no change in expression (or increased expression) may indicate the presence of an LSC or the presence of myeloid leukaemia (e.g. AML), while a decrease in expression may indicate the absence of an LSC or the absence of myeloid leukaemia (e.g. AML).

In one embodiment, when compared to such a reference standard decreased expression (downregulation) of one or more of MME, CNTNAP2, PIGO, SHH, TMEM231, ABCA13, NFASC, MCOLN2, SERPINE2, ILDR1, GPR63, CCR7, ATG9B, LRP1, SPN, PILRB, JAM2, NDUFB1, GYPE, SLC35F6, GLG1, SMIM24, SLC24A2, and/or RAB11FIP3 identifies the presence of an LSC or the presence of myeloid leukaemia (e.g. AML).

Preferably when compared to such a reference standard decreased expression (downregulation) of one or more of MME, PIGO, SHH, ABCA13, SERPINE2, CCR7, ATG9B, and/or GYPE identifies the presence of an LSC or the presence of myeloid leukaemia (e.g. AML). More preferably when compared to such a reference standard decreased expression (downregulation) of one or more of MME, and/or SHH identifies the presence of an LSC or the presence of myeloid leukaemia (e.g. AML).

The present methods may include the use of genes that are upregulated and those that are downregulated in identifying an LSC or other cell of the invention.

The skilled person will appreciate that in some embodiments no change in expression of said genes when compared to a LMPP reference standard or a GMP reference standard or increased expression (upregulation) of said genes may indicate the absence of an LSC or the absence of myeloid leukaemia (e.g. AML). Likewise, the skilled person will appreciate that when the reference standard is a LMPP LSC or GMP LSC reference standard, no change in expression (or a decrease in expression) may indicate the presence of an LSC or the presence of myeloid leukaemia (e.g. AML), while an increase in expression may indicate the absence of an LSC or the absence of myeloid leukaemia (e.g. AML).

Further distinctions in gene expression levels between LMPPs and GMPs from myeloid leukaemia reference standards (and optionally the counterparts from non-myeloid leukaemia reference standards) can be made by reference to the expression data of FIG. 11.

Further distinctions in gene expression levels between LMPP LSCs and GMP LSCs (and optionally their non-LSC counterparts) can be made by reference to the expression data of FIG. 11.

The invention also provides a kit comprising means for detecting expression of one or more genes of the invention. In one embodiment, the means for detecting gene expression is a probe for use in quantitative RT-PCT (such as a Taqman probe). Primers or antibodies may also be used to measure gene expression levels. As discussed above, methods for assessing gene expression levels are conventional techniques known to those skilled in the art and a skilled person would readily be able to design and/or select suitable detection agents for use in inter alia the kits of the present invention. In one embodiment, the kit may further comprise instructions explaining how to use the means for detecting expression of one or more genes in a method of the invention.

The terms “detecting expression” and “detected expression” encompass detecting both negative (e.g. no expression) and positive expression (e.g. expression). In one embodiment the expression is positive expression.

Detection may be carried out by any means known to the person skilled in the art. For example, detection may be at the level of transcription or translation. For instance, mRNA of a target gene can be detected and quantified by e.g. Northern blotting or by quantitative reverse transcription PCR (RT-PCR). Single cell gene expression analysis may also be performed using commercially available systems (e.g. Fluidigm Dynamic Array). Alternatively, or in addition, gene expression levels can be determined by analysing protein levels e.g. by using Western blotting techniques such as ELISA-based assays. Thus, in one embodiment, gene expression levels are determined by measuring the mRNA/cDNA levels of the genes belonging to the gene expression profile of the present invention. In another embodiment, gene expression levels are determined by measuring the protein levels produced by the genes belonging to the gene expression profile of the present invention. Methods suitable for establishing a baseline or reference value for comparing gene expression levels are conventional techniques known to those skilled in the art.

Increased expression (upregulation) refers to a detected gene expression level of greater than 0 fold relative to a reference standard, e.g. greater than 1-fold. In one embodiment increased expression means greater than 1.25-fold to about 10-fold or more gene expression relative to a reference standard. In some embodiments, increased expression means greater than at least about 1.1-fold, 1.2-fold, 1.25-fold, 1.5-fold, 1.75-fold, 2-fold, 4-fold, 5-fold, 10-fold, 15-fold, 20-fold, 25-fold, 30-fold, 35-fold, 40-fold, 50-fold, 75-fold, 100-fold, 150-fold, 200-fold, or at least about 300-fold gene expression when compared to a reference standard.

Decreased expression (downregulation) refers to a detected gene expression level of less than 0 fold relative to a reference standard, e.g. less than −1-fold. In one embodiment decreased expression means less than −1.25-fold to about −10-fold or more gene expression relative to a reference standard. In some embodiments, decreased expression means less than at least about −1.1-fold, −1.2-fold, −1.25-fold, −1.5-fold, −1.75-fold, −2-fold, −4-fold, −5-fold, −10-fold, −15-fold, −20-fold, 25-fold, −30-fold, −35-fold, −40-fold, −50-fold, −75-fold, −100-fold, −150-fold, −200-fold, or at least about −300-fold gene expression when compared to a reference standard.

The fold change difference can be in absolute terms (e.g. CPM: counts per million) or Log 2CPM (a standard measure in the field) of the gene expression level in a sample. Preferably the fold change is Log 2 fold change.

In one embodiment said fold-change is measured/is determined by RNA sequencing (RNA-Seq), e.g. in toto.

A cell comprising a cell surface polypeptide marker phenotype CD34⁺; CD45RA⁻; CD123⁺; and CD38⁺ is also referred to herein as a “CMP cell”.

A cell comprising a cell surface polypeptide marker phenotype CD34⁺, CD45RA⁻; CD90⁻; and CD38⁻ is also referred to herein as a “MPP cell”.

A cell comprising a cell surface polypeptide marker phenotype CD34⁺; CD45RA⁺; CD123⁺; and CD38⁻ is also referred to herein as a “LMPP cell”.

A cell comprising a cell surface polypeptide marker phenotype CD34⁺; CD45RA⁺; CD123⁺; and CD38⁺ is also referred to herein as a “GMP cell”.

A cell comprising a cell surface polypeptide marker phenotype CD34⁺; CD45RA⁻; CD123⁻; and CD38⁺ is also referred to herein as a “MEP cell”.

A cell comprising a cell surface polypeptide marker phenotype CD34⁺; CD45RA⁺; CD90⁻; CD38⁻; and CD10⁺ is also referred to herein as a “MLP cell”.

A cell surface polypeptide marker phenotype CD34⁺; CD45RA⁻; CD123⁻; CD90⁺; and CD38⁻ is also referred to herein as a “haematopoietic stem cell” (HSC).

In one embodiment the above-mentioned cells are leukaemic stem cells.

In one aspect the present invention is directed to leukaemic stem cells (LSCs). Said LSCs preferably express one or more of MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4. More preferably one or more of IFITM1, CMTM6, CD55, SLC35F5, AQP11, PCDHB9, RHOA, SAMD8, TAPT1, LEPROT, IL6ST, EMP3, CD83, LPAR6, PIEZO2, IL1RAP, LPAR4, PKN2, TMCO3, VAMP7, PTPRC, TFRC, PDIA3, SLC9B1, CD99, TEX10, CNGB1, PDGFA, SLC12A8, SLC2A14, RNF19B, CD36, HSPA5, ITGAX, SLC2A3, IL18R1, CCDC47, SORCS1, CLIC4, DERL1, VAMP2, AIMP1, UBR4, ATP6AP2, CD46, SPCS1, ITM2B, TMEM50A, FZD4, and SHISA9. Said LSCs may express one or more genes selected from: MS4A2, MLNR, TIGIT, CNGA1, MME, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and ILDR1.

In one aspect the LSCs comprise the cell surface polypeptide marker phenotype: CD34⁺; CD45RA⁻; CD123+; and CD38⁺; wherein (+) indicates the presence and (−) indicates the absence of said cell surface polypeptide markers. In another aspect the present invention is directed to LSCs comprising the cell surface polypeptide marker phenotype CD34⁺, CD45RA⁻; CD90⁻; and CD38⁻. The invention is also related to uses of said LSCs.

The term “leukaemic stem cell” as used herein refers to a cell that is capable of self-renewal, proliferation, and/or differentiation. A “leukaemic stem cell” may be identifiable by a serial transplantation assay (e.g. in addition to the methods of the invention).

A serial transplantation assay may comprise:
administering (e.g. via IV) a cell to a first immuno-deficient murine recipient; determining whether said cell engrafts in the recipient;
isolating an engrafted cell from the primary recipient;
administering the cell to a second immuno-deficient murine recipient; and determining whether the cell engrafts in the secondary recipient;
wherein when the cell engrafts in a secondary recipient the cell is identified as a leukaemic stem cell; and wherein when the cell does not engraft in a secondary recipient the cell is identified as not being a leukaemic stem cell.

Thus, in one embodiment a LSC engrafts in a secondary recipient when tested in a serial transplantation assay.

The LSCs referred to herein preferably have one or more of: chromosome 17p loss (e.g. loss of 17p13), isochromosome 17q, a BCR-ABL fusion gene or combinations thereof. In one embodiment the LSCs referred to herein have two or more of 17p loss (e.g. loss of 17p13), isochromosome 17q, and a BCR-ABL fusion gene. Preferably the LSCs have 17p loss (e.g. loss of 17p13), isochromosome 17q, and a BCR-ABL fusion gene.

The LSCs preferably have one or more of the karyotypic abnormalities detailed in Table 1(b) herein, such as one or more of: t(9:22) (q34; 11), del(16) (q22), del(16) (q22q23), i(17) (q10), i(17) (?q10), 46, idem, del(7)(p11)/46, XY, 46, idem, and del(17)(p1?3). Preferably a LSC has two or more, three or more, four or more, five or more, six or more, seven or more, or all of the above-referenced karyotypic abnormalities.

A method of the invention may comprise determining whether one of the above-mentioned properties is present in a detected cell. Additionally or alternatively, a method of the invention may further comprise validating that the detected cell is a LSC by way of the serial transplantation assay described above, or by way of a comparable assay known to the skilled person.

In one aspect the invention provides a method for diagnosing myeloid leukaemia, said method comprising:
detecting the presence or absence of a leukaemic stem cell (LSC) in a sample; wherein the LSC comprises a cell surface polypeptide marker phenotype: CD34⁺; CD45RA⁻; CD123⁺; and CD38⁺; wherein (+) indicates the presence and (−) indicates the absence of said cell surface polypeptide markers.

In one aspect the invention provides a method for diagnosing myeloid leukaemia, said method comprising:
detecting the presence or absence of a leukaemic stem cell (LSC) in a sample; wherein the LSC comprises a cell surface polypeptide marker phenotype: CD34⁺, CD45RA⁻; CD90⁻; and CD38⁻; wherein (+) indicates the presence and (−) indicates the absence of said cell surface polypeptide markers.

In embodiments when an LSC is present in the sample myeloid leukaemia is diagnosed. In embodiments when an LSC is not present in the sample myeloid leukaemia is not diagnosed.

The LSCs can also be used to determine prognosis in myeloid leukaemia, thus in one aspect there is provided a method comprising: detecting the presence or absence of a leukaemic stem cell (LSC) in a sample; wherein the LSC comprises a cell surface polypeptide marker phenotype: CD34⁺; CD45RA⁻; CD123⁺; and CD38⁺. In another aspect there is provided a method comprising: detecting the presence or absence of a leukaemic stem cell (LSC) in a sample; wherein the LSC comprises a cell surface polypeptide marker phenotype: CD34⁺, CD45RA⁻; CD90⁻; and CD38⁻. A poor prognosis is determined when said LSC is present in the sample; and a good prognosis is determined when said LSC is absent from the sample.

The present invention also provides use of a leukaemic stem cell for diagnosing myeloid leukaemia, or for determining prognosis in myeloid leukaemia, in vitro. In one embodiment the leukaemic stem cell comprises a cell surface polypeptide marker phenotype: CD34⁺; CD45RA⁻; CD123⁺; and CD38⁺. In another embodiment the leukaemic stem cell comprises a cell surface polypeptide marker phenotype: CD34⁺, CD45RA⁻; CD90⁻; and CD38⁻.

The myeloid leukaemia referred to herein may be MDS, MRD, chronic myeloid leukaemia (CML) or acute myeloid leukaemia (AML). Preferably the myeloid leukaemia is AML.

In one embodiment the leukaemia is chronic myeloid leukaemia (CML). CML can be split up into different disease phases. Chronic phase-CML (CP-CML) is the first disease phase which may be associated with constitutively active tyrosine kinase BCR-ABL. Some subjects progress to accelerated phase CML (AP-CML), and ultimately blast phase CML (BP-CML) characterised by more aggressive leukaemic symptoms, and short-term patient survival.

CP-CML is largely asymptomatic, and typically when symptoms are present, they are of a mild nature including fatigue, left side pain, joint and/or hip pain, or abdominal fullness.

AP-CML may be diagnosed when one or more of the following are present:
i. 10-19% myeloblasts in the blood or bone marrow;
ii. >20% basophils in the blood or bone marrow;
iii. Platelet count <100,000, unrelated to therapy;
iv. Platelet count >1,000,000, unresponsive to therapy;
v. Cytogenetic evolution with new abnormalities in addition to the Philadelphia chromosome; and/or
vi. Increasing splenomegaly or white blood cell count, unresponsive to therapy.

BP-CML may be diagnosed when one or more of the following are present:
i. >20% myeloblasts or lymphoblasts in the blood or bone marrow;
ii. Large clusters of blasts in the bone marrow on biopsy; and/or
iii. Development of a chloroma (solid focus of leukaemia outside the bone marrow).

In one embodiment the present invention preferably diagnoses BP-CML.

The LSCs of the invention may be isolated. For example, the LSCs may be separated from other cell types using an appropriate technique such as a flow cytometric techniques, e.g. fluorescence activated cell sorting (FACS). In a related aspect there is provided a composition comprising a LSC of the invention. The composition may be enriched in LSCs, for example said LSCs may constitute at least 70%, 75%, 80%, 85%, 90% or 95% of the total cells comprised in the composition.

Preferably, the composition comprises detecting means, wherein said detecting means facilitates detection of one or more of the cell surface polypeptide markers.

A cell surface polypeptide marker may be displayed (at least in part) on the extracellular surface of a cell. Markers of the present invention may include CD34, CD45RA, CD90, CD123, CD38, CD10, CD19, Lin, and CD33. CD34 is a heavily glycosylated, 105-120 kDa transmembrane glycoprotein expressed on hematopoietic progenitor cells, vascular endothelial cells and some fibroblasts. The CD34 cytoplasmic domain is a target for phosphorylation by activated protein kinase C, suggesting a role for CD34 in signal transduction. CD34 may also play a role in adhesion of certain antigens to endothelium. CD45R, also designated CD45 and PTPRC, has been identified as a transmembrane glycoprotein, broadly expressed among hematopoietic cells. Multiple isoforms of CD45R are distributed throughout the immune system according to cell type including CD45RA. CD45R functions as a phosphotyrosine phosphatase, a vital component for efficient tyrosine phosphorylation induction by the TCR/CD3 complex. CD90 is a 25-37 kDa heavily N-glycosylated, glycophosphatidylinositol (GPI) anchored conserved cell surface protein originally discovered as a thymocyte antigen. The CD123 antigen (also known as interleukin-3 receptor) is a molecule found on cells which helps transmit the signal of interleukin-3, a soluble cytokine important in the immune system. CD38, also known as cyclic ADP ribose hydrolase is a glycoprotein found on the surface of many immune cells (white blood cells). CD38 is thought to function in cell adhesion, signal transduction and calcium signalling. CD19 is a 95 kDa type-I transmembrane glycoprotein that belongs to the immunoglobulin superfamily. It is expressed on B cells throughout most stages of B cell differentiation and associates with CD21, CD81, and CD225 (Leu-13) forming a signal transduction complex. CD19 functions as a regulator in B cell development, activation, and differentiation. CD10 is a single pass, type II transmembrane, 100 kDa cell surface glycoprotein belonging to peptidase M13 family. CD33 is a transmembrane receptor expressed on cells of myeloid lineage.

When used in the context of cell surface polypeptide marker phenotypes herein (+) indicates the presence and (−) indicates the absence of said cell surface polypeptide markers. Any suitable detection means can be employed to determine the presence or absence of said markers. In one embodiment, the presence (+) of a marker refers to an elevation in the levels of marker in a sample above a background level. Likewise, the absence (−) of a marker refers to a reduction in the levels of a marker in a sample below a background level. In one embodiment, the elevation in the levels of marker in a sample above a background level is 1 or more (such as 2, 3, 4, 5, 6, 7, 8, 10, 15, 20, 25) fluorescence units. In one embodiment a reduction in the levels of a marker in a sample below a background level is 1 or more (such as 2, 3, 4, 5, 6, 7, 8, 10, 15, 20, 25) fluorescence units. In this regard, it would be routine for a skilled person in the art to determine the background level of marker expression in a sample.

Preferably the detection means comprises one or more antibodies that bind to a cell surface polypeptide marker. Thus, in one embodiment, said cell surface polypeptide markers may be detected by specific binding of said one or more antibodies.

The term “antibody” is used in the broadest sense and specifically covers monoclonal and polyclonal antibodies (and fragments thereof) so long as they exhibit the desired biological activity. In particular, an antibody is a protein including at least one or two, heavy (H) chain variable regions (abbreviated herein as VHC), and at least one or two light (L) chain variable regions (abbreviated herein as VLC). The VHC and VLC regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. Sequences of Proteins of Immunological Interest, Fifth Edition, U.S. Department of Health and Human Services, NIH Publication No. 91-3242, 1991, and Chothia, C. et al, J. Mol. Biol. 196:901-917, 1987). Preferably, each VHC and VLC is composed of three CDRs and four FRs, arranged from amino-terminus to carboxy-terminus in the following order FRI, CDR1, FR2, DR2, FR3, CDR3, FR4. The VHC or VLC chain of the antibody can further include all or part of a heavy or light chain constant region. In one embodiment, the antibody is a tetramer of two heavy immunoglobulin chains and two light immunoglobulin chains, wherein the heavy and light immunoglobulin chains are interconnected by, e.g., disulfide bonds. The heavy chain constant region includes three domains, CH1, CH2 and CH3. The light chain constant region is comprised of one domain, CL. The variable region of the heavy and light chains contains a binding domain that interacts with an antigen. The term “antibody” includes intact immunoglobulins of types IgA, IgG, IgE, IgD, IgM (as well as subtypes thereof), wherein the light chains of the immunoglobulin may be of types kappa or lambda. The term antibody, as used herein, also refers to a portion of an antibody that binds to one of the above-mentioned markers, e.g., a molecule in which one or more immunoglobulin chains is not full length, but which binds to a marker. Examples of binding portions encompassed within the term antibody include (i) a Fab fragment, a monovalent fragment consisting of the VLC, VHC, CL and CH1 domains; (ii) a F(ab′)2 fragment, a bivalent fragment comprising two Fab fragments linked by a disulfide bridge at the hinge region; (iii) a Fc fragment consisting of the VHC and CH1 domains; (iv) a Fv fragment consisting of the VLC and VHC domains of a single arm of an antibody, (v) a dAb fragment (Ward et al, Nature 341:544-546, 1989), which consists of a VHC domain; and (vi) an isolated complementarity determining region (CDR) having sufficient framework to bind, e.g. an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al. (1988) Science IAI-ATi-Alp; and Huston et al. (1988) Proc. Natl. Acad. ScL USA 85:5879-5883). Such single chain antibodies are also encompassed within the term antibody. These may be obtained using conventional techniques known to those skilled in the art, and the portions are screened for utility in the same manner as are intact antibodies.

Preferably an antibody as used herein comprises a heavy chain with three CDRs (CDR1, CDR2, and CDR3) and a light chain with three CDRs (CDR1, CDR2, and CDR3). More preferably an antibody as used herein comprises a VLC and VHC.

The antibodies of the present invention can be obtained using conventional techniques known to persons skilled in the art and their utility confirmed by conventional binding studies. By way of example, a simple binding assay is to incubate the cell expressing an antigen with the antibody. If the antibody is tagged with a fluorophore, the binding of the antibody to the antigen can be detected by FACS analysis.

Antibodies of the present invention can be raised in various animals including mice, rats, rabbits, goats, sheep, monkeys or horses. Blood isolated from these animals contains polyclonal antibodies—multiple antibodies that bind to the same antigen. Antigens may also be injected into chickens for generation of polyclonal antibodies in egg yolk. To obtain a monoclonal antibody that is specific for a single epitope of an antigen, antibody-secreting lymphocytes are isolated from an animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies. Methods for producing monoclonal antibodies are conventional techniques known to those skilled in the art (see e.g. Making and Using Antibodies: A Practical Handbook. GC Howard. CRC Books. 2006. ISBN 0849335280). Polyclonal and monoclonal antibodies are often purified using Protein A/G or antigen-affinity chromatography.

The detection means may comprise one or more antibodies that bind to CD34, CD45RA, CD123, CD90, and CD38. In one embodiment the detection means comprises antibodies that bind to CD34, CD45RA, and CD38. Preferably the detection means further comprises antibodies that bind to CD123 and/or CD90.

In one embodiment the presence or absence of lineage cell surface markers as described in Example 1, as well as any of CD45, CD33, CD10, and CD19 may be determined.

Therefore, the detection means may comprise antibodies that bind to said cell surface markers.

The detection means is preferably specific for a single marker. Thus, in one embodiment where the detection means is an antibody, said antibody may specifically bind to only one of CD34, CD45RA, CD123, CD90, CD38, CD45, CD33, CD10 or CD19. For example, the antibody may specifically bind to CD34 and may not bind to any of CD45RA, CD123, CD90, CD38, CD45, CD33, CD10 and CD19.

In one embodiment, the antibodies of the present invention recognise and bind to specific epitopes of the above mentioned cell surface polypeptide markers. For example, an antibody of the present invention may bind to an epitope in the N-terminal/C-terminal/mid-region domains/extracellular domains of CD34, CD45RA, CD123, CD90, CD38, lineage markers, CD45, CD33, CD10 or CD19.

The sequence of CD34, CD45RA, CD123, CD90, CD38, CD45, CD33, CD10 and CD19 are available from the NCBI website (http://www.ncbi.nlm.nih.gov/projects/genome/assembly/grc/human/index.shtml). These amino acid sequences are provided as in the sequence listing section herein.

In one embodiment, the detection means (e.g. antibodies) bind to a CD34, CD45RA, CD123, CD90, CD38, CD45, CD33, CD10 or CD19 polypeptide comprising an amino acid sequence having at least 80% (such at least 85%, 90%, 95%, 98%, 99% or 100%) sequence identity to the sequences thereof provided herein, or a fragment thereof. In one embodiment, the detection means (e.g. antibodies) bind to a CD34, CD45RA, CD123, CD90, CD38, CD45, CD33, CD10 or CD19 polypeptide comprising an amino acid sequence encoded by a nucleic acid having at least 80% (such at least 85%, 90%, 95%, 98%, 99% or 100%) sequence identity to the sequences thereof provided herein, or a fragment thereof

Conventional methods for determining nucleic acid sequence identity are discussed in more detail later in the specification.

In one embodiment, the antibodies are polyclonal and/or monoclonal antibodies.

In one embodiment, an antibody that binds to one of the above-mentioned cell surface polypeptide markers is one capable of binding that marker with sufficient affinity such that the antibody is useful as a diagnostic/and or prognostic agent. In one embodiment, the term “binds” is equivalent to “specifically binds”. An antibody that binds/specifically binds to a cell surface polypeptide marker of interest is one that binds to one of the above mentioned markers with an affinity (Kₐ) of at least 10⁴ M.

Suitable antibodies of the present invention may include one or more antibodies described in WO2015/084166, WO2012/085574, or WO2016/083777 (each of which are incorporated herein in their entirety by reference thereto). Such antibodies may include FITC or PE-Cy7 conjugated anti-CD38, PE or FITC-conjugated anti-CD45RA, PE-Cy7-conjugated or APC conjugated anti-CD123, biotin-conjugated anti-CD90, PE-Cy5 or PERCP-conjugated anti-CD34, which are available from a number of different commercial suppliers including BD Biosciences Europe ebioscience, Beckman Coulter and Pharmingen.

In a preferred embodiment, the antibody is a labelled antibody, such as a fluorescently labelled antibody. Suitable labelled compounds include conventionally known labelled compounds, such as fluorescent substances such as cyanine dyes Cy3 (registered trademark of Amersham Life Science), fluorescein isothiacyanate (FITC), allophycocyanin (APC), rhodamine, Phycoerythrin (PE), PE-Cy5 (Phycoerythrin-Cy5), PE-Cy7 (Phycoerythrin-Cy7), APC-Alexa Fluor 750, APC-eFluor 780, Pacific Blue, Horizon V450 and quantum dot, biotin-conjugated; light scattering substances such as gold particles; photo-absorptive substances such as ferrite; radioactive substances such as iodine-125; and enzymes such as peroxidase or alkali phosphatase.

In one embodiment of the invention, different antibodies are labelled respectively with mutually distinguishable labels. Labelling may be conducted by binding a labelled compound directly to each antibody. Preferably, the antibodies are labelled with different fluorescent dyes with different fluorescence wavelengths to enable easy discrimination from one another. For example a first antibody may be labelled in red (for example PE-Cy5), a second antibody in orange (for example PI, APC, R-PE) and a third antibody in green (for example Alexa488, FITC). Suitable labelling strategies are routine and known to a person skilled in the art. By way of example, the Lightening Link™ antibody labeling kit may be used (Innova Biosciences, UK).

In one embodiment an antibody for use in a method of the invention is one or more (preferably all) of: FITC-CD45RA; PE-CLL-1; PE-TIM-3; PE-CD7; PE-CD11b; PE-CD22; PE-CD56; PerCp-CY5.5-CD123; PeCy7-CD33; APC-CD38; APC-H7-CD44; BV421CD34; and V500c-CD45.

In one embodiment an antibody may be one or more selected from: FITC-CD45RA; PerCp-CY5.5-CD123; APC-CD38; and BV421CD34. Preferably each of said antibodies may be used in a method of the invention.

In some embodiments a method of the invention may employ one or more of the antibodies referred to in the Examples (see Example 1).

Methods suitable for detection of the cell surface polypeptide markers of the present invention using labelled antibodies are conventional techniques known to those skilled in the art. For example, when a fluorescent label is used, an antibody that specifically binds to a marker may be detected by observing the emitted fluorescence colour under a microscope. A fluorescent label can also be detected by irradiating a sample with an exciting light—if the label is present, fluorescence is emitted from the sample. Thus, whether a cell is positive or negative for a particular cell surface marker may be judged by using a labelled antibody specific for said marker and observing the emitted fluorescence colour under a microscope. In a preferred embodiment of the invention, fluorescence-activated cell sorting (FACS) is used for detection of the cell surface polypeptide markers/labelled antibodies of the present invention.

In a preferred embodiment FACS gating is employed to determine the cell surface marker polypeptide phenotype on a single cell of the invention.

The cells described herein are all typically lineage negative (Lin⁻). In some embodiments the cells may comprise the presence or absence of CD10 (preferably the absence of CD10). Typically said cells may be negative for CD19 and/or CD33.

In one aspect the invention provides a method for diagnosing myeloid leukaemia, said method comprising:
a. detecting the concentration of a cell in a sample, wherein the cell is identified or detected by way of a gene expression profile described herein, or wherein the cell comprises a cell surface polypeptide marker phenotype:
i. CD34⁺; CD45RA⁻; CD123⁺; and CD38⁺; or
ii. CD34⁺, CD45RA⁻; CD90⁻; and CD38⁻;
wherein (+) indicates the presence and (−) indicates the absence of said cell surface polypeptide markers;
b. comparing the concentration of the detected cell with the concentration of a cell with the same cell surface polypeptide marker phenotype in a diagnostic reference standard; and
c. identifying the presence or absence of a concentration difference;
wherein said presence or absence of a concentration difference correlates with the presence or absence of myeloid leukaemia.

The cell is a blood cell, and in one embodiment is a leukaemic stem cell. In one embodiment the method may further comprise a step of confirming that the cell in said sample is a leukaemic stem cell.

In one embodiment the detected cell is one or more or two or more selected from: a CMP cell, a MPP cell, a LMPP cell, and a GMP cell. Preferably the detected cell is three or more selected from: a CMP cell, a MPP cell, a LMPP cell, and a GMP cell.

The methods of the invention encompass comparing the concentration of the detected cell with the concentration of a cell with the same surface polypeptide marker phenotype in a diagnostic reference standard. The cell concentration in the diagnostic reference standard may have been obtained (e.g. quantified) previously to a method of the invention. The presence or absence of a concentration difference when compared to said diagnostic reference standard correlates with myeloid leukaemia.

The diagnostic reference standard is preferably from the same sample source as the sample referred to in a method of the invention. For example, both the sample and diagnostic reference standard may be from bone marrow, or both samples may be from blood.

In one embodiment the diagnostic reference standard is a non-myeloid leukaemia reference standard, such as from a subject that does not have myeloid leukaemia (e.g. does not have CML or AML). Where the concentration of the cells are the same in the sample and diagnostic reference standard, this may indicate the absence of myeloid leukaemia.

Where the cell is a CMP cell an increased concentration of said cell in a sample when compared to said diagnostic reference standard may indicate the presence of myeloid leukaemia (preferably acute myeloid leukaemia, e.g. BP-CML). Likewise, no change in concentration of said cell in said sample when compared to the diagnostic reference standard may indicate the absence of myeloid leukaemia, or a decrease in concentration of said cell in said sample when compared to the diagnostic reference standard may indicate the presence of chronic phase chronic myeloid leukaemia (CP-CML). Where the cell is a MPP cell a decreased concentration of said cell in said sample when compared to the diagnostic reference standard may indicate the presence of myeloid leukaemia. Likewise, no change in concentration (or an increase in concentration) of said cell in said sample when compared to the diagnostic reference standard may indicate the absence of myeloid leukaemia. Where the cell is a LMPP cell an increased concentration of said cell in said sample when compared to the diagnostic reference standard may indicate the presence of myeloid leukaemia (preferably acute myeloid leukaemia, e.g. BP-CML). Likewise, no change in concentration (or a decrease in concentration) of said cell in said sample when compared to the diagnostic reference standard may indicate the absence of myeloid leukaemia or the presence of CP-CML or AP-CML. Where the cell is a GMP cell an increased concentration of said cell in said sample when compared to the diagnostic reference standard may indicate the presence of myeloid leukaemia (preferably acute myeloid leukaemia, e.g. BP-CML). Likewise, no change in concentration of said cell in said sample when compared to the diagnostic reference standard may indicate the absence of myeloid leukaemia, while a decrease in concentration of said cell may indicate the presence of CP-CML or AP-CML.

In one embodiment the diagnostic reference standard is a chronic phase chronic myeloid leukaemia (CP-CML) reference standard, e.g. is from a subject who has CP-CML. In another embodiment the diagnostic reference standard is an accelerated phase CML (AP-CML) (e.g. from a subject who has AP-CML). Where the concentration of the cells are the same in the sample and diagnostic reference standard, this may indicate the absence of myeloid leukaemia or the presence of CP-CML or AP-CML, respectively.

Where the cell is a CMP cell an increased concentration of said cell in said sample when compared to said diagnostic reference standard may indicate the presence of acute myeloid leukaemia (AML). Likewise, a decreased concentration or no change in concentration of said cell in said sample when compared to the diagnostic reference standard may indicate the absence AML. Where the cell is a MPP cell an increased concentration of said cell in said sample when compared to the diagnostic reference standard may indicate the presence of AML. Likewise, a decreased concentration or no change in concentration of said cell in said sample when compared to the diagnostic reference standard may indicate the absence of AML. Where the cell is a LMPP cell an increased concentration of said cell in said sample when compared to the diagnostic reference standard may indicate the presence of AML. Likewise, no change in concentration (or a decrease in concentration) of said cell in said sample when compared to the diagnostic reference standard may indicate the absence of AML (or the presence of CP-CML or AP-CML). Where the cell is a GMP cell an increased concentration of said cell in said sample when compared to the diagnostic reference standard may indicate the presence of AML. Likewise, no change in concentration of said cell in said sample when compared to the diagnostic reference standard may indicate the absence of myeloid leukaemia (or the presence of CP-CML or AP-CML).

Preferably the AML is BP-CML.

In one embodiment the diagnostic reference standard is an AML reference standard, such as from a subject with AML (e.g. BP-CML). In some embodiments where the concentration of the cells are the same in the sample and diagnostic reference standard, and/or in some embodiments wherein the concentration of cells in the sample is greater than in the diagnostic reference standard this may indicate the presence of AML.

Where the cell is a CMP cell an increased concentration or no change in concentration of said cell in said sample when compared to said diagnostic reference standard may indicate the presence of acute myeloid leukaemia (AML). Likewise, a decreased concentration of said cell in said sample when compared to the diagnostic reference standard may indicate the absence AML (or the presence of CP-CML or AP-CML). Where the cell is a MPP cell no change in concentration of said cell in said sample when compared to the diagnostic reference standard may indicate the presence of AML. Likewise, an increased or decreased concentration of said cell in said sample when compared to the diagnostic reference standard may indicate the absence of AML (or the presence of CP-CML or AP-CML). Where the cell is a LMPP cell an increased concentration or no change in concentration of said cell in said sample when compared to the diagnostic reference standard may indicate the presence of AML. Likewise, a decreased concentration of said cell in said sample when compared to the diagnostic reference standard may indicate the absence of AML (or the presence of CP-CML or AP-CML). Where the cell is a GMP cell an increased concentration or no change in concentration of said cell in said sample when compared to the diagnostic reference standard may indicate the presence of AML. Likewise, a decreased concentration of said cell in said sample when compared to the diagnostic reference standard may indicate the absence of myeloid leukaemia (or the presence of CP-CML or AP-CML).

Preferably the AML is BP-CML.

The increased or decreased concentration may be determined by any technique known to the skilled person. In one embodiment FACS is used to determine and quantify the concentration.

In one embodiment an increase in concentration is an increase of at least 0.5%, 1%, 2%, 3%, 4%, 5%, 6%, 7%, 8%, 9%, 10%, 11% or 12%. In one embodiment a decrease in concentration is a decrease of at least 0.5%, 1%, 2%, 3%, 4%, 5%, 6%, 7%, 8%, 9%, 10%, 11% or 12%.

The term “sample” as used herein refers to any sample containing a blood cell population. Suitably, the sample may be isolated from a subject suspected of having a myeloid leukaemia. In some embodiments the sample is isolated from a subject diagnosed as having a myeloid leukaemia.

The terms “subject” and “patient” are used synonymously herein. The “subject” may be a mammal, and preferably the subject is a human subject.

The sample may be a bone marrow or blood sample. The white blood cell population of the sample is preferably extracted or enriched prior to detection of the cell of the present invention. Methods suitable for extraction and/or enrichment of the white blood cells from a sample are conventional techniques known to those skilled in the art. By way of example, one approach is to deplete a sample of red blood cells by red cell lysis. Another approach is to isolate mononuclear cells by density centrifugation using a density media like Ficoll. CD34⁺ cells can be then be purified from mononuclear cells by incubation with magnetic beads coated with CD34 antibody and separating CD34⁺ cells using a magnet.

In one embodiment, the methods referred to herein are in vitro methods, such as ex vivo methods.

In one aspect the invention provides a method for identifying a therapeutic suitable for treating myeloid leukaemia, said method comprising:
a. contacting a sample with a therapeutic candidate, wherein said sample comprises LSCs;
b. incubating the sample and therapeutic candidate;
c. detecting the presence or absence of the LSCs; and
d. comparing the number of LSCs detected in c. with the number of LSCs detected in the isolated sample before step a.;
wherein the therapeutic candidate is identified as a therapeutic suitable for treating myeloid leukaemia when the relative number of LSCs is decreased after contact with the therapeutic candidate; or
wherein the therapeutic candidate is not identified as a therapeutic suitable for treating myeloid leukaemia when the relative number of LSCs is not decreased after contact with the therapeutic candidate; and
wherein the LSCs are detected by a method comprising:
   i. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample;
   ii. comparing the detected expression of said one or more genes to expression of said one or more genes in a reference standard; and
   iii. identifying the presence or absence of an LSC in said sample based on said comparison.

Preferably the method comprises detecting expression of one or more of: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4.

In related aspects, the invention provides a method for identifying a therapeutic suitable for treating myeloid leukaemia, said method comprising:
a. contacting a sample with a therapeutic candidate, wherein said sample comprises LSCs comprising a cell surface polypeptide marker phenotype:
i. CD34⁺; CD45RA⁻; CD123⁺; and CD38⁺; or
ii. CD34⁺, CD45RA⁻; CD90⁻; and CD38⁻;
wherein (+) indicates the presence and (−) indicates the absence of said cell surface polypeptide markers;
b. incubating the sample and therapeutic candidate;
c. detecting the presence or absence of the LSCs; and
d. comparing the number of LSCs detected in c. with the number of LSCs detected in the isolated sample before step a.;
wherein the therapeutic candidate is identified as a therapeutic suitable for treating myeloid leukaemia when the relative number of LSCs is decreased after contact with the therapeutic candidate; or
wherein the therapeutic candidate is not identified as a therapeutic suitable for treating myeloid leukaemia when the relative number of LSCs is not decreased after contact with the therapeutic candidate.

In one aspect the invention provides a method for monitoring efficacy of a therapeutic molecule in treating myeloid leukaemia, said method comprising:
a. providing an isolated sample from a patient administered the therapeutic molecule;
b. detecting the presence or absence of LSCs in said sample;
c. determining the relative number of said LSCs by comparing the number of LSCs detected in b. with the number of LSCs present in an isolated sample from the patient prior to administration of the therapeutic molecule;
d. confirming efficacy of the therapeutic molecule by identifying a relative decrease in the number of LSCs after contact with the therapeutic molecule; or confirming the absence of efficacy of the therapeutic molecule by identifying no decrease or an increase in the number of LSCs after contact with the therapeutic molecule; and wherein the LSCs are detected by a method comprising:
   i. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample;
   ii. comparing the detected expression of said one or more genes to expression of said one or more genes in a reference standard; and
   iii. identifying the presence or absence of an LSC in said sample based on said comparison.

Preferably the method comprises detecting the presence of one or more of: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4.

In a related aspect the invention provides a method for monitoring efficacy of a therapeutic molecule in treating myeloid leukaemia, said method comprising:
a. providing an isolated sample from a patient administered the therapeutic molecule;
b. detecting the presence or absence of LSCs in said sample, wherein said LSC comprises a cell surface polypeptide marker phenotype:
i. CD34⁺; CD45RA⁻; CD123⁺; and CD38⁺; or
ii. CD34⁺, CD45RA⁻; CD90⁻; and CD38⁻;
wherein (+) indicates the presence and (−) indicates the absence of said cell surface polypeptide markers;
c. determining the relative number of said LSCs by comparing the number of LSCs detected in b. with the number of LSCs present in an isolated sample from the
patient prior to administration of the therapeutic molecule;
d. confirming efficacy of the therapeutic molecule by identifying a relative decrease in the number of LSCs after administration with the therapeutic molecule; or
confirming the absence of efficacy of the therapeutic molecule by identifying no decrease or an increase in the number of LSCs after administration with the therapeutic molecule.

The methods described herein may also comprise a step of treating a myeloid leukaemia. In one aspect, the invention provides a method of treating myeloid leukaemia comprising:
a. obtaining the results of a method of the invention; and
b. treating myeloid leukaemia when myeloid leukaemia is diagnosed or when said LSC is present (preferably when said LSC is present).

Said method may preferably further comprise:
c. detecting whether the LSC is present after treatment (e.g. using a gene expression profile or method described herein); and
d. re-administering said treatment when said LSC is present.

In certain embodiments, the treatment may be administration of a medicament such as a therapeutic agent, e.g. a chemotherapeutic agent, allogeneic stem cell/bone marrow transplant or a treatment regimen such as radiotherapy. Typical chemotherapeutic agents may include anthracyclines (e.g. daunorubicin), purine analogues (e.g. fludarabine), cytarabine and epigenetic modifiers such as Azacitidine. Supportive therapies (e.g. to treat one or more symptoms of myeloid leukaemia) may also be offered in the form of blood product transfusion and antibiotic treatment of infections.

There is also provided a method comprising detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1.

Preferably the method comprises detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4.

In another aspect there is provided a method comprising detecting the presence or absence of a leukaemic stem cell (LSC) in a sample; wherein the LSC comprises a cell surface polypeptide marker phenotype:
a. CD34⁺; CD45RA⁻; CD123⁺; and CD38⁺; or
b. CD34⁺, CD45RA⁻; CD90⁻; and CD38⁻;
   wherein (+) indicates the presence and (−) indicates the absence of said cell surface polypeptide markers.

Embodiments described herein in respect of methods of the invention are intended to be applied to other methods of the invention, the uses, LSCs, kits, and compositions, and vice versa.

### Sequence Identity

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004). Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

ALIGNMENT SCORES FOR DETERMINING SEQUENCE IDENTITY
A R N D C Q E G H I L K M F P S T W Y V
A 4
R −1 5
N −2 0 6
D −2 −2 1 6
C 0 −3 −3 −3 9
Q −1 1 0 0 −3
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0−1 0 0 0 −1 −2 −2 0 −1 −2 −1 4
T 0 −1 0 −1 −1 −1 −1 −2 −2 −1 −1 −1 −1 −2 −1 1 5 −2
11
W −3 −3 −4 −4 −2 −2 −3 −2 −2 −3 −2 −3 −1 1 −4 −3
Y −2 −2 −2 −3 −2 −1 −2 −3 2 −1 −1 −2 −1 3 −3 −2 −2
2 7
V 0 −3 −3 −3 −1 −2 −2 −3 −3 3 1 −2 1 −1 −2 −2 0 −3
−1 4

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively. The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a LSC” includes a plurality of such candidate agents and reference to “the LSC” includes reference to one or more fatty acids and equivalents thereof known to those skilled in the art.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows expansion of LMPP- and GMP-like and MPP- and CMP-like populations in myeloid BP-CML. Representative FACS plots of CD34+ enriched (A) normal bone marrow; (B) CP-CML; (C) AP-CML; myeloid BP-CML with (D) MPP-like and CMP-like populations; or (E) LMPP-like and GMP-like populations. Numbers of samples studied is shown on the right. Markers studied are shown below plots. Numbers in gates are the mean of all samples within the group expressed as a % of Lin-CD34+ cells.

FIG. 2 shows dynamic changes in immunophenotypic compartments in the progression from CP to BP-CML. (A) Bar graphs of mean sizes of indicated populations (x-axis) as a percentage of bone marrow Lin-CD34+ population (y-axis). Error bar corresponds to standard error of the mean, *P<0.05: **P<0.01; **P<0.001. (B) Tabular representation of the data in (A).

FIG. 3 shows functional LSC activity in Myeloid BP-CML. (A) Purities of immunophenotypic populations (expressed as %), after FACS sorting, used in primary xenotransplantation from 5 patients (COL091, COL091R, CML371, CML002 and HER002). (B) Number of mice with human cell engraftment above engraftment threshold (defined as 0.1% human CD45+CD33+CD19− cells) out of the total number of mice injected. Myeloid engraftment or absence of engraftment is indicated. ND, non-detected. (C) Primary engraftment (4 patients—COL091, COL091R, CML371, and CML002—x-axis) in 1-6 mice from the indicated population. Black and white-centred dots show the engraftment level in individual mice. Y-axis: mean % human (h)CD45+CD33+CD19− cell engraftment/total live MNC. X-axis: injected cell fraction. Dashed horizontal line: engraftment threshold. (D) Number of human cells (Y-axis) injected in primary mice from indicated population from 4 patients, COL091, CML371, COL091R and CML002 (x-axis) in 1-6 mice. Black dots: engrafted mice. White-centred dots: mice that failed to engraft above the engraftment level of 0.1%.

FIG. 4 shows hierarchical relationships of normal HSPCs are not maintained in BPCML. (A) Percentage purities of population, after FACS sorting, injected for secondary transplant from 2 patients (COL091, CML002). (B) Secondary engraftment of cells from 2 patients (COL091 and CML002). X-axis: Bottom: patient sample populations injected into primary mice. Top: population from primary engrafted mouse injected into secondary recipient. Y-axis: mean % human (h)CD45+CD33+CD19− cell engraftment/total live MNC. Dashed horizontal line: engraftment threshold. Each dot represents one injected mouse: Black dots: engrafted mice. White-centred dots: mice that failed to engraft.

FIG. 5 shows clonal structures of stem/progenitor populations in Myeloid BP-CML. (A-C) Data from patients CML002, COL091 and COL091R. Clone identities denoted by circles or bars. i) Immunophenotypic HSPC populations in patient sample. Y-axis: Proportion of population as % of Lin-CD34+ cells. ii) Clonal composition of purified patient populations is based on FISH analysis. X-axis: HSPC population. Y-axis: Frequency of clones per population. iii) Clonal hierarchies inferred from FISH data (D) FISH images: ABL (red); BCR (green), BCR-ABL fusion (F), p53/17p13 (gold), MPO/17q22 (aqua). Atypical BCR-ABL is defined by 1 or 3 BCR-ABL fusions. 17p13 loss is defined by p53 loss. Isochrosome 17q is defined by 3 MPO signals. Numbers of signals detected is indicated for each sample.

FIG. 6 shows analysis of clonal structures in NSG mice after transplantation of stem/progenitor populations. Frequency and type of leukemic clones in individual engrafted mice. Injected populations indicated. Results from 1° (A, B, C) and 2° transplantation (A and B) are shown. NA: cells unavailable for FISH analysis.

FIG. 7 shows increased expression of MLNR, TIGIT, and CNGA1 for MPP cells compared to other HSCs and progenitor cells.

FIG. 8 shows increased expression of MME for LMPP cells compared to other HSCs and progenitor cells (except for MLP cells).

FIG. 9 shows increased expression of MS4A2 for CMP cells compared to other HSCs and progenitor cells.

FIG. 10 shows increased expression of SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and ILDR1 for GMP cells compared to other HSCs and progenitor cells.

FIG. 11 shows expression of levels of GYPE, MME, CCR7, CNTNAP2, ABCA13, SHH, ILDR1, LRP1, TMEM231, NFASC, MCOLN2, SLC24A2, SMIM24, NDUFB1, RAB11FIP3, SPN, JAM2, PIGO, SERPINE2, PILRB, ATG9B, SLC35F6, GPR63, GLG1, SLC35F5, PCDHB9, DERL1, FZD4, PKN2, CD46, CD55, LEPROT, CD83, HSPA5, TMCO3, TFRC, PDIA3, SLC9B1, TEX10, AQP11, SHISA9, CD99, SORCS1, IL1RAP, ITM2B, VAMP2, CNGB1, UBR4, RHOA, EMP3, CMTM6, SAMD8, IL6ST, SLC2A14, TAPT1, SLC12A8, CCDC47, LPAR4, CLIC4, SLC2A3, VAMP7, PTPRC, ITGAX, PIEZO2, IL18R1, PDGFA, IFITM1, ATP6AP2, RNF19B, CD36, LPAR6, TMEM50A, AIMP1, and SPCS1 (from top to bottom) for LSC LMPPs and GMPs compared to non-LSC LMPPs and GMPs.

SEQUENCE LISTING

| | Nucleotide/ | | |
|---|---|---|---|
| SEQ ID NO. | Polypeptide ID | Sequence Type | GenBank Accession Number |
| SEQ ID NO. 1 | MME | Genomic DNA | NC_000003.12 |
| SEQ ID NO. 2 | IFITM1 | Genomic DNA | NC_000011.10 |
| SEQ ID NO. 3 | CMTM6 | Genomic DNA | NC_000003.12 |
| SEQ ID NO. 4 | CD55 | Genomic DNA | NC_000001.11 |
| SEQ ID NO. 5 | SLC35F5 | Genomic DNA | NC_000002.12 |
| SEQ ID NO. 6 | CNTNAP2 | Genomic DNA | NC_000007.14 |
| SEQ ID NO. 7 | PIGO | Genomic DNA | NC_000009.12 |
| SEQ ID NO. 8 | SHH | Genomic DNA | NC_000007.14 |
| SEQ ID NO. 9 | AQP11 | Genomic DNA | NC_000011.10 |
| SEQ ID NO. 10 | PCDHB9 | Genomic DNA | NC_000005.10 |
| SEQ ID NO. 11 | RHOA | Genomic DNA | NC_000003.12 |
| SEQ ID NO. 12 | TMEM231 | Genomic DNA | NC_000016.10 |
| SEQ ID NO. 13 | SAMD8 | Genomic DNA | NC_000010.11 |
| SEQ ID NO. 14 | ABCA13 | Genomic DNA | NC_000007.14 |
| SEQ ID NO. 15 | TAPT1 | Genomic DNA | NC_000004.12 |
| SEQ ID NO. 16 | NFASC | Genomic DNA | NC_000001.11 |
| SEQ ID NO. 17 | LEPROT | Genomic DNA | NC_000001.11 |
| SEQ ID NO. 18 | MCOLN2 | Genomic DNA | NC_000001.11 |
| SEQ ID NO. 19 | IL6ST | Genomic DNA | NC_000005.10 |
| SEQ ID NO. 20 | EMP3 | Genomic DNA | NC_000019.10 |
| SEQ ID NO. 21 | CD83 | Genomic DNA | NC_000006.12 |
| SEQ ID NO. 22 | LPAR6 | Genomic DNA | NC_000013.11 |
| SEQ ID NO. 23 | PIEZO2 | Genomic DNA | NC_000018.10 |
| SEQ ID NO. 24 | DERL1 | Genomic DNA | NC_000008.11 |
| SEQ ID NO. 25 | IL1RAP | Genomic DNA | NC_000003.12 |
| SEQ ID NO. 26 | LPAR4 | Genomic DNA | NC_000023.11 |
| SEQ ID NO. 27 | SERPINE2 | Genomic DNA | NC_000002.12 |
| SEQ ID NO. 28 | PKN2 | Genomic DNA | NC_000001.11 |
| SEQ ID NO. 29 | VAMP2 | Genomic DNA | NC_000017.11 |
| SEQ ID NO. 30 | TMCO3 | Genomic DNA | NC_000013.11 |
| SEQ ID NO. 31 | VAMP7 | Genomic DNA | NC_000023.11 |
| SEQ ID NO. 32 | PTPRC | Genomic DNA | NC_000001.11 |
| SEQ ID NO. 33 | TRFC | Genomic DNA | NC_000003.12 |
| SEQ ID NO. 34 | ILDR1 | Genomic DNA | NC_000003.12 |
| SEQ ID NO. 35 | PDIA3 | Genomic DNA | NC_000015.10 |
| SEQ ID NO. 36 | AIMP1 | Genomic DNA | NC_000004.12 |
| SEQ ID NO. 37 | GPR63 | Genomic DNA | NC_000006.12 |
| SEQ ID NO. 38 | CCR7 | Genomic DNA | NC_000017.11 |
| SEQ ID NO. 39 | ATG9B | Genomic DNA | NC_000007.14 |
| SEQ ID NO. 40 | SLC9B1 | Genomic DNA | NC_000004.12 |
| SEQ ID NO. 41 | CD99 | Genomic DNA | NC_000023.11 |
| SEQ ID NO. 42 | LRP1 | Genomic DNA | NC_000012.12 |
| SEQ ID NO. 43 | UBR4 | Genomic DNA | NC_000001.11 |
| SEQ ID NO. 44 | ATP6AP2 | Genomic DNA | NC_000023.11 |
| SEQ ID NO. 45 | TEX10 | Genomic DNA | NC_000009.12 |
| SEQ ID NO. 46 | CNGB1 | Genomic DNA | NC_000016.10 |
| SEQ ID NO. 47 | SPN | Genomic DNA | NC_000016.10 |
| SEQ ID NO. 48 | PILRB | Genomic DNA | NC_000007.14 |
| SEQ ID NO. 49 | JAM2 | Genomic DNA | NC_000021.9 |
| SEQ ID NO. 50 | PDGFA | Genomic DNA | NC_000007.14 |
| SEQ ID NO. 51 | CD46 | Genomic DNA | NC_000001.11 |
| SEQ ID NO. 52 | NDUFB1 | Genomic DNA | NC_000014.9 |
| SEQ ID NO. 53 | GYPE | Genomic DNA | NC_000004.12 |
| SEQ ID NO. 54 | SLC12A8 | Genomic DNA | NC_000003.12 |
| SEQ ID NO. 55 | SLC2A14 | Genomic DNA | NC_000012.12 |
| SEQ ID NO. 56 | RNF19B | Genomic DNA | NC_000001.11 |
| SEQ ID NO. 57 | SPCS1 | Genomic DNA | NC_000003.12 |
| SEQ ID NO. 58 | SLC35F6 | Genomic DNA | NC_000002.12 |
| SEQ ID NO. 59 | CD36 | Genomic DNA | NC_000007.14 |
| SEQ ID NO. 60 | ITM2B | Genomic DNA | NC_000013.11 |
| SEQ ID NO. 61 | GLG1 | Genomic DNA | NC_000016.10 |
| SEQ ID NO. 62 | SMIM24 | Genomic DNA | NC_000019.10 |
| SEQ ID NO. 63 | TMEM50A | Genomic DNA | NC_000001.11 |
| SEQ ID NO. 64 | HSPA5 | Genomic DNA | NC_000009.12 |
| SEQ ID NO. 65 | ITGAX | Genomic DNA | NC_000016.10 |
| SEQ ID NO. 66 | SLC24A2 | Genomic DNA | NC_000009.12 |
| SEQ ID NO. 67 | SLC2A3 | Genomic DNA | NC_000012.12 |
| SEQ ID NO. 68 | RAB11FIP3 | Genomic DNA | NC_000016.10 |
| SEQ ID NO. 69 | IL18R1 | Genomic DNA | NC_000002.12 |
| SEQ ID NO. 70 | CCDC47 | Genomic DNA | NC_000017.11 |
| SEQ ID NO. 71 | FZD4 | Genomic DNA | NC_000011.10 |
| SEQ ID NO. 72 | SHISA9 | Genomic DNA | NC_000016.10 |
| SEQ ID NO. 73 | SORCS1 | Genomic DNA | NC_000010.11 |
| SEQ ID NO. 74 | CLIC4 | Genomic DNA | NC_000001.11 |
| SEQ ID NO. 75 | MS4A2 | Genomic DNA | NC_000011.10 |
| SEQ ID NO. 76 | MLNR | Genomic DNA | NC_000013.11 |
| SEQ ID NO. 77 | TIGIT | Genomic DNA | NC_000003.12 |
| SEQ ID NO. 78 | CNGA1 | Genomic DNA | NC_000004.12 |
| SEQ ID NO. 79 | SIRPB2 | Genomic DNA | NC_000020.11 |
| SEQ ID NO. 80 | PRRG4 | Genomic DNA | NC_000011.10 |
| SEQ ID NO. 81 | VSTM4 | Genomic DNA | NC_000010.11 |
| SEQ ID NO. 82 | TMEM107 | Genomic DNA | NC_000017.11 |
| SEQ ID NO. 83 | NETO2 | Genomic DNA | NC_000016.10 |
| SEQ ID NO. 84 | CSF1R | Genomic DNA | NC_000005.10 |
| SEQ ID NO. 85 | ADRB2 | Genomic DNA | NC_000005.10 |
| SEQ ID NO. 86 | TLR2 | Genomic DNA | NC_000004.12 |
| SEQ ID NO. 87 | FUT4 | Genomic DNA | NC_000011.10 |
| SEQ ID NO. 88 | MGST1 | Genomic DNA | NC_000012.12 |
| SEQ ID NO. 89 | CSF3R | Genomic DNA | NC_000001.11 |
| SEQ ID NO. 90 | HLA-B | Genomic DNA | NC_000006.12 |
| SEQ ID NO. 91 | ITGA10 | Genomic DNA | NC_000001.11 |
| SEQ ID NO. 92 | SLC26A8 | Genomic DNA | NC_000006.12 |
| SEQ ID NO. 93 | SIRPB1 | Genomic DNA | NC_000020.11 |
| SEQ ID NO. 94 | RAET1E | Genomic DNA | NC_000006.12 |
| SEQ ID NO. 95 | ST3GAL6 | Genomic DNA | NC_000003.12 |
| SEQ ID NO. 96 | LAMP1 | Genomic DNA | NC_000013.11 |
| SEQ ID NO. 97 | LGALS1 | Genomic DNA | NC_000022.11 |
| SEQ ID NO. 98 | MME | cDNA Transcript | NM_000902.3 |
| | | Variant 1 | |
| SEQ ID NO. 99 | MME | cDNA Transcript | NM_001354642.1 |
| | | Variant 3 | |
| SEQ ID NO. 100 | MME | cDNA Transcript | NM_001354643.1 |
| | | Variant 4 | |
| SEQ ID NO. 101 | MME | cDNA Transcript | NM_001354644.1 |
| | | Variant 5 | |
| SEQ ID NO. 102 | MME | cDNA Transcript | NM_007287.2 |
| | | Variant 1bis | |
| SEQ ID NO. 103 | MME | cDNA Transcript | NM_007288.3 |
| | | Variant 2a | |
| SEQ ID NO. 104 | MME | cDNA Transcript | NM_007289.3 |
| | | Variant 2b | |
| SEQ ID NO. 105 | MME | Amino Acid | NP_000893.2 |
| | | Sequence | |
| SEQ ID NO. 106 | MME | Amino Acid | NP_001341571.1 |
| | | Sequence | |
| SEQ ID NO. 107 | MME | Amino Acid | NP_001341572.1 |
| | | Sequence | |
| SEQ ID NO. 108 | MME | Amino Acid | NP_001341573.1 |
| | | Sequence | |
| SEQ ID NO. 109 | MME | Amino Acid | NP_009218.2 |
| | | Sequence | |
| SEQ ID NO. 110 | MME | Amino Acid | NP_009219.2 |
| | | Sequence | |
| SEQ ID NO. 111 | MME | Amino Acid | NP_009220.2 |
| | | Sequence | |
| SEQ ID NO. 112 | MME | mRNA Transcript | NM_007287.2 |
| | | Variant 1bis | |
| SEQ ID NO. 113 | MME | mRNA Transcript | NM_000902.3 |
| | | Variant 1 | |
| SEQ ID NO. 114 | MME | mRNA Transcript | XM_011512856.2 |
| | | Variant X2 | |
| SEQ ID NO. 115 | MME | mRNA Transcript | XM_011512857.2 |
| | | Variant X4 | |
| SEQ ID NO. 116 | MME | mRNA Transcript | XM_006713647.3 |
| | | Variant X5 | |
| SEQ ID NO. 117 | MME | mRNA Transcript | NM_007289.3 |
| | | Variant 2b | |
| SEQ ID NO. 118 | MME | mRNA Transcript | NM_007288.3 |
| | | Variant 2a | |
| SEQ ID NO. 119 | MME | mRNA Transcript | NM_001354643.1 |
| | | Variant 4 | |
| SEQ ID NO. 120 | MME | mRNA Transcript | NM_001354644.1 |
| | | Variant 5 | |
| SEQ ID NO. 121 | MME | mRNA Transcript | NM_001354642.1 |
| | | Variant 3 | |
| SEQ ID NO. 122 | IFITM1 | mRNA | NM_003641.3 |
| SEQ ID NO. 123 | CMTM6 | mRNA | NM_017801.2 |
| SEQ ID NO. 124 | CD55 | mRNA Transcript | NM_000574.4 |
| | | Variant 1 | |
| SEQ ID NO. 125 | CD55 | mRNA Transcript | NM_001114752.2 |
| | | Variant 2 | |
| SEQ ID NO. 126 | CD55 | mRNA Transcript | NM_001300902.1 |
| | | Variant 5 | |
| SEQ ID NO. 127 | CD55 | mRNA Transcript | NM_001300903.1 |
| | | Variant 6 | |
| SEQ ID NO. 128 | CD55 | mRNA Transcript | NM_001300904.1 |
| | | Variant 7 | |
| SEQ ID NO. 129 | CD55 | non-coding RNA | NR_125349.1 |
| | | Transcript Variant 8 | |
| SEQ ID NO. 130 | CD55 | mRNA Transcript | XM_017000467.1 |
| | | Variant X1 | |
| SEQ ID NO. 131 | SLC35F5 | mRNA Transcript | XM_017005027.1 |
| | | Variant X2 | |
| SEQ ID NO. 132 | SLC35F5 | mRNA Transcript | XM_011511922.2 |
| | | Variant X3 | |
| SEQ ID NO. 133 | SLC35F5 | mRNA Transcript | XM_011511923.2 |
| | | Variant X4 | |
| SEQ ID NO. 134 | SLC35F5 | mRNA Transcript | NM_001330314.1 |
| | | Variant 3 | |
| SEQ ID NO. 135 | SLC35F5 | mRNA Transcript | NM_025181.4 |
| | | Variant 1 | |
| SEQ ID NO. 136 | SLC35F5 | mRNA Transcript | NM_001330317.1 |
| | | Variant 6 | |
| SEQ ID NO. 137 | SLC35F5 | mRNA Transcript | NM_001330316.1 |
| | | Variant 5 | |
| SEQ ID NO. 138 | SLC35F5 | mRNA Transcript | NM_001330315.1 |
| | | Variant 4 | |
| SEQ ID NO. 139 | SLC35F5 | non-coding RNA | NR_104470.2 |
| | | Transcript Variant 2 | |
| SEQ ID NO. 140 | CNTNAP2 | mRNA | NM_014141.5 |
| SEQ ID NO. 141 | CNTNAP2 | mRNA Transcript | XM_017011950.1 |
| | | Variant X1 | |
| SEQ ID NO. 142 | PIGO | mRNA Transcript | NM_032634.3 |
| | | Variant 1 | |
| SEQ ID NO. 143 | PIGO | mRNA Transcript | NM_152850.3 |
| | | Variant 2 | |
| SEQ ID NO. 144 | PIGO | mRNA Transcript | NM_001201484.1 |
| | | Variant 3 | |
| SEQ ID NO. 145 | PIGO | mRNA Transcript | XM_005251619.3 |
| | | Variant X1 | |
| SEQ ID NO. 146 | PIGO | mRNA Transcript | XM_017015222.1 |
| | | Variant X2 | |
| SEQ ID NO. 147 | PIGO | misc RNA Transcript | XR_242515.2 |
| | | Variant X3 | |
| SEQ ID NO. 148 | PIGO | misc RNA Transcript | XR_001746390.1 |
| | | Variant X4 | |
| SEQ ID NO. 149 | PIGO | mRNA Transcript | XM_017015223.1 |
| | | Variant X5 | |
| SEQ ID NO. 150 | PIGO | mRNA Transcript | XM_017015224.1 |
| | | Variant X6 | |
| SEQ ID NO. 151 | PIGO | misc RNA Transcript | XR_001746391.1 |
| | | Variant X7 | |
| SEQ ID NO. 152 | SHH | mRNA Transcript | NM_000193.3 |
| | | Variant 1 | |
| SEQ ID NO. 153 | SHH | mRNA Transcript | NM_001310462.1 |
| | | Variant 2 | |
| SEQ ID NO. 154 | SHH | non-coding RNA | NR_132318.1 |
| | | Transcript Variant 3 | |
| SEQ ID NO. 155 | SHH | non-coding RNA | NR_132319.1 |
| | | Transcript Variant 4 | |
| SEQ ID NO. 156 | SHH | mRNA Transcript | XM_011516479.2 |
| | | Variant X1 | |
| SEQ ID NO. 157 | SHH | mRNA Transcript | XM_011516480.2 |
| | | Variant X2 | |
| SEQ ID NO. 158 | AQP11 | mRNA | NM_173039.2 |
| SEQ ID NO. 159 | AQP11 | mRNA Transcript | XM_005273917.4 |
| | | Variant X1 | |
| SEQ ID NO. 160 | PCDHB9 | mRNA | NM_019119.4 |
| SEQ ID NO. 161 | RHOA | mRNA Transcript | NM_001664.3 |
| | | Variant 1 | |
| SEQ ID NO. 162 | RHOA | mRNA Transcript | NM_001313941.1 |
| | | Variant 2 | |
| SEQ ID NO. 163 | RHOA | mRNA Transcript | NM_001313943.1 |
| | | Variant 3 | |
| SEQ ID NO. 164 | RHOA | mRNA Transcript | NM_001313944.1 |
| | | Variant 4 | |
| SEQ ID NO. 165 | RHOA | mRNA Transcript | NM_001313945.1 |
| | | Variant 5 | |
| SEQ ID NO. 166 | RHOA | mRNA Transcript | NM_001313946.1 |
| | | Variant 6 | |
| SEQ ID NO. 167 | RHOA | mRNA Transcript | NM_001313947.1 |
| | | Variant 7 | |
| SEQ ID NO. 168 | TMEM231 | mRNA Transcript | NM_001077416.2 |
| | | Variant 1 | |
| SEQ ID NO. 169 | TMEM231 | non-coding RNA | NR_074083.1 |
| | | Transcript Variant 5 | |
| SEQ ID NO. 170 | TMEM231 | mRNA Transcript | NM_001077418.2 |
| | | Variant 2 | |
| SEQ ID NO. 171 | SAMD8 | mRNA Transcript | NM_144660.2 |
| | | Variant 2 | |
| SEQ ID NO. 172 | SAMD8 | mRNA Transcript | NM_001174156.1 |
| | | Variant 1 | |
| SEQ ID NO. 173 | SAMD8 | mRNA Transcript | XM_011539311.1 |
| | | Variant X1 | |
| SEQ ID NO. 174 | SAMD8 | mRNA Transcript | XM_017015738.1 |
| | | Variant X2 | |
| SEQ ID NO. 175 | SAMD8 | mRNA Transcript | XM_005269541.4 |
| | | Variant X3 | |
| SEQ ID NO. 176 | SAMD8 | mRNA Transcript | XM_017015739.1 |
| | | Variant X4 | |
| SEQ ID NO. 177 | SAMD8 | mRNA Transcript | XM_011539312.2 |
| | | Variant X5 | |
| SEQ ID NO. 178 | SAMD8 | misc RNA Transcript | XR_001747026.1 |
| | | Variant X6 | |
| SEQ ID NO. 179 | ABCA13 | mRNA | NM_152701.4 |
| SEQ ID NO. 180 | ABCA13 | mRNA Transcript | XM_011515130.2 |
| | | Variant X1 | |
| SEQ ID NO. 181 | ABCA13 | mRNA Transcript | XM_011515131.2 |
| | | Variant X2 | |
| SEQ ID NO. 182 | ABCA13 | mRNA Transcript | XM_011515132.2 |
| | | Variant X3 | |
| SEQ ID NO. 183 | ABCA13 | mRNA Transcript | XM_011515133.2 |
| | | Variant X4 | |
| SEQ ID NO. 184 | ABCA13 | misc RNA Transcript | XR_926914.2 |
| | | Variant X5 | |
| SEQ ID NO. 185 | ABCA13 | misc RNA Transcript | XR_001744555.1 |
| | | Variant X6 | |
| SEQ ID NO. 186 | ABCA13 | misc RNA Transcript | XR_926915.2 |
| | | Variant X7 | |
| SEQ ID NO. 187 | ABCA13 | mRNA Transcript | XM_011515134.2 |
| | | Variant X8 | |
| SEQ ID NO. 188 | ABCA13 | mRNA Transcript | XM_011515136.2 |
| | | Variant X9 | |
| SEQ ID NO. 189 | ABCA13 | misc RNA Transcript | XR_926916.2 |
| | | Variant X10 | |
| SEQ ID NO. 190 | ABCA13 | mRNA Transcript | XM_011515137.2 |
| | | Variant X11 | |
| SEQ ID NO. 191 | ABCA13 | misc RNA Transcript | XR_926919.2 |
| | | Variant X12 | |
| SEQ ID NO. 192 | ABCA13 | mRNA Transcript | XM_011515138.2 |
| | | Variant X13 | |
| SEQ ID NO. 193 | ABCA13 | mRNA Transcript | XM_011515139.2 |
| | | Variant X14 | |
| SEQ ID NO. 194 | ABCA13 | mRNA Transcript | XM_017011767.1 |
| | | Variant X15 | |
| SEQ ID NO. 195 | ABCA13 | mRNA Transcript | XM_017011768.1 |
| | | Variant X16 | |
| SEQ ID NO. 196 | ABCA13 | mRNA Transcript | XM_011515141.2 |
| | | Variant X17 | |
| SEQ ID NO. 197 | ABCA13 | mRNA Transcript | XM_011515142.2 |
| | | Variant X18 | |
| SEQ ID NO. 198 | ABCA13 | mRNA Transcript | XM_011515143.2 |
| | | Variant X19 | |
| SEQ ID NO. 199 | ABCA13 | mRNA Transcript | XM_011515144.2 |
| | | Variant X20 | |
| SEQ ID NO. 200 | ABCA13 | mRNA Transcript | XM_011515145.2 |
| | | Variant X21 | |
| SEQ ID NO. 201 | ABCA13 | mRNA Transcript | XM_011515146.2 |
| | | Variant X22 | |
| SEQ ID NO. 202 | ABCA13 | mRNA Transcript | XM_011515147.2 |
| | | Variant X23 | |
| SEQ ID NO. 203 | ABCA13 | mRNA Transcript | XM_011515148.2 |
| | | Variant X24 | |
| SEQ ID NO. 204 | ABCA13 | mRNA Transcript | XM_011515149.2 |
| | | Variant X25 | |
| SEQ ID NO. 205 | ABCA13 | mRNA Transcript | XM_017011769.1 |
| | | Variant X26 | |
| SEQ ID NO. 206 | TAPT1 | mRNA | NM_153365.2 |
| SEQ ID NO. 207 | TAPT1 | mRNA Transcript | XM_011513812.2 |
| | | Variant X1 | |
| SEQ ID NO. 208 | TAPT1 | mRNA Transcript | XM_005248139.2 |
| | | Variant X2 | |
| SEQ ID NO. 209 | TAPT1 | mRNA Transcript | XM_005248140.3 |
| | | Variant X3 | |
| SEQ ID NO. 210 | TAPT1 | mRNA Transcript | XM_011513815.2 |
| | | Variant X4 | |
| SEQ ID NO. 211 | TAPT1 | mRNA Transcript | XM_017007876.1 |
| | | Variant X5 | |
| SEQ ID NO. 212 | TAPT1 | misc RNA Transcript | XR_241676.3 |
| | | Variant X6 | |
| SEQ ID NO. 213 | TAPT1 | misc RNA Transcript | XR_925318.2 |
| | | Variant X7 | |
| SEQ ID NO. 214 | TAPT1 | mRNA Transcript | XM_011513816.2 |
| | | Variant X8 | |
| SEQ ID NO. 215 | TAPT1 | mRNA Transcript | XM_011513817.2 |
| | | Variant X9 | |
| SEQ ID NO. 216 | NFASC | mRNA Transcript | NM_001005389.1 |
| | | Variant 5 | |
| SEQ ID NO. 217 | NFASC | mRNA Transcript | NM_015090.3 |
| | | Variant 4 | |
| SEQ ID NO. 218 | NFASC | mRNA Transcript | NM_001005388.2 |
| | | Variant 1 | |
| SEQ ID NO. 219 | NFASC | mRNA Transcript | NM_001160331.1 |
| | | Variant 2 | |
| SEQ ID NO. 220 | NFASC | mRNA Transcript | NM_001160332.1 |
| | | Variant 3 | |
| SEQ ID NO. 221 | NFASC | mRNA Transcript | NM_001160333.1 |
| | | Variant 6 | |
| SEQ ID NO. 222 | NFASC | mRNA Transcript | XM_011509311.1 |
| | | Variant X1 | |
| SEQ ID NO. 223 | NFASC | mRNA Transcript | XM_011509312.1 |
| | | Variant X2 | |
| SEQ ID NO. 224 | NFASC | mRNA Transcript | XM_011509313.1 |
| | | Variant X3 | |
| SEQ ID NO. 225 | NFASC | mRNA Transcript | XM_011509314.1 |
| | | Variant X5 | |
| SEQ ID NO. 226 | NFASC | mRNA Transcript | XM_011509315.1 |
| | | Variant X6 | |
| SEQ ID NO. 227 | NFASC | mRNA Transcript | XM_011509316.1 |
| | | Variant X8 | |
| SEQ ID NO. 228 | NFASC | mRNA Transcript | XM_011509317.1 |
| | | Variant X9 | |
| SEQ ID NO. 229 | NFASC | mRNA Transcript | XM_011509319.1 |
| | | Variant X11 | |
| SEQ ID NO. 230 | NFASC | mRNA Transcript | XM_011509321.1 |
| | | Variant X13 | |
| SEQ ID NO. 231 | NFASC | mRNA Transcript | XM_011509322.1 |
| | | Variant X15 | |
| SEQ ID NO. 232 | NFASC | mRNA Transcript | XM_011509323.1 |
| | | Variant X16 | |
| SEQ ID NO. 233 | NFASC | mRNA Transcript | XM_011509324.1 |
| | | Variant X17 | |
| SEQ ID NO. 234 | NFASC | mRNA Transcript | XM_011509325.1 |
| | | Variant X18 | |
| SEQ ID NO. 235 | NFASC | mRNA Transcript | XM_005244989.3 |
| | | Variant X19 | |
| SEQ ID NO. 236 | NFASC | mRNA Transcript | XM_005244991.3 |
| | | Variant X21 | |
| SEQ ID NO. 237 | NFASC | mRNA Transcript | XM_005244993.3 |
| | | Variant X23 | |
| SEQ ID NO. 238 | NFASC | mRNA Transcript | XM_011509326.1 |
| | | Variant X28 | |
| SEQ ID NO. 239 | NFASC | mRNA Transcript | XM_011509327.1 |
| | | Variant X29 | |
| SEQ ID NO. 240 | NFASC | mRNA Transcript | XM_005244997.3 |
| | | Variant X33 | |
| SEQ ID NO. 241 | NFASC | mRNA Transcript | XM_011509328.1 |
| | | Variant X35 | |
| SEQ ID NO. 242 | NFASC | mRNA Transcript | XM_017000733.1 |
| | | Variant X4 | |
| SEQ ID NO. 243 | NFASC | mRNA Transcript | XM_017000734.1 |
| | | Variant X7 | |
| SEQ ID NO. 244 | NFASC | mRNA Transcript | XM_011509318.2 |
| | | Variant X10 | |
| SEQ ID NO. 245 | NFASC | mRNA Transcript | XM_011509320.2 |
| | | Variant X12 | |
| SEQ ID NO. 246 | NFASC | mRNA Transcript | XM_017000735.1 |
| | | Variant X14 | |
| SEQ ID NO. 247 | NFASC | mRNA Transcript | XM_017000736.1 |
| | | Variant X20 | |
| SEQ ID NO. 248 | NFASC | mRNA Transcript | XM_005244992.4 |
| | | Variant X22 | |
| SEQ ID NO. 249 | NFASC | mRNA Transcript | XM_017000737.1 |
| | | Variant X24 | |
| SEQ ID NO. 250 | NFASC | misc RNA Transcript | XR_921759.2 |
| | | Variant X25 | |
| SEQ ID NO. 251 | NFASC | mRNA Transcript | XM_017000738.1 |
| | | Variant X26 | |
| SEQ ID NO. 252 | NFASC | mRNA Transcript | XM_017000739.1 |
| | | Variant X27 | |
| SEQ ID NO. 253 | NFASC | mRNA Transcript | XM_017000740.1 |
| | | Variant X30 | |
| SEQ ID NO. 254 | NFASC | mRNA Transcript | XM_017000741.1 |
| | | Variant X31 | |
| SEQ ID NO. 255 | NFASC | mRNA Transcript | XM_017000742.1 |
| | | Variant X32 | |
| SEQ ID NO. 256 | NFASC | mRNA Transcript | XM_017000743.1 |
| | | Variant X34 | |
| SEQ ID NO. 257 | LEPROT | mRNA Transcript | NM_001198681.1 |
| | | Variant 2 | |
| SEQ ID NO. 258 | LEPROT | mRNA Transcript | NM_017526.4 |
| | | Variant 1 | |
| SEQ ID NO. 259 | LEPROT | mRNA Transcript | NM_001198683.1 |
| | | Variant 3 | |
| SEQ ID NO. 260 | MCOLN2 | mRNA Transcript | NM_153259.3 |
| | | Variant 1 | |
| SEQ ID NO. 261 | MCOLN2 | mRNA Transcript | XM_011541187.2 |
| | | Variant X2 | |
| SEQ ID NO. 262 | MCOLN2 | mRNA Transcript | XM_011541188.2 |
| | | Variant X3 | |
| SEQ ID NO. 263 | MCOLN2 | misc RNA Transcript | XR_001737090.1 |
| | | Variant X4 | |
| SEQ ID NO. 264 | MCOLN2 | mRNA Transcript | XM_005270719.3 |
| | | Variant X5 | |
| SEQ ID NO. 265 | MCOLN2 | misc RNA Transcript | XR_001737091.1 |
| | | Variant X6 | |
| SEQ ID NO. 266 | MCOLN2 | mRNA Transcript | XM_017000923.1 |
| | | Variant X7 | |
| SEQ ID NO. 267 | MCOLN2 | mRNA Transcript | NM_001330647.1 |
| | | Variant 2 | |
| SEQ ID NO. 268 | IL6ST | mRNA Transcript | NM_002184.3 |
| | | Variant 1 | |
| SEQ ID NO. 269 | IL6ST | mRNA Transcript | NM_175767.2 |
| | | Variant 2 | |
| SEQ ID NO. 270 | IL6ST | mRNA Transcript | NM_001190981.1 |
| | | Variant 3 | |
| SEQ ID NO. 271 | IL6ST | non-coding RNA | NR_120480.1 |
| | | Transcript Variant 4 | |
| SEQ ID NO. 272 | IL6ST | misc RNA Transcript | XR_001742062.1 |
| | | Variant X1 | |
| SEQ ID NO. 273 | IL6ST | mRNA Transcript | XM_017009441.1 |
| | | Variant X2 | |
| SEQ ID NO. 274 | IL6ST | mRNA Transcript | XM_017009442.1 |
| | | Variant X3 | |
| SEQ ID NO. 275 | EMP3 | mRNA Transcript | NM_001425.2 |
| | | Variant 1 | |
| SEQ ID NO. 276 | EMP3 | mRNA Transcript | NM_001313905.1 |
| | | Variant 2 | |
| SEQ ID NO. 277 | EMP3 | mRNA Transcript | XM_011526605.2 |
| | | Variant X1 | |
| SEQ ID NO. 278 | CD83 | mRNA Transcript | NM_004233.3 |
| | | Variant 1 | |
| SEQ ID NO. 279 | CD83 | mRNA Transcript | NM_001040280.1 |
| | | Variant 2 | |
| SEQ ID NO. 280 | CD83 | mRNA Transcript | NM_001251901.1 |
| | | Variant 3 | |
| SEQ ID NO. 281 | LPAR6 | mRNA Transcript | NM_005767.5 |
| | | Variant 1 | |
| SEQ ID NO. 282 | LPAR6 | mRNA Transcript | NM_001162497.1 |
| | | Variant 2 | |
| SEQ ID NO. 283 | LPAR6 | mRNA Transcript | NM_001162498.1 |
| | | Variant 3 | |
| SEQ ID NO. 284 | PIEZO2 | mRNA | NM_022068.3 |
| SEQ ID NO. 285 | PIEZO2 | mRNA Transcript | XM_011525723.2 |
| | | Variant X1 | |
| SEQ ID NO. 286 | PIEZO2 | mRNA Transcript | XM_017025918.1 |
| | | Variant X2 | |
| SEQ ID NO. 287 | PIEZO2 | mRNA Transcript | XM_011525724.2 |
| | | Variant X3 | |
| SEQ ID NO. 288 | PIEZO2 | mRNA Transcript | XM_011525725.2 |
| | | Variant X4 | |
| SEQ ID NO. 289 | PIEZO2 | mRNA Transcript | XM_011525726.2 |
| | | Variant X5 | |
| SEQ ID NO. 290 | PIEZO2 | misc RNA Transcript | XR_001753259.1 |
| | | Variant X6 | |
| SEQ ID NO. 291 | DERL1 | mRNA Transcript | NM_024295.5 |
| | | Variant 1 | |
| SEQ ID NO. 292 | DERL1 | mRNA Transcript | NM_001134671.2 |
| | | Variant 2 | |
| SEQ ID NO. 293 | DERL1 | mRNA Transcript | XM_006716657.1 |
| | | Variant X2 | |
| SEQ ID NO. 294 | DERL1 | mRNA Transcript | NM_001330601.1 |
| | | Variant 3 | |
| SEQ ID NO. 295 | IL1RAP | mRNA Transcript | NM_002182.3 |
| | | Variant 1 | |
| SEQ ID NO. 296 | IL1RAP | mRNA Transcript | NM_134470.3 |
| | | Variant 2 | |
| SEQ ID NO. 297 | IL1RAP | mRNA Transcript | NM_001167928.1 |
| | | Variant 3 | |
| SEQ ID NO. 298 | IL1RAP | mRNA Transcript | NM_001167929.1 |
| | | Variant 4 | |
| SEQ ID NO. 299 | IL1RAP | mRNA Transcript | NM_001167930.1 |
| | | Variant 5 | |
| SEQ ID NO. 300 | IL1RAP | mRNA Transcript | NM_001167931.1 |
| | | Variant 6 | |
| SEQ ID NO. 301 | IL1RAP | mRNA Transcript | XM_017006347.1 |
| | | Variant X1 | |
| SEQ ID NO. 302 | IL1RAP | mRNA Transcript | XM_017006348.1 |
| | | Variant X2 | |
| SEQ ID NO. 303 | LPAR4 | mRNA Transcript | NM_005296.2 |
| | | Variant 2 | |
| SEQ ID NO. 304 | LPAR4 | mRNA Transcript | NM_001278000.1 |
| | | Variant 1 | |
| SEQ ID NO. 305 | LPAR4 | mRNA Transcript | XM_017029437.1 |
| | | Variant X1 | |
| SEQ ID NO. 306 | LPAR4 | mRNA Transcript | XM_005262126.3 |
| | | Variant X2 | |
| SEQ ID NO. 307 | LPAR4 | mRNA Transcript | XM_006724639.3 |
| | | Variant X3 | |
| SEQ ID NO. 308 | LPAR4 | mRNA Transcript | XM_017029438.1 |
| | | Variant X4 | |
| SEQ ID NO. 309 | SERPINE2 | mRNA Transcript | NM_006216.3 |
| | | Variant 1 | |
| SEQ ID NO. 310 | SERPINE2 | mRNA Transcript | NM_001136528.1 |
| | | Variant 2 | |
| SEQ ID NO. 311 | SERPINE2 | mRNA Transcript | NM_001136530.1 |
| | | Variant 4 | |
| SEQ ID NO. 312 | SERPINE2 | non-coding RNA | NR_073116.1 |
| | | Transcript Variant 3 | |
| SEQ ID NO. 313 | SERPINE2 | mRNA Transcript | XM_005246641.2 |
| | | Variant X1 | |
| SEQ ID NO. 314 | SERPINE2 | mRNA Transcript | XM_017004329.1 |
| | | Variant X2 | |
| SEQ ID NO. 315 | SERPINE2 | mRNA Transcript | XM_017004330.1 |
| | | Variant X3 | |
| SEQ ID NO. 316 | SERPINE2 | mRNA Transcript | XM_017004331.1 |
| | | Variant X4 | |
| SEQ ID NO. 317 | SERPINE2 | mRNA Transcript | XM_017004332.1 |
| | | Variant X5 | |
| SEQ ID NO. 318 | PKN2 | mRNA Transcript | NM_006256.3 |
| | | Variant 1 | |
| SEQ ID NO. 319 | PKN2 | mRNA Transcript | NM_001320709.1 |
| | | Variant 4 | |
| SEQ ID NO. 320 | PKN2 | mRNA Transcript | NM_001320707.1 |
| | | Variant 2 | |
| SEQ ID NO. 321 | PKN2 | mRNA Transcript | NM_001320708.1 |
| | | Variant 3 | |
| SEQ ID NO. 322 | PKN2 | mRNA Transcript | XM_017001782.1 |
| | | Variant X1 | |
| SEQ ID NO. 323 | PKN2 | mRNA Transcript | XM_017001783.1 |
| | | Variant X2 | |
| SEQ ID NO. 324 | PKN2 | mRNA Transcript | XM_011541772.2 |
| | | Variant X3 | |
| SEQ ID NO. 325 | VAMP2 | mRNA Transcript | NM_014232.2 |
| | | Variant 1 | |
| SEQ ID NO. 326 | VAMP2 | mRNA Transcript | NM_001330125.1 |
| | | Variant 2 | |
| SEQ ID NO. 327 | TMCO3 | mRNA Transcript | XM_006719969.1 |
| | | Variant X2 | |
| SEQ ID NO. 328 | TMCO3 | mRNA Transcript | XM_011537498.2 |
| | | Variant X1 | |
| SEQ ID NO. 329 | TMCO3 | mRNA Transcript | XM_017020630.1 |
| | | Variant X4 | |
| SEQ ID NO. 330 | TMCO3 | mRNA Transcript | XM_011537501.2 |
| | | Variant X8 | |
| SEQ ID NO. 331 | TMCO3 | mRNA Transcript | XM_011537502.2 |
| | | Variant X9 | |
| SEQ ID NO. 332 | TMCO3 | misc RNA Transcript | XR_001749587.1 |
| | | Variant X10 | |
| SEQ ID NO. 333 | TMCO3 | mRNA Transcript | XM_017020634.1 |
| | | Variant X11 | |
| SEQ ID NO. 334 | TMCO3 | mRNA Transcript | XM_011537504.2 |
| | | Variant X15 | |
| SEQ ID NO. 335 | TMCO3 | mRNA Transcript | XM_017020636.1 |
| | | Variant X16 | |
| SEQ ID NO. 336 | TMCO3 | misc RNA Transcript | XR_001749590.1 |
| | | Variant X17 | |
| SEQ ID NO. 337 | TMCO3 | mRNA Transcript | XM_011537506.2 |
| | | Variant X18 | |
| SEQ ID NO. 338 | TMCO3 | mRNA Transcript | XM_011537507.2 |
| | | Variant X19 | |
| SEQ ID NO. 339 | TMCO3 | misc RNA Transcript | XR_944279.2 |
| | | Variant X20 | |
| SEQ ID NO. 340 | TMCO3 | misc RNA Transcript | XR_001749591.1 |
| | | Variant X21 | |
| SEQ ID NO. 341 | TMCO3 | mRNA Transcript | XM_011537509.2 |
| | | Variant X22 | |
| SEQ ID NO. 342 | TMCO3 | mRNA Transcript | NM_001349745.1 |
| | | Variant 6 | |
| SEQ ID NO. 343 | TMCO3 | mRNA Transcript | NM_001349743.1 |
| | | Variant 4 | |
| SEQ ID NO. 344 | TMCO3 | mRNA Transcript | NM_001349741.1 |
| | | Variant 2 | |
| SEQ ID NO. 345 | TMCO3 | mRNA Transcript | NM_001349746.1 |
| | | Variant 7 | |
| SEQ ID NO. 346 | TMCO3 | mRNA Transcript | NM_001349744.1 |
| | | Variant 5 | |
| SEQ ID NO. 347 | TMCO3 | mRNA Transcript | NM_001349742.1 |
| | | Variant 3 | |
| SEQ ID NO. 348 | TMCO3 | mRNA Transcript | NM_017905.5 |
| | | Variant 1 | |
| SEQ ID NO. 349 | TMCO3 | non-coding RNA | NR_146221.1 |
| | | Transcript Variant 8 | |
| SEQ ID NO. 350 | TMCO3 | non-coding RNA | NR_146222.1 |
| | | Transcript Variant 9 | |
| SEQ ID NO. 351 | VAMP7 | mRNA Transcript | NM_001145149.2 |
| | | Variant 2 | |
| SEQ ID NO. 352 | VAMP7 | mRNA Transcript | NM_005638.5 |
| | | Variant 1 | |
| SEQ ID NO. 353 | VAMP7 | mRNA Transcript | NM_001185183.1 |
| | | Variant 3 | |
| SEQ ID NO. 354 | VAMP7 | non-coding RNA | NR_033714.1 |
| | | Transcript Variant 4 | |
| SEQ ID NO. 355 | VAMP7 | non-coding RNA | NR_033715.1 |
| | | Transcript Variant 5 | |
| SEQ ID NO. 356 | VAMP7 | mRNA Transcript | XM_011531188.1 |
| | | Variant X1 | |
| SEQ ID NO. 357 | V.AMP7 | mRNA Transcript | XM_011545653.1 |
| | | Variant X1 | |
| SEQ ID NO. 358 | V.AMP7 | mRNA Transcript | XM_017029760.1 |
| | | Variant X2 | |
| SEQ ID NO. 359 | V.AMP7 | mRNA Transcript | XM_017030063.1 |
| | | Variant X2 | |
| SEQ ID NO. 360 | PTPRC | mRNA Transcript | NM_001267798.1 |
| | | Variant 5 | |
| SEQ ID NO. 361 | PTPRC | mRNA Transcript | NM_002838.4 |
| | | Variant 1 | |
| SEQ ID NO. 362 | PTPRC | mRNA Transcript | NM_080921.3 |
| | | Variant 2 | |
| SEQ ID NO. 363 | PTPRC | non-coding RNA | NR_052021.1 |
| | | Transcript Variant 4 | |
| SEQ ID NO. 364 | PTPRC | mRNA Transcript | XM_006711472.3 |
| | | Variant X1 | |
| SEQ ID NO. 365 | PTPRC | mRNA Transcript | XM_006711473.3 |
| | | Variant X2 | |
| SEQ ID NO. 366 | PTPRC | mRNA Transcript | XM_006711474.3 |
| | | Variant X3 | |
| SEQ ID NO. 367 | TFRC | mRNA Transcript | NM_001128148.2 |
| | | Variant 2 | |
| SEQ ID NO. 368 | TFRC | mRNA Transcript | NM_003234.3 |
| | | Variant 1 | |
| SEQ ID NO. 369 | TFRC | mRNA Transcript | NM_001313965.1 |
| | | Variant 3 | |
| SEQ ID NO. 370 | TFRC | mRNA Transcript | NM_001313966.1 |
| | | Variant 4 | |
| SEQ ID NO. 371 | ILDR1 | mRNA Transcript | NM_175924.3 |
| | | Variant 2 | |
| SEQ ID NO. 372 | ILDR1 | mRNA Transcript | NM_001199799.1 |
| | | Variant 1 | |
| SEQ ID NO. 373 | ILDR1 | mRNA Transcript | NM_001199800.1 |
| | | Variant 3 | |
| SEQ ID NO. 374 | ILDR1 | mRNA Transcript | XM_011512738.2 |
| | | Variant X1 | |
| SEQ ID NO. 375 | ILDR1 | mRNA Transcript | XM_005247389.4 |
| | | Variant X2 | |
| SEQ ID NO. 376 | ILDR1 | mRNA Transcript | XM_011512739.2 |
| | | Variant X3 | |
| SEQ ID NO. 377 | ILDR1 | cDNA Transcript | NM_001199799.1 |
| | | Variant 1 | |
| SEQ ID NO. 378 | ILDR1 | Amino Acid | NP_001186728.1 |
| | | Sequence Isoform 1 | |
| | | Precursor | |
| SEQ ID NO. 379 | ILDR1 | cDNA Transcript | NM_001199800.1 |
| | | Variant 3 | |
| SEQ ID NO. 380 | ILDR1 | Amino Acid | NP_001186729.1 |
| | | Sequence Isoform 3 | |
| | | Precursor | |
| SEQ ID NO. 381 | ILDR1 | cDNA Transcript | NM_175924.3 |
| | | Variant 2 | |
| SEQ ID NO. 382 | ILDR1 | Amino Acid | NP_787120.1 |
| | | Sequence Isoform 2 | |
| | | Precursor | |
| SEQ ID NO. 383 | PDIA3 | mRNA | NM_005313.4 |
| SEQ ID NO. 384 | AIMP1 | mRNA Transcript | NM_004757.3 |
| | | Variant 1 | |
| SEQ ID NO. 385 | AIMP1 | mRNA Transcript | NM_001142415.1 |
| | | Variant 2 | |
| SEQ ID NO. 386 | AIMP1 | mRNA Transcript | NM_001142416.1 |
| | | Variant 3 | |
| SEQ ID NO. 387 | AIMP1 | mRNA Transcript | XM_017008835.1 |
| | | Variant X1 | |
| SEQ ID NO. 388 | AIMP1 | mRNA Transcript | XM_017008836.1 |
| | | Variant X2 | |
| SEQ ID NO. 389 | GPR63 | mRNA Transcript | NM_030784.3 |
| | | Variant 2 | |
| SEQ ID NO. 390 | GPR63 | mRNA Transcript | NM_001143957.2 |
| | | Variant 1 | |
| SEQ ID NO. 391 | GPR63 | mRNA Transcript | XM_011536153.2 |
| | | Variant X1 | |
| SEQ ID NO. 392 | GPR63 | mRNA Transcript | XM_017011334.1 |
| | | Variant X2 | |
| SEQ ID NO. 393 | GPR63 | mRNA Transcript | XM_011536154.2 |
| | | Variant X3 | |
| SEQ ID NO. 394 | GPR63 | mRNA Transcript | XM_011536155.2 |
| | | Variant X4 | |
| SEQ ID NO. 395 | GPR63 | mRNA Transcript | XM_011536157.2 |
| | | Variant X5 | |
| SEQ ID NO. 396 | GPR63 | mRNA Transcript | XM_011536159.2 |
| | | Variant X6 | |
| SEQ ID NO. 397 | GPR63 | mRNA Transcript | XM_006715570.3 |
| | | Variant X7 | |
| SEQ ID NO. 398 | CCR7 | mRNA Transcript | NM_001838.3 |
| | | Variant 1 | |
| SEQ ID NO. 399 | CCR7 | mRNA Transcript | NM_001301714.1 |
| | | Variant 2 | |
| SEQ ID NO. 400 | CCR7 | mRNA Transcript | NM_001301716.1 |
| | | Variant 3 | |
| SEQ ID NO. 401 | CCR7 | mRNA Transcript | NM_001301717.1 |
| | | Variant 4 | |
| SEQ ID NO. 402 | CCR7 | mRNA Transcript | NM_001301718.1 |
| | | Variant 5 | |
| SEQ ID NO. 403 | ATG9B | non-coding RNA | NR_073169.1 |
| | | Transcript Variant 2 | |
| SEQ ID NO. 404 | ATG9B | mRNA Transcript | XM_011516065.1 |
| | | Variant X1 | |
| SEQ ID NO. 405 | ATG9B | mRNA Transcript | NM_001317056.1 |
| | | Variant 3 | |
| SEQ ID NO. 406 | ATG9B | non-coding RNA | NR_133652.1 |
| | | Transcript Variant 1 | |
| SEQ ID NO. 407 | ATG9B | mRNA Transcript | XM_011516066.2 |
| | | Variant X2 | |
| SEQ ID NO. 408 | SLC9B1 | mRNA Transcript | NM_139173.3 |
| | | Variant 1 | |
| SEQ ID NO. 409 | SLC9B1 | mRNA Transcript | NM_001100874.2 |
| | | Variant 2 | |
| SEQ ID NO. 410 | SLC9B1 | non-coding RNA | NR_047513.1 |
| | | Transcript Variant 3 | |
| SEQ ID NO. 411 | SLC9B1 | non-coding RNA | NR_047515.1 |
| | | Transcript Variant 4 | |
| SEQ ID NO. 412 | SLC9B1 | mRNA Transcript | XM_011531622.1 |
| | | Variant X2 | |
| SEQ ID NO. 413 | SLC9B1 | mRNA Transcript | XM_011531623.1 |
| | | Variant X3 | |
| SEQ ID NO. 414 | SLC9B1 | mRNA Transcript | XM_006714093.3 |
| | | Variant X1 | |
| SEQ ID NO. 415 | CD99 | mRNA Transcript | NM_002414.4 |
| | | Variant 1 | |
| SEQ ID NO. 416 | CD99 | mRNA Transcript | NM_001122898.2 |
| | | Variant 2 | |
| SEQ ID NO. 417 | CD99 | mRNA Transcript | NM_001321367.1 |
| | | Variant 4 | |
| SEQ ID NO. 418 | CD99 | mRNA Transcript | NM_001321368.1 |
| | | Variant 5 | |
| SEQ ID NO. 419 | CD99 | mRNA Transcript | NM_001321369.1 |
| | | Variant 6 | |
| SEQ ID NO. 420 | CD99 | mRNA Transcript | NM_001321370.1 |
| | | Variant 7 | |
| SEQ ID NO. 421 | CD99 | non-coding RNA | NR_135623.1 |
| | | Transcript Variant 3 | |
| SEQ ID NO. 422 | LRP1 | mRNA | NM_002332.2 |
| SEQ ID NO. 423 | LRP1 | mRNA Transcript | XM_017019303.1 |
| | | Variant X1 | |
| SEQ ID NO. 424 | UBR4 | mRNA | NM_020765.2 |
| SEQ ID NO. 425 | UBR4 | mRNA Transcript | XM_011541108.2 |
| | | Variant X1 | |
| SEQ ID NO. 426 | UBR4 | mRNA Transcript | XM_011541109.2 |
| | | Variant X2 | |
| SEQ ID NO. 427 | UBR4 | mRNA Transcript | XM_011541110.2 |
| | | Variant X3 | |
| SEQ ID NO. 428 | UBR4 | mRNA Transcript | XM_011541111.2 |
| | | Variant X4 | |
| SEQ ID NO. 429 | UBR4 | mRNA Transcript | XM_011541112.2 |
| | | Variant X5 | |
| SEQ ID NO. 430 | UBR4 | mRNA Transcript | XM_011541113.2 |
| | | Variant X6 | |
| SEQ ID NO. 431 | UBR4 | mRNA Transcript | XM_011541114.2 |
| | | Variant X7 | |
| SEQ ID NO. 432 | UBR4 | mRNA Transcript | XM_017000822.1 |
| | | Variant X8 | |
| SEQ ID NO. 433 | UBR4 | mRNA Transcript | XM_011541115.2 |
| | | Variant X9 | |
| SEQ ID NO. 434 | UBR4 | mRNA Transcript | XM_011541116.2 |
| | | Variant X10 | |
| SEQ ID NO. 435 | UBR4 | mRNA Transcript | XM_017000823.1 |
| | | Variant X11 | |
| SEQ ID NO. 436 | UBR4 | mRNA Transcript | XM_011541117.2 |
| | | Variant X12 | |
| SEQ ID NO. 437 | UBR4 | mRNA Transcript | XM_011541118.2 |
| | | Variant X13 | |
| SEQ ID NO. 438 | UBR4 | mRNA Transcript | XM_017000824.1 |
| | | Variant X14 | |
| SEQ ID NO. 439 | UBR4 | mRNA Transcript | XM_017000825.1 |
| | | Variant X15 | |
| SEQ ID NO. 440 | UBR4 | mRNA Transcript | XM_017000826.1 |
| | | Variant X16 | |
| SEQ ID NO. 441 | UBR4 | mRNA Transcript | XM_011541119.2 |
| | | Variant X17 | |
| SEQ ID NO. 442 | UBR4 | mRNA Transcript | XM_011541120.2 |
| | | Variant X18 | |
| SEQ ID NO. 443 | UBR4 | mRNA Transcript | XM_017000827.1 |
| | | Variant X19 | |
| SEQ ID NO. 444 | UBR4 | mRNA Transcript | XM_011541121.2 |
| | | Variant X20 | |
| SEQ ID NO. 445 | UBR4 | mRNA Transcript | XM_017000828.1 |
| | | Variant X21 | |
| SEQ ID NO. 446 | UBR4 | mRNA Transcript | XM_017000829.1 |
| | | Variant X22 | |
| SEQ ID NO. 447 | UBR4 | mRNA Transcript | XM_017000830.1 |
| | | Variant X23 | |
| SEQ ID NO. 448 | ATP6AP2 | mRNA | NM_005765.2 |
| SEQ ID NO. 449 | TEX10 | mRNA Transcript | NM_017746.3 |
| | | Variant 1 | |
| SEQ ID NO. 450 | TEX10 | mRNA Transcript | NM_001161584.1 |
| | | Variant 2 | |
| SEQ ID NO. 451 | TEX10 | mRNA Transcript | XM_011518798.1 |
| | | Variant X1 | |
| SEQ ID NO. 452 | TEX10 | misc RNA Transcript | XR_929813.1 |
| | | Variant X2 | |
| SEQ ID NO. 453 | TEX10 | misc RNA Transcript | XR_001746327.1 |
| | | Variant X3 | |
| SEQ ID NO. 454 | TEX10 | mRNA Transcript | XM_017014848.1 |
| | | Variant X4 | |
| SEQ ID NO. 455 | CNGB1 | mRNA Transcript | NM_001297.4 |
| | | Variant 1 | |
| SEQ ID NO. 456 | CNGB1 | mRNA Transcript | NM_001135639.1 |
| | | Variant 2 | |
| SEQ ID NO. 457 | CNGB1 | mRNA Transcript | NM_001286130.1 |
| | | Variant 3 | |
| SEQ ID NO. 458 | CNGB1 | mRNA Transcript | XM_011522870.2 |
| | | Variant X1 | |
| SEQ ID NO. 459 | SPN | mRNA Transcript | NM_001030288.3 |
| | | Variant 1 | |
| SEQ ID NO. 460 | SPN | mRNA Transcript | NM_003123.5 |
| | | Variant 2 | |
| SEQ ID NO. 461 | PILRB | mRNA | NM_178238.3 |
| SEQ ID NO. 462 | JAM2 | mRNA Transcript | NM_021219.3 |
| | | Variant 1 | |
| SEQ ID NO. 463 | JAM2 | mRNA Transcript | NM_001270407.1 |
| | | Variant 2 | |
| SEQ ID NO. 464 | JAM2 | mRNA Transcript | NM_001270408.1 |
| | | Variant 3 | |
| SEQ ID NO. 465 | JAM2 | non-coding RNA | NR_072999.1 |
| | | Transcript Variant 4 | |
| SEQ ID NO. 466 | JAM2 | misc RNA Transcript | XR_937537.2 |
| | | Variant X1 | |
| SEQ ID NO. 467 | PDGFA | mRNA Transcript | NM_002607.5 |
| | | Variant 1 | |
| SEQ ID NO. 468 | PDGFA | mRNA Transcript | NM_033023.4 |
| | | Variant 2 | |
| SEQ ID NO. 469 | PDGFA | mRNA Transcript | XM_011515415.1 |
| | | Variant X1 | |
| SEQ ID NO. 470 | PDGFA | mRNA Transcript | XM_011515416.1 |
| | | Variant X2 | |
| SEQ ID NO. 471 | PDGFA | mRNA Transcript | XM_011515417.1 |
| | | Variant X3 | |
| SEQ ID NO. 472 | PDGFA | mRNA Transcript | XM_011515418.1 |
| | | Variant X4 | |
| SEQ ID NO. 473 | PDGFA | mRNA Transcript | XM_017012289.1 |
| | | Variant X5 | |
| SEQ ID NO. 474 | PDGFA | mRNA Transcript | XM_011515419.2 |
| | | Variant X6 | |
| SEQ ID NO. 475 | CD46 | mRNA Transcript | NM_002389.4 |
| | | Variant a | |
| SEQ ID NO. 476 | CD46 | mRNA Transcript | NM_172359.2 |
| | | Variant b | |
| SEQ ID NO. 477 | CD46 | mRNA Transcript | NM_172351.2 |
| | | Variant c | |
| SEQ ID NO. 478 | CD46 | mRNA Transcript | NM_153826.3 |
| | | Variant d | |
| SEQ ID NO. 479 | CD46 | mRNA Transcript | NM_172352.2 |
| | | Variant e | |
| SEQ ID NO. 480 | CD46 | mRNA Transcript | NM_172353.2 |
| | | Variant f | |
| SEQ ID NO. 481 | CD46 | mRNA Transcript | NM_172361.2 |
| | | Variant l | |
| SEQ ID NO. 482 | CD46 | mRNA Transcript | NM_172350.2 |
| | | Variant n | |
| SEQ ID NO. 483 | CD46 | mRNA Transcript | XM_011509563.1 |
| | | Variant X1 | |
| SEQ ID NO. 484 | CD46 | mRNA Transcript | XM_011509564.1 |
| | | Variant X3 | |
| SEQ ID NO. 485 | CD46 | mRNA Transcript | XM_017001308.1 |
| | | Variant X2 | |
| SEQ ID NO. 486 | CD46 | mRNA Transcript | XM_017001309.1 |
| | | Variant X4 | |
| SEQ ID NO. 487 | CD46 | mRNA Transcript | XM_017001310.1 |
| | | Variant X5 | |
| SEQ ID NO. 488 | CD46 | misc RNA Transcript | XR_001737177.1 |
| | | Variant X6 | |
| SEQ ID NO. 489 | CD46 | misc RNA Transcript | XR_001737178.1 |
| | | Variant X7 | |
| SEQ ID NO. 490 | NDUFB1 | mRNA | NM_004545.3 |
| SEQ ID NO. 491 | GYPE | mRNA Transcript | NM_198682.2 |
| | | Variant 2 | |
| SEQ ID NO. 492 | GYPE | mRNA Transcript | NM_002102.3 |
| | | Variant 1 | |
| SEQ ID NO. 493 | GYPE | mRNA Transcript | XM_017008139.1 |
| | | Variant X1 | |
| SEQ ID NO. 494 | GYPE | mRNA Transcript | XM_017008140.1 |
| | | Variant X2 | |
| SEQ ID NO. 495 | SLC12A8 | mRNA Transcript | NM_024628.5 |
| | | Variant 1 | |
| SEQ ID NO. 496 | SLC12A8 | mRNA Transcript | NM_001195483.1 |
| | | Variant 2 | |
| SEQ ID NO. 497 | SLC2A14 | mRNA Transcript | NM_001286234.1 |
| | | Variant 3 | |
| SEQ ID NO. 498 | SLC2A14 | mRNA Transcript | NM_001286233.1 |
| | | Variant 1 | |
| SEQ ID NO. 499 | SLC2A14 | mRNA Transcript | NM_001286236.1 |
| | | Variant 5 | |
| SEQ ID NO. 500 | SLC2A14 | mRNA Transcript | NM_001286235.1 |
| | | Variant 4 | |
| SEQ ID NO. 501 | SLC2A14 | mRNA Transcript | NM_153449.3 |
| | | Variant 2 | |
| SEQ ID NO. 502 | SLC2A14 | mRNA Transcript | NM_001286237.1 |
| | | Variant 6 | |
| SEQ ID NO. 503 | SLC2A14 | mRNA Transcript | XM_011520562.1 |
| | | Variant X6 | |
| SEQ ID NO. 504 | SLC2A14 | mRNA Transcript | XM_017018841.1 |
| | | Variant X1 | |
| SEQ ID NO. 505 | SLC2A14 | mRNA Transcript | XM_017018842.1 |
| | | Variant X2 | |
| SEQ ID NO. 506 | SLC2A14 | mRNA Transcript | XM_017018843.1 |
| | | Variant X3 | |
| SEQ ID NO. 507 | SLC2A14 | mRNA Transcript | XM_017018844.1 |
| | | Variant X4 | |
| SEQ ID NO. 508 | SLC2A14 | mRNA Transcript | XM_017018845.1 |
| | | Variant X5 | |
| SEQ ID NO. 509 | SLC2A14 | mRNA Transcript | XM_017018846.1 |
| | | Variant X7 | |
| SEQ ID NO. 510 | SLC2A14 | mRNA Transcript | XM_005253315.3 |
| | | Variant X8 | |
| SEQ ID NO. 511 | SLC2A14 | mRNA Transcript | XM_005253317.4 |
| | | Variant X9 | |
| SEQ ID NO. 512 | SLC2A14 | mRNA Transcript | XM_017018847.1 |
| | | Variant X10 | |
| SEQ ID NO. 513 | SLC2A14 | mRNA Transcript | XM_011520563.2 |
| | | Variant X11 | |
| SEQ ID NO. 514 | SLC2A14 | mRNA Transcript | XM_011520564.2 |
| | | Variant X12 | |
| SEQ ID NO. 515 | SLC2A14 | mRNA Transcript | XM_011520565.2 |
| | | Variant X13 | |
| SEQ ID NO. 516 | RNF19B | mRNA Transcript | NM_153341.3 |
| | | Variant 1 | |
| SEQ ID NO. 517 | RNF19B | mRNA Transcript | NM_001127361.2 |
| | | Variant 2 | |
| SEQ ID NO. 518 | RNF19B | mRNA Transcript | NM_001300826.1 |
| | | Variant 3 | |
| SEQ ID NO. 519 | RNF19B | mRNA Transcript | XM_006710356.2 |
| | | Variant X1 | |
| SEQ ID NO. 520 | RNF19B | mRNA Transcript | XM_006710357.3 |
| | | Variant X2 | |
| SEQ ID NO. 521 | RNF19B | mRNA Transcript | XM_006710358.3 |
| | | Variant X3 | |
| SEQ ID NO. 522 | SPCS1 | mRNA | NM_014041.3 |
| SEQ ID NO. 523 | SLC35F6 | mRNA | NM_017877.3 |
| SEQ ID NO. 524 | CD36 | mRNA Transcript | NM_001001548.2 |
| | | Variant 1 | |
| SEQ ID NO. 525 | CD36 | mRNA Transcript | NM_001001547.2 |
| | | Variant 2 | |
| SEQ ID NO. 526 | CD36 | mRNA Transcript | NM_000072.3 |
| | | Variant 3 | |
| SEQ ID NO. 527 | CD36 | mRNA Transcript | NM_001127443.1 |
| | | Variant 4 | |
| SEQ ID NO. 528 | CD36 | mRNA Transcript | NM_001127444.1 |
| | | Variant 5 | |
| SEQ ID NO. 529 | CD36 | mRNA Transcript | XM_005250713.1 |
| | | Variant X2 | |
| SEQ ID NO. 530 | CD36 | mRNA Transcript | XM_005250714.1 |
| | | Variant X3 | |
| SEQ ID NO. 531 | CD36 | mRNA Transcript | NM_001289908.1 |
| | | Variant 6 | |
| SEQ ID NO. 532 | CD36 | mRNA Transcript | NM_001289909.1 |
| | | Variant 7 | |
| SEQ ID NO. 533 | CD36 | mRNA Transcript | NM_001289911.1 |
| | | Variant 8 | |
| SEQ ID NO. 534 | CD36 | non-coding RNA | NR_110501.1 |
| | | Transcript Variant 9 | |
| SEQ ID NO. 535 | CD36 | mRNA Transcript | XM_005250715.4 |
| | | Variant X1 | |
| SEQ ID NO. 536 | ITM2B | mRNA | NM_021999.4 |
| SEQ ID NO. 537 | GLG1 | mRNA Transcript | NM_.012201.5 |
| | | Variant 1 | |
| SEQ ID NO. 538 | GLG1 | mRNA Transcript | NM_001145666.1 |
| | | Variant 2 | |
| SEQ ID NO. 539 | GLG1 | mRNA Transcript | NM_001145667.1 |
| | | Variant 3 | |
| SEQ ID NO. 540 | GLG1 | non-coding RNA | NR_027264.1 |
| | | Transcript Variant 4 | |
| SEQ ID NO. 541 | GLG1 | non-coding RNA | NR_027265.1 |
| | | Transcript Variant 5 | |
| SEQ ID NO. 542 | SMIM24 | mRNA | NM_001136503.1 |
| SEQ ID NO. 543 | TMEM50A | mRNA | NM_014313.3 |
| SEQ ID NO. 544 | TMEM50A | mRNA Transcript | XM_005245817.1 |
| | | Variant X2 | |
| SEQ ID NO. 545 | TMEM50A | mRNA Transcript | XM_011541159.1 |
| | | Variant X1 | |
| SEQ ID NO. 546 | HSPA5 | mRNA | NM_005347.4 |
| SEQ ID NO. 547 | ITGAX | mRNA Transcript | NM_001286375.1 |
| | | Variant 1 | |
| SEQ ID NO. 548 | ITGAX | mRNA Transcript | NM_000887.4 |
| | | Variant 2 | |
| SEQ ID NO. 549 | ITGAX | mRNA Transcript | XM_011545852.1 |
| | | Variant X2 | |
| SEQ ID NO. 550 | ITGAX | mRNA Transcript | XM_011545854.1 |
| | | Variant X3 | |
| SEQ ID NO. 551 | ITGAX | misc RNA Transcript | XR_950797.2 |
| | | Variant X1 | |
| SEQ ID NO. 552 | SLC24A2 | mRNA Transcript | NM_020344.3 |
| | | Variant 1 | |
| SEQ ID NO. 553 | SLC24A2 | mRNA Transcript | NM_001193288.2 |
| | | Variant 2 | |
| SEQ ID NO. 554 | SLC24A2 | mRNA Transcript | XM_005251426.4 |
| | | Variant X1 | |
| SEQ ID NO. 555 | SLC24A2 | mRNA Transcript | XM_005251425.2 |
| | | Variant X2 | |
| SEQ ID NO. 556 | SLC24A2 | mRNA Transcript | XM_017014592.1 |
| | | Variant X3 | |
| SEQ ID NO. 557 | SLC24A2 | mRNA Transcript | XM_006716750.3 |
| | | Variant X4 | |
| SEQ ID NO. 558 | SLC24A2 | mRNA Transcript | XM_017014593.1 |
| | | Variant X5 | |
| SEQ ID NO. 559 | SLC2A3 | mRNA | NM_006931.2 |
| SEQ ID NO. 560 | RAB11FIP3 | mRNA Transcript | NM_014700.3 |
| | | Variant 1 | |
| SEQ ID NO. 561 | RAB11FIP3 | mRNA Transcript | NM_001142272.1 |
| | | Variant 2 | |
| SEQ ID NO. 562 | RAB11FIP3 | mRNA Transcript | XM_005255714.1 |
| | | Variant X3 | |
| SEQ ID NO. 563 | RAB11FIP3 | mRNA Transcript | XM_005255713.3 |
| | | Variant X1 | |
| SEQ ID NO. 564 | RAB11FIP3 | mRNA Transcript | XM_017023907.1 |
| | | Variant X2 | |
| SEQ ID NO. 565 | RAB11FIP3 | mRNA Transcript | XM_005255715.4 |
| | | Variant X4 | |
| SEQ ID NO. 566 | RAB11FIP3 | mRNA Transcript | XM_011522764.2 |
| | | Variant X5 | |
| SEQ ID NO. 567 | RAB11FIP3 | mRNA Transcript | XM_005255717.3 |
| | | Variant X6 | |
| SEQ ID NO. 568 | RAB11FIP3 | mRNA Transcript | XM_011522765.2 |
| | | Variant X7 | |
| SEQ ID NO. 569 | RAB11FIP3 | mRNA Transcript | XM_005255718.3 |
| | | Variant X8 | |
| SEQ ID NO. 570 | RAB11FIP3 | misc RNA Transcript | XR_932981.2 |
| | | Variant X9 | |
| SEQ ID NO. 571 | IL18R1 | mRNA Transcript | NM_003855.3 |
| | | Variant 1 | |
| SEQ ID NO. 572 | IL18R1 | mRNA Transcript | NM_001282399.1 |
| | | Variant 2 | |
| SEQ ID NO. 573 | IL18R1 | mRNA Transcript | XM_005264039.3 |
| | | Variant X1 | |
| SEQ ID NO. 574 | IL18R1 | mRNA Transcript | XM_005264040.3 |
| | | Variant X2 | |
| SEQ ID NO. 575 | IL18R1 | mRNA Transcript | XM_011512099.1 |
| | | Variant X3 | |
| SEQ ID NO. 576 | IL18R1 | mRNA Transcript | XM_017005181.1 |
| | | Variant X4 | |
| SEQ ID NO. 577 | IL18R1 | misc RNA Transcript | XR_923054.2 |
| | | Variant X5 | |
| SEQ ID NO. 578 | IL18R1 | mRNA Transcript | XM_017005182.1 |
| | | Variant X6 | |
| SEQ ID NO. 579 | IL18R1 | mRNA Transcript | XM_017005183.1 |
| | | Variant X7 | |
| SEQ ID NO. 580 | IL18R1 | mRNA Transcript | XM_017005184.1 |
| | | Variant X8 | |
| SEQ ID NO. 581 | CCDC47 | mRNA | NM_020198.2 |
| SEQ ID NO. 582 | CCDC47 | mRNA Transcript | XM_005257527.2 |
| | | Variant X1 | |
| SEQ ID NO. 583 | FZD4 | mRNA | NM_012193.3 |
| SEQ ID NO. 584 | SHISA9 | mRNA Transcript | NM_001145205.1 |
| | | Variant 2 | |
| SEQ ID NO. 585 | SHISA9 | mRNA Transcript | NM_001145204.2 |
| | | Variant 1 | |
| SEQ ID NO. 586 | SHISA9 | mRNA Transcript | XM_005255539.3 |
| | | Variant X1 | |
| SEQ ID NO. 587 | SHISA9 | mRNA Transcript | XM_011522642.2 |
| | | Variant X2 | |
| SEQ ID NO. 588 | SHISA9 | misc RNA Transcript | XR_932915.2 |
| | | Variant X3 | |
| SEQ ID NO. 589 | SHISA9 | misc RNA Transcript | XR_001751975.1 |
| | | Variant X4 | |
| SEQ ID NO. 590 | SHISA9 | misc RNA Transcript | XR_001751976.1 |
| | | Variant X5 | |
| SEQ ID NO. 591 | SORCS1 | mRNA Transcript | NM_001206570.1 |
| | | Variant 4 | |
| SEQ ID NO. 592 | SORCS1 | mRNA Transcript | NM_001206571.1 |
| | | Variant 5 | |
| SEQ ID NO. 593 | SORCS1 | mRNA Transcript | NM_001206572.1 |
| | | Variant 6 | |
| SEQ ID NO. 594 | SORCS1 | mRNA Transcript | NM_001013031.2 |
| | | Variant 2 | |
| SEQ ID NO. 595 | SORCS1 | mRNA Transcript | NM_052918.4 |
| | | Variant 1 | |
| SEQ ID NO. 596 | SORCS1 | mRNA Transcript | NM_001206569.1 |
| | | Variant 3 | |
| SEQ ID NO. 597 | SORCS1 | mRNA Transcript | XM_017015614.1 |
| | | Variant X1 | |
| SEQ ID NO. 598 | SORCS1 | mRNA Transcript | XM_011539199.2 |
| | | Variant X2 | |
| SEQ ID NO. 599 | SORCS1 | mRNA Transcript | XM_017015615.1 |
| | | Variant X3 | |
| SEQ ID NO. 600 | SORCS1 | mRNA Transcript | XM_011539201.2 |
| | | Variant X4 | |
| SEQ ID NO. 601 | SORCS1 | mRNA Transcript | XM_017015616.1 |
| | | Variant X5 | |
| SEQ ID NO. 602 | SORCS1 | mRNA Transcript | XM_017015617.1 |
| | | Variant X6 | |
| SEQ ID NO. 603 | SORCS1 | mRNA Transcript | XM_017015618.1 |
| | | Variant X7 | |
| SEQ ID NO. 604 | CLIC4 | mRNA | NM_013943.2 |
| SEQ ID NO. 605 | MS4A2 | cDNA Transcript | NM_000139.4 |
| | | Variant 1 | |
| SEQ ID NO. 606 | MS4A2 | Amino Acid | NP_000130.1 |
| | | Sequence Isoform 1 | |
| SEQ ID NO. 607 | MS4A2 | cDNA Transcript | NM_001256916.1 |
| | | Variant 3 | |
| SEQ ID NO. 608 | MS4A2 | Amino Acid | NP_001243845.1 |
| | | Sequence Isoform 3 | |
| SEQ ID NO. 609 | MLNR | cDNA | NM_001507.1 |
| SEQ ID NO. 610 | MLNR | Amino Acid | NP_001498.1 |
| | | Sequence | |
| SEQ ID NO. 611 | TIGIT | cDNA | NM_173799.3 |
| SEQ ID NO. 612 | TIGIT | Amino Acid | NP_776160.2 |
| | | Sequence | |
| SEQ ID NO. 613 | CNGA1 | cDNA Transcript | NM_001142564.1 |
| | | Variant 1 | |
| SEQ ID NO. 614 | CNGA1 | Amino Acid | NP_001136036.1 |
| | | Sequence Isoform 1 | |
| SEQ ID NO. 615 | CNGA1 | cDNA Transcript | NM_000087.3 |
| | | Variant 2 | |
| SEQ ID NO. 616 | CNGA1 | Amino Acid | NP_000078.2 |
| | | Sequence Isoform 2 | |
| SEQ ID NO. 617 | SIRPB2 | cDNA Transcript | NM_001122962.1 |
| | | Variant 1 | |
| SEQ ID NO. 618 | SIRPB2 | Amino Acid | NP_001116434.1 |
| | | Sequence Isoform 1 | |
| | | Precursor | |
| SEQ ID NO. 619 | SIRPB2 | cDNA Transcript | NM_001134836.1 |
| | | Variant 2 | |
| SEQ ID NO. 620 | SIRPB2 | Amino Acid | NP_001128308.1 |
| | | Sequence Isoform 2 | |
| | | Precursor | |
| SEQ ID NO. 621 | PRRG4 | cDNA | NM_024081.5 |
| SEQ ID NO. 622 | PRRG4 | Amino Acid | NP_076986.1 |
| | | Sequence | |
| SEQ ID NO. 623 | VSTM4 | cDNA Transcript | NM_001031746.4 |
| | | Variant 1 | |
| SEQ ID NO. 624 | VSTM4 | Amino Acid | NP_001026916.2 |
| | | Sequence Isoform 1 | |
| | | Precursor | |
| SEQ ID NO. 625 | VSTM4 | cDNA Transcript | NM_144984.3 |
| | | Variant 2 | |
| SEQ ID NO. 626 | VSTM4 | Amino Acid | NP_659421.1 |
| | | Sequence Isoform 2 | |
| | | Precursor | |
| SEQ ID NO. 627 | TMEM107 | cDNA Transcript | NM_032354.4 |
| | | Variant 1 | |
| SEQ ID NO. 628 | TMEM107 | Amino Acid | NP_115730.2 |
| | | Sequence Isoform 1 | |
| SEQ ID NO. 629 | TMEM107 | cDNA Transcript | NM_183065.3 |
| | | Variant 2 | |
| SEQ ID NO. 630 | TMEM107 | Amino Acid | NP_898888.1 |
| | | Sequence isoform 2 | |
| SEQ ID NO. 631 | TMEM107 | cDNA Transcript | NM_001351278.1 |
| | | Variant 3 | |
| SEQ ID NO. 632 | TMEM107 | Amino Acid | NP_001338207.1 |
| | | Sequence isoform 3 | |
| SEQ ID NO. 633 | TMEM107 | cDNA Transcript | NM_001351279.1 |
| | | Variant 4 | |
| SEQ ID NO. 634 | TMEM107 | Amino Acid | NP_001338208.1 |
| | | Sequence Isoform 4 | |
| SEQ ID NO. 635 | TMEM107 | cDNA Transcript | NM_001351280.1 |
| | | Variant 5 | |
| SEQ ID NO. 636 | TMEM107 | Amino Acid | NP_001338209.1 |
| | | Sequence isoform 5 | |
| SEQ ID NO. 637 | NETO2 | cDNA Transcript | NM_018092.4 |
| | | Variant 1 | |
| SEQ ID NO. 638 | NETO2 | Amino Acid | NP_060562.3 |
| | | Sequence isoform 1 | |
| | | Precursor | |
| SEQ ID NO. 639 | NETO2 | cDNA Transcript | NM_001201477.1 |
| | | Variant 2 | |
| SEQ ID NO. 640 | NETO2 | Amino Acid | NP_001188406.1 |
| | | Sequence Isoform 2 | |
| | | Precursor | |
| SEQ ID NO. 641 | CSF1R | cDNA Transcript | NM_005211.3 |
| | | Variant 1 | |
| SEQ ID NO. 642 | CSF1R | Amino Acid | NP_005202.2 |
| | | Sequence | |
| | | Precursor | |
| SEQ ID NO. 643 | CSF1R | cDNA Transcript | NM_001288705.2 |
| | | Variant 2 | |
| SEQ ID NO. 644 | CSF1R | Amino Acid | NP_001275634.1 |
| | | Sequence | |
| | | Precursor | |
| SEQ ID NO. 645 | CSF1R | cDNA Transcript | NM_001349736.1 |
| | | Variant 4 | |
| SEQ ID NO. 646 | CSF1R | Amino Acid | NP_001336665.1 |
| | | Sequence | |
| | | Precursor | |
| SEQ ID NO. 647 | ADRB2 | cDNA | NM_000024.5 |
| SEQ ID NO. 648 | ADRB2 | Amino Acid | NP_000015.1 |
| | | Sequence | |
| SEQ ID NO. 649 | TLR2 | cDNA Transcript | NM_001318787.1 |
| | | Variant 1 | |
| SEQ ID NO. 650 | TLR2 | Amino Acid | NP_001305716.1 |
| | | Sequence | |
| | | Precursor | |
| SEQ ID NO. 651 | TLR2 | cDNA Transcript | NM_001318789.1 |
| | | Variant 2 | |
| SEQ ID NO. 652 | TLR2 | Amino Acid | NP_001305718.1 |
| | | Sequence | |
| | | Precursor | |
| SEQ ID NO. 653 | TLR2 | cDNA Transcript | NM_003264.4 |
| | | Variant 3 | |
| SEQ ID NO. 654 | TLR2 | Amino Acid | NP_003255.2 |
| | | Sequence | |
| | | Precursor | |
| SEQ ID NO. 655 | TLR2 | cDNA Transcript | NM_001318790.1 |
| | | Variant 4 | |
| SEQ ID NO. 656 | TLR2 | Amino Acid | NP_001305719.1 |
| | | Sequence | |
| | | Precursor | |
| SEQ ID NO. 657 | TLR2 | cDNA Transcript | NM_001318791.1 |
| | | Variant 5 | |
| SEQ ID NO. 658 | TLR2 | Amino Acid | NP_001305720.1 |
| | | Sequence | |
| | | Precursor | |
| SEQ ID NO. 659 | TLR2 | cDNA Transcript | NM_001318793.1 |
| | | Variant 6 | |
| SEQ ID NO. 660 | TLR2 | Amino Acid | NP_001305722.1 |
| | | Sequence | |
| | | Precursor | |
| SEQ ID NO. 661 | TLR2 | cDNA Transcript | NM_001318795.1 |
| | | Variant 7 | |
| SEQ ID NO. 662 | TLR2 | Amino Acid | NP_001305724.1 |
| | | Sequence | |
| | | Precursor | |
| SEQ ID NO. 663 | TLR2 | cDNA Transcript | NM_001318796.1 |
| | | Variant 8 | |
| SEQ ID NO. 664 | TLR2 | Amino Acid | NP_001305725.1 |
| | | Sequence | |
| | | Precursor | |
| SEQ ID NO. 665 | FUT4 | cDNA | NM_002033.3 |
| SEQ ID NO. 666 | FUT4 | Amino Acid | NP_002024.1 |
| | | Sequence | |
| SEQ ID NO. 667 | MGST1 | cDNA Transcript | NM_145792.2 |
| | | Variant 1 | |
| SEQ ID NO. 668 | MGST1 | Amino Acid | NP_665735.1 |
| | | Sequence isoform a | |
| SEQ ID NO. 669 | MGST1 | cDNA Transcript | NM_020300.4 |
| | | Variant 2 | |
| SEQ ID NO. 670 | MGST1 | Amino Acid | NP_064696.1 |
| | | Sequence Isoform a | |
| SEQ ID NO. 671 | MGST1 | cDNA Transcript | NM_145791.2 |
| | | Variant 3 | |
| SEQ ID NO. 672 | MGST1 | Amino Acid | NP_665734.1 |
| | | Sequence Isoform a | |
| SEQ ID NO. 673 | MGST1 | cDNA Transcript | NM_145764.2 |
| | | Variant 4 | |
| SEQ ID NO. 674 | MGST1 | Amino Acid | NP_665707.1 |
| | | Sequence Isoform a | |
| SEQ ID NO. 675 | MGST1 | cDNA Transcript | NM_001260511.1 |
| | | Variant 5 | |
| SEQ ID NO. 676 | MGST1 | Amino Acid | NP_001247440.1 |
| | | Sequence Isoform a | |
| SEQ ID NO. 677 | MGST1 | cDNA Transcript | NM_001260512.1 |
| | | Variant 6 | |
| SEQ ID NO. 678 | MGST1 | Amino Acid | NP_001247441.1 |
| | | Sequence Isoform b | |
| SEQ ID NO. 679 | MGST1 | cDNA Transcript | NM_001267598.1 |
| | | Variant 8 | |
| SEQ ID NO. 680 | MGST1 | Amino Acid | NP_001254527.1 |
| | | Sequence Isoform c | |
| SEQ ID NO. 681 | CSF3R | cDNA Transcript | XM_005270493.1 |
| | | Variant X4 | |
| SEQ ID NO. 682 | CSF3R | Amino Acid | XP_005270550.1 |
| | | Sequence Isoform X3 | |
| SEQ ID NO. 683 | CSF3R | cDNA Transcript | XM_011540749.1 |
| | | Variant X3 | |
| SEQ ID NO. 684 | CSF3R | Amino Acid | XP_011539051.1 |
| | | Sequence Isoform X2 | |
| SEQ ID NO. 685 | CSF3R | cDNA Transcript | XM_017000370.1 |
| | | Variant X1 | |
| SEQ ID NO. 686 | CSF3R | Amino Acid | XP_016855859.1 |
| | | Sequence Isoform X1 | |
| SEQ ID NO. 687 | CSF3R | cDNA Transcript | XM_011540748.2 |
| | | Variant X2 | |
| SEQ ID NO. 688 | CSF3R | Amino Acid | XP_011539050.1 |
| | | Sequence Isoform X1 | |
| SEQ ID NO. 689 | CSF3R | cDNA Transcript | XM_011540750.1 |
| | | Variant X5 | |
| SEQ ID NO. 690 | CSF3R | Amino Acid | XP_011539052.1 |
| | | Sequence Isoform X4 | |
| SEQ ID NO. 691 | HLA-B | cDNA | NM_005514.7 |
| SEQ ID NO. 692 | HLA-B | Amino Acid | NP_005505.2 |
| | | Sequence | |
| | | Precursor | |
| SEQ ID NO. 693 | ITGA10 | cDNA Transcript | NM_003637.4 |
| | | Variant 1 | |
| SEQ ID NO. 694 | ITGA10 | Amino Acid | NP_003628.2 |
| | | Sequence Isoform 1 | |
| | | Precursor | |
| SEQ ID NO. 695 | ITGA10 | cDNA Transcript | NM_001303040.1 |
| | | Variant 2 | |
| SEQ ID NO. 696 | ITGA10 | Amino Acid | NP_001289969.1 |
| | | Sequence Isoform 2 | |
| SEQ ID NO. 697 | ITGA10 | cDNA Transcript | NM_001303041.1 |
| | | Variant 3 | |
| SEQ ID NO. 698 | ITGA10 | Amino Acid | NP_001289970.1 |
| | | Sequence Isoform 3 | |
| | | Precursor | |
| SEQ ID NO. 699 | SLC26A8 | cDNA Transcript | NM_052961.3 |
| | | Variant 1 | |
| SEQ ID NO. 700 | SLC26A8 | Amino Acid | NP_443193.1 |
| | | Sequence Isoform a | |
| SEQ ID NO. 701 | SLC26A8 | cDNA Transcript | NM_138718.2 |
| | | Variant 2 | |
| SEQ ID NO. 702 | SLC26A8 | Amino Acid | NP_619732.2 |
| | | Sequence Isoform b | |
| SEQ ID NO. 703 | SLC26A8 | cDNA Transcript | NM_001193476.1 |
| | | Variant 3 | |
| SEQ ID NO. 704 | SLC26A8 | Amino Acid | NP_001180405.1 |
| | | Sequence Isoform a | |
| SEQ ID NO. 705 | SIRPB1 | cDNA Transcript | NM_001083910.3 |
| | | Variant 2 | |
| SEQ ID NO. 706 | SIRPB1 | Amino Acid | NP_001077379.1 |
| | | Sequence isoform 2 | |
| | | Precursor | |
| SEQ ID NO. 707 | SIRPB1 | cDNA Transcript | NM_001135844.3 |
| | | Variant 3 | |
| SEQ ID NO. 708 | SIRPB1 | Amino Acid | NP_001129316.1 |
| | | Sequence Isoform 3 | |
| | | Precursor | |
| SEQ ID NO. 709 | SIRPB1 | cDNA Transcript | NM_001329157.1 |
| | | Variant 4 | |
| SEQ ID NO. 710 | SIRPB1 | Amino Acid | NP_001316086.1 |
| | | Sequence Isoform 3 | |
| | | Precursor | |
| SEQ ID NO. 711 | SIRPB1 | cDNA Transcript | NM_001330639.1 |
| | | Variant 5 | |
| SEQ ID NO. 712 | SIRPB1 | Amino Acid | NP_001317568.1 |
| | | Sequence Isoform 4 | |
| | | Precursor | |
| SEQ ID NO. 713 | SIRPB1 | cDNA Transcript | NM_006065.4 |
| | | Variant 1 | |
| SEQ ID NO. 714 | SIRPB1 | Amino Acid | NP_006056.2 |
| | | Sequence isoform 1 | |
| | | Precursor | |
| SEQ ID NO. 715 | RAET1E | cDNA Transcript | NM_139165.2 |
| | | Variant 1 | |
| SEQ ID NO. 716 | RAET1E | Amino Acid | NP_631904.1 |
| | | Sequence Isoform 1 | |
| | | Precursor | |
| SEQ ID NO. 717 | RAET1E | cDNA Transcript | NM_001243325.1 |
| | | Variant 2 | |
| SEQ ID NO. 718 | RAET1E | Amino Acid | NP_001230254.1 |
| | | Sequence Isoform 2 | |
| | | Precursor | |
| SEQ ID NO. 719 | RAET1E | cDNA Transcript | NM_001243327.1 |
| | | Variant 3 | |
| SEQ ID NO. 720 | RAET1E | Amino Acid | NP_001230256.1 |
| | | Sequence Isoform 3 | |
| | | Precursor | |
| SEQ ID NO. 721 | RAET1E | cDNA Transcript | NM_001243328.1 |
| | | Variant 4 | |
| SEQ ID NO. 722 | RAET1E | Amino Acid | NP_001230257.1 |
| | | Sequence Isoform 4 | |
| | | Precursor | |
| SEQ ID NO. 723 | ST3GAL6 | cDNA Transcript | NM_001271142.1 |
| | | Variant 2 | |
| SEQ ID NO. 724 | ST3GAL6 | Amino Acid | NP_001258071.1 |
| | | Sequence Isoform 2 | |
| SEQ ID NO. 725 | ST3GAL6 | cDNA Transcript | NM_001271145.1 |
| | | Variant 3 | |
| SEQ ID NO. 726 | ST3GAL6 | Amino Acid | NP_001258074.1 |
| | | Sequence Isoform 3 | |
| SEQ ID NO. 727 | ST3GAL6 | cDNA Transcript | NM_001271146.1 |
| | | Variant 4 | |
| SEQ ID NO. 728 | ST3GAL6 | Amino Acid | NP_001258075.1 |
| | | Sequence Isoform 1 | |
| SEQ ID NO. 729 | ST3GAL6 | cDNA Transcript | NM_001271147.1 |
| | | Variant 5 | |
| SEQ ID NO. 730 | ST3GAL6 | Amino Acid | NP_001258076.1 |
| | | Sequence Isoform 4 | |
| SEQ ID NO. 731 | ST3GAL6 | cDNA Transcript | NM_001271148.1 |
| | | Variant 6 | |
| SEQ ID NO. 732 | ST3GAL6 | Amino Acid | NP_001258077.1 |
| | | Sequence Isoform 5 | |
| SEQ ID NO. 733 | ST3GAL6 | cDNA Transcript | NM_001323352.1 |
| | | Variant 7 | |
| SEQ ID NO. 734 | ST3GAL6 | Amino Acid | NP_ 001310281.1 |
| | | Sequence Isoform 1 | |
| SEQ ID NO. 735 | ST3GAL6 | cDNA Transcript | NM_001323353.1 |
| | | Variant 15 | |
| SEQ ID NO. 736 | ST3GAL6 | Amino Acid | NP_001310282.1 |
| | | Sequence Isoform 5 | |
| SEQ ID NO. 737 | ST3GAL6 | cDNA Transcript | NM_001323358.1 |
| | | Variant 17 | |
| SEQ ID NO. 738 | ST3GAL6 | Amino Acid | NP_001310287.1 |
| | | Sequence Isoform 7 | |
| SEQ ID NO. 739 | ST3GAL6 | cDNA Transcript | NM_001323359.1 |
| | | Variant 18 | |
| SEQ ID NO. 740 | ST3GAL6 | Amino Acid | NP_001310288.1 |
| | | Sequence Isoform 7 | |
| SEQ ID NO. 741 | ST3GAL6 | cDNA Transcript | NM_001323360.1 |
| | | Variant 11 | |
| SEQ ID NO. 742 | ST3GAL6 | Amino Acid | NP_001310289.1 |
| | | Sequence Isoform 2 | |
| SEQ ID NO. 743 | ST3GAL6 | cDNA Transcript | NM_001323362.1 |
| | | Variant 12 | |
| SEQ ID NO. 744 | ST3GAL6 | Amino Acid | NP_001310291.1 |
| | | Sequence isoform 5 | |
| SEQ ID NO. 745 | ST3GAL6 | cDNA Transcript | NM_001323363.1 |
| | | Variant 13 | |
| SEQ ID NO. 746 | ST3GAL6 | Amino Acid | NP_001310292.1 |
| | | Sequence Isoform 5 | |
| SEQ ID NO. 747 | ST3GAL6 | cDNA Transcript | NM_001323364.1 |
| | | Variant 14 | |
| SEQ ID NO. 748 | ST3GAL6 | Amino Acid | NP_001310293.1 |
| | | Sequence Isoform 5 | |
| SEQ ID NO. 749 | ST3GAL6 | cDNA Transcript | NM_001323365.1 |
| | | Variant 8 | |
| SEQ ID NO. 750 | ST3GAL6 | Amino Acid | NP_001310294.1 |
| | | Sequence Isoform 1 | |
| SEQ ID NO. 751 | ST3GAL6 | cDNA Transcript | NM_001323366.1 |
| | | Variant 10 | |
| SEQ ID NO. 752 | ST3GAL6 | Amino Acid | NP_001310295.1 |
| | | Sequence isoform 2 | |
| SEQ ID NO. 753 | ST3GAL6 | cDNA Transcript | NM_001323367.1 |
| | | Variant 16 | |
| SEQ ID NO. 754 | ST3GAL6 | Amino Acid | NP_001310296.1 |
| | | Sequence Isoform 6 | |
| SEQ ID NO. 755 | ST3GAL6 | cDNA Transcript | NM_001323368.1 |
| | | Variant 9 | |
| SEQ ID NO. 756 | ST3GAL6 | Amino Acid | NP_001310297.1 |
| | | Sequence Isoform 1 | |
| SEQ ID NO. 757 | ST3GAL6 | cDNA Transcript | NM_006100.3 |
| | | Variant 1 | |
| SEQ ID NO. 758 | ST3GAL6 | Amino Acid | NP_006091.1 |
| | | Sequence Isoform 1 | |
| SEQ ID NO. 759 | LAMP1 | cDNA | NM_005561.3 |
| SEQ ID NO. 760 | LAMP1 | Amino Acid | NP_005552.3 |
| | | Precursor | |
| SEQ ID NO. 761 | LGALS1 | cDNA | NM_002305.3 |
| SEQ ID NO. 762 | LGALS1 | Amino Acid | NP_002296.1 |
| | | Sequence | |
| SEQ ID NO. 763 | CD34 | Amino Acid | |
| | | Sequence | |
| SEQ ID NO. 764 | CD45RA | Amino Acid | |
| | | Sequence | |
| SEQ ID NO. 765 | CD90 | Amino Acid | |
| | | Sequence | |
| SEQ ID NO. 766 | CD123 | Amino Acid | |
| | | Sequence | |
| SEQ ID NO. 767 | CD38 | Amino Acid | |
| | | Sequence | |
| SEQ ID NO. 768 | CD19 | Amino Acid | |
| | | Sequence | |
| SEQ ID NO. 769 | CD10 | Amino Acid | |
| | | Sequence | |
| SEQ ID NO. 770 | CD33 | Amino Acid | |
| | | Sequence | |
| SEQ ID NO. 771 | CD45 | Amino Acid | |
| | | Sequence | |
| SEQ ID NO. 772 | CD34 | Nucleic Acid | |
| | | Sequence | |
| SEQ ID NO. 773 | CD45RA | Nucleic Acid | |
| | | Sequence | |
| SEQ ID NO. 774 | CD90 | Nucleic Acid | |
| | | Sequence | |
| SEQ ID NO. 775 | CD123 | Nucleic Acid | |
| | | Sequence | |
| SEQ ID NO. 776 | CD38 | Nucleic Acid | |
| | | Sequence | |
| SEQ ID NO. 777 | CD19 | Nucleic Acid | |
| | | Sequence | |
| SEQ ID NO. 778 | CD10 | Nucleic Acid | |
| | | Sequence | |
| SEQ ID NO. 779 | CD33 | Nucleic Acid | |
| | | Sequence | |
| SEQ ID NO. 780 | CD45 | Nucleic Acid | |
| | | Sequence | |

CD34 amino acid sequence
(SEQ ID NO: 763)
mlvrrgarag prmprgwtal cllsllpsgf msldnngtat pelptqgtfs nvstnvsyqe
tttpstlgst slhpvsqhgn eattnitett vkftstsvit svygntnssv qsqtsvistv
fttpanvstp ettdkpslsp gnvsdlstts tslatsptkp ytssspilsd ikaeikcsgi
revkltqgic leqnktssca efkkdrgegl arvlcgeeqa dadagaqvcs lllaqsevrp
qclllvlanr teisskiqlm kkhqsdlkkl gildfteqdv ashqsysqkt lialvtsgal
lavigitgyf lmnrrswspt gerlelep
CD45RA amino acid sequence
(SEQ ID NO: 764)
MYLWLKLLAFGHAFLDTEVFVTGQPTPSPTDAYLNASETTTLS
PSGSAVISTTTIATTPSKPTCDEKYANITVDYLYNKETKLFTAKLNVNENVECGNNTC
TNNEVHNLTECKNASVSISHNSCTAPDKTLILDVPPGVEKFQLHDCTQVEKADTTICL
KWKNIETFTCDTQNITYRFQCGNMIFDNKEIKLENLEPEHEYKCDSEILYNNHKFTNA
SKIIKTDFGSPGEPQIIFCRSEAAHQGVITWNPPQRSFHNFTLCYIKETEKDCLNLDK
NLIKYDLQNLKPYTKYVLSLHAYIIAKVQRNGSAAMCHFTTKSAPPSQVWNMTVSMTS
DNSMHVKCRPPRDRNGPHERYHLEVEAGNTLVRNESHKNCDFRVKDLQYSTDYTFKAY
FHNGDYPGEPFILHHSTSYNSKALIAFLAFLIIVTSIALLVVLYKIYDLHKKRSCNLD
EQQELVERDDEKQLMNVEPIHADILLETYKRKIADEGRLFLAEFQSIPRVFSKFPIKE
ARKPFNQNKNRYVDILPYDYNRVELSEINGDAGSNYINASYIDGFKEPRKYIAAQGPR
DETVDDFWRMIWEQKATVIVMVTRCEEGNRNKCAEYWPSMEEGTRAFGDVVVKINQHK
RCPDYIIQKLNIVNKKEKATGREVTHIQFTSWPDHGVPEDPHLLLKLRRRVNAFSNFF
SGPIVVHCSAGVGRTGTYIGIDAMLEGLEAENKVDVYGYVVKLRRQRCLMVQVEAQYI
LIHQALVEYNQFGETEVNLSELHPYLHNMKKRDPPSEPSPLEAEFQRLPSYRSWRTQH
IGNQEENKSKNRNSNVIPYDYNRVPLKHELEMSKESEHDSDESSDDDSDSEEPSKYIN
ASFIMSYWKPEVMIAAQGPLKETIGDFWQMIFQRKVKVIVMLTELKHGDQEICAQYWG
EGKQTYGDIEVDLKDTDKSSTYTLRVFELPHSKRKDSRTVYQYQYTNWSVEQLPAEPK
ELISMIQVVKQKLPQKNSSEGNKHHKSTPLLIHCRDGSQQTGIFCALLNLLESAETEE
VVDIFQVVKALRKARPGMVSTFEQYQFLYDVIASTYPAQNGQVKKNNHQEDKIEFDNE
VDKVKQDANCVNPLGAPEKLPEAKEQAEGSEPTSGTEGPEHSVNGPASPALNQGS
CD90 amino acid sequence
(SEQ ID NO: 765)
MNLAISIALLLTVLQVSRGQKVTSLTACLVDQSLRLDCRHENTS
SSPIQYEFSLTRETKKHVLFGTVGVPEHTYRSRTNFTSKYNMKVLYLSAFTSKDEGTY
TCALHHSGHSPPISSQNVTVLRDKLVKCEGISLLAQNTSWLLLLLLSLSLLQATDFMS
L
CD123 amino acid sequence
(SEQ ID NO: 766)
MVLLWLTLLLIALPCLLQTKEDPNPPITNLRMKAKAQQLTWDLN
RNVTDIECVKDADYSMPAVNNSYCQFGAISLCEVTNYTVRVANPPFSTWILFPENSGK
PWAGAENLTCWIHDVDFLSCSWAVGPGAPADVQYDLYLNVANRRQQYECLHYKTDAQG
TRIGCRFDDISRLSSGSQSSHILVRGRSAAFGIPCTDKFVVFSQIEILTPPNMTAKCN
KTHSFMHWKMRSHFNRKFRYELQIQKRMQPVITEQVRDRTSFQLLNPGTYTVQIRARE
RVYEFLSAWSTPQRFECDQEEGANTRAWRTSLLIALGTLLALVCVFVICRRYLVMQRL
FPRIPHMKDPIGDSFQNDKLVVWEAGKAGLEECLVTEVQVVQKT
CD38 amino acid sequence
(SEQ ID NO: 767)
MANCEFSPVSGDKPCCRLSRRAQLCLGVSILVLILVVVLAVVVP
RWRQQWSGPGTTKRFPETVLARCVKYTEIHPEMRHVDCQSVWDAFKGAFISKHPCNIT
EEDYQPLMKLGTQTVPCNKILLWSRIKDLAHQFTQVQRDMFTLEDTLLGYLADDLTWC
GEFNTSKINYQSCPDWRKDCSNNPVSVFWKTVSRRFAEAACDVVHVMLNGSRSKIFDK
NSTFGSVEVHNLQPEKVQTLEAWVIHGGREDSRDLCQDPTIKELESIISKRNIQFSCK
NIYRPDKFLQCVKNPEDSSCTSEI
CD19 amino acid sequence
(SEQ ID NO: 768)
MPPPRLLFFLLFLTPMEVRPEEPLVVKVEEGDNAVLQCLKGTSD
GPTQQLTWSRESPLKPFLKLSLGLPGLGIHMRPLAIWLFIFNVSQQMGGFYLCQPGPP
SEKAWQPGWTVNVEGSGELFRWNVSDLGGLGCGLKNRSSEGPSSPSGKLMSPKLYVWA
KDRPEIWEGEPPCLPPRDSLNQSLSQDLTMAPGSTLWLSCGVPPDSVSRGPLSWTHVH
PKGPKSLLSLELKDDRPARDMWVMETGLLLPRATAQDAGKYYCKRGNLTMSFHLEITA
RPVLWHWLLRTGGWKVSAVTLAYLIFCLCSLVGILHLQRALVLRRKRKRMTDPTRRFF
KVTPPPGSGPQNQYGNVLSLPTPTSGLGRAQRWAAGLGGTAPSYGNPSSDVQADGALG
SRSPPGVGPEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSYEDMPGILY
AAPQLRSIRGQPGPNHEEDADSYENMDNPDGPDPAWGGGGRMGTWSTR
CD10 amino acid sequence
(SEQ ID NO: 769)
MGKSESQMDITDINTPKPKKKQRWTPLEISLSVLVLLLTIIAVTMIALYATYDDGICKSS
DCIKSAARLIQNMDATTEPCTDFFSKYACGGWLKRNVIPETSSRYGNFDILRDELEVVLKD
VLQEPKTEDIVAVQKAKALYRSCINESAIDSRGGEPLLKLLPDIYGWPVATENWEQKYGA
SWTAEKAIAQLNSKYGKKVLINLFVGTDDKNSVNHVIHIDQPRLGLPSRDYYSCTGIYKE
ACTAYVDFMISVARLIRQEERLPIDENQLALEMNKVMELEKEIANATAKPEDRNDPMLLY
NKMTLAQIQNNFSLEINGKPFSWLNFTNEIMSTVNISITNEEDVVVYAPEYLTKLKPILT
KYSARDLQNLMSWRFIMDLVSSLSRTYKESRNAFRKALYGTTSETATWRRCANYVNGNME
NAVGRLYVEAAFAGESKHVVEDLIAQIREVFIQTLDDLTWMDAETKKRAEEKALAIKERI
GYPDDIVSNDNKLNNEYLELNYKEDEYFENIIQNLKFSQSKQLKKLREKVDKDEWISGAA
VVNAFYSSGRNQIVFPAGILQPPFFSAQQSNSLNYGGIGMVIGHEITHGFDDNGRNFNKD
GDLVDWWTQQSASNFKEQSQCMVYQYGNFSWDLAGGQHLNGINTLGENIADNGGLGQAYR
AYQNYIKKNGEEKLLPGLDLNHKQLFFLNFAQVWCGTYRPEYAVNSIKTDVHSPGNFRII
GTLQNSAEFSEAFHCRKNSYMNPEKKCRVW
CD33 amino acid sequence
(SEQ ID NO: 770)
MPLLLLLPLLWAGALAMDPNFWLQVQESVTVQEGLCVLVPCTFFHPIPYYDKNSPVHGYW
FREGAIISRDSPVATNKLDQEVQEETQGRFRLLGDPSRNNCSLSIVDARRRDNGSYFFRM
ERGSTKYSYKSPQLSVHVTDLTHRPKILIPGTLEPGHSKNLTCSVSWACEQGTPPIFSWL
SAAPTSLGPRTTHSSVLIITPRPQDHGTNLTCQVKFAGAGVTTERTIQLNVTYVPQNPTT
GIFPGDGSGKQSTRAGVVHGAIGGAGVTALLALCLCLIFFIVKTHRRKAARTAVGRNDTH
PTTGSASPKHQKKSKLHGPTETSSCSGAAPTVEMDEELHYASLNFHGMNPSKDTSTEYSE
VRTQ
CD45 amino acid sequence
(SEQ ID NO: 771)
MYLWLKLLAFGFAFLDTEVFVTGQSPTPSPTGLTTAKMPSVPLSSDPLPTHTTAFSPAST
FERENDFSETTTSLSPDNTSTQVSPDSLDNASAFKTTGVSSVQTPHLPTHADSQTPSAGT
DTQTFSGSAANAKLNPTPGSNAISDVPGSRSTASTFPTDPVSPLTTTLSLAKHSSAALPA
RTSNTTITANTSDAYLNASETTTLSPSGSAVISTTTIATTPSKPTCDEKYANITVDYLYN
KETKLFTAKLNVNENVECGNNTCTNNEVHNLTECKNASVSISHNSCTAPDKTLILDVPPG
VEKFQLHDCTQVEKADTTICLKWKNIETFTCDTQNITYRFQCGNMIFDNKEIKLENLEPE
HEYKCDSEILYNNHKFTNASKIIKTDFGSPGEPQIIFCRSEAAHQGVITWNPPQRSFHMF
TLCYIKETEKDCLNLDKNLIKYDLQNLKPYTKYVLSLHAYIIAKVQRNGSAAMCHFTTKS
APPSQVWNMTVSMTSDNSMHVKCRPPRDRNGPHERYHLEVEAGNTLVRNESHKNCDFRVK
DLQYSTDYTFKAYFHNGDYPGEPFILHHSTSYNSKALIAFLAFLIIVTSIALLVVLYKIY
DLHKKRSCNLDEQQELVERDDEKQLMNVEPIHADILLETYKRKIADEGRLFLAEFQSIPR
VFSKFPIKEARKPFNQNKNRYVDILPYDYNRVELSEINGDAGSNYINASYIDGFKEPRKY
IAAQGPRDETVDDFWRMIWEQKATVIVMVTRCEEGNRNKCAEYWPSMEEGTRAFGDVVVK
INQHKRCPDYIIQKLNIVNKKEKATGPEVTHIQFTSWPDHGVPEDPHLLLKLRRRVNAFS
NFFSGPIVVHCSAGVGRTGTYIGIDAMLEGLEAENKVDVYGYVVKLRRQRCLMVQVEAQY
ILIHQALVEYNQFGETEVNLSELHPYLHNMKKRDPPSEPSPLEAEFQRLPSYRSWRTQHI
GNQEENKSKNRNSNVIPYDYNRVPLKHELEMSKESEHDSDESSDDDSDSEEPSKYINASF
IMSYWKPEVMIAAQGPLKETIGDFWQMIFQRKVKVIVMLTELKHGDQEICAQYWGEGKQT
YGDIEVDLKDTDKSSTYTLRVFELRHSKRKDSRTVYQYQYTNWSVEQLPAEPKELISMIQ
VVKQKLPQKNSSEGNKHHKSTPLLIHCRDGSQQTGIFCALLNLLESAETEEVVDIFQVVK
ALRKARPGMVSTFEQYQFLYDVIASTYPAQNGQVKKNNHQEDKIEFDNEVDKVKQDANCV
NPLGAPEKLPFEAKEQAEGSEPTSGTEGPEHSVNGPASPALNQGS
CD34 nucleic acid sequence
(SEQ ID NO: 772)
atgctggtcc gcaggggcgc gcgcgcaggg cccaggatgc cgcggggctg gaccgcgctt
tgcttgctga gtttgctgcc ttctgggttc atgagtcttg acaacaacgg tactgctacc
ccagagttac ctacccaggg aacattttca aatgtttcta caaatgtatc ctaccaagaa
actacaacac ctagtaccct tggaagtacc agcctgcacc ctgtgtctca acatggcaat
gaggccacaa caaacatcac agaaacgaca gtcaaattca catctacctc tgtgataacc
tcagtttatg gaaacacaaa ctcttctgtc cagtcacaga cctctgtaat cagcacagtg
ttcaccaccc cagccaacgt ttcaactcca gagacaacct tgaagcctag cctgtcacct
ggaaatgttt cagacctttc aaccactagc actagccttg caacatctcc cactaaaccc
tatacatcat cttctcctat cctaagtgac atcaaggcag aaatcaaatg ttcaggcatc
agagaagtga aattgactca gggcatctgc ctggagcaaa ataagacctc cagctgtgcg
gagtttaaga aggacagggg agagggcctg gcccgagtgc tgtgtgggga ggagcaggct
gatgctgatg ctggggccca ggtatgctcc ctgctccttg cccagtctga ggtgaggcct
cagtgtctac tgctggtctt ggccaacaga acagaaattt ccagcaaact ccaacttatg
aaaaagcacc aatctgacct gaaaaagctg gggatcctag atttcactga gcaagatgtt
gcaagccacc agagctattc ccaaaagacc ctgattgcac tggtcacctc gggagccctg
ctggctgtct tgggcatcac tggctatttc ctgatgaatc gccgcagctg gagocccaca
ggagaaaggc tgggcgaaga cccttattac acggaaaacg gtggaggcca gggctatagc
tcaggacctg ggacctcccc tgaggctcag ggaaaggcca gtgtgaaccg aggggctcag
gaaaacggga ccggccaggc cacctccaga aacggccatt cagcaagaca acacgtggtg
gctgataccg aattgtga
CD45RA nucleic acid seguence
(SEQ ID NO: 773)
atgtatttgt ggcttaaact cttggcattt ggctttgcct ttctggacac agaagtattt
gtgacagggc aaagoccaac accttccccc actggattga ctacagcaaa gatgcccagt
gttccacttt caagtgaccc cttacctact cacaccactg cattctcacc cgcaagcacc
tttgaaagag aaaatgactt ctcagagacc acaacttctc ttagtccaga caatacttcc
acccaagtat ccccggactc tttggataat gctagtgctt ttaataccac aggtgtttca
tcagtacaga cgcctcacct tcccacgcac gcagactcgc agacgccctc tgctggaact
gacacgcaga cattcagcgg ctccgccgcc aatgcaaaac tcaaccctac cccaggcagc
aatgctatct cagatgtccc aggagagagg agtacagcca gcacctttcc tacagaccca
gtttccccat tgacaaccac cctcagcctt gcacaccaca gctctgctgc cttacctgca
cgcacctcca acaccaccat cacagcgaac acctcagatg cctaccttaa tgcctctgaa
acaaccactc tgagcccttc tggaagcgct gtcatttcaa ccacaacaat agctactact
ccatctaagc caacatgtga tgaaaaatat gcaaacatca ctgtggatta cttatataac
aaggaaacta aattatttac agcaaagcta aatgttaatg agaatgtgga atgtggaaac
aatacttgca caaacaatga ggtgcataac cttacagaat gtaaaaatgc gtctgtttcc
atatctcata attcatgtac tgctcctgat aagacattaa tattagatgt gccaccaggg
gttgaaaagt ttcagttaca tgattgtaca caagttgaaa aagcagatac tactatttgt
ttaaaatgga aaaatattga aacctttact tgtgatacac agaatattac ctacagattt
cagtgtggta atatgatatt tgataataaa gaaattaaat tagaaaacct tgaacccgaa
catgagtata agtgtgactc agaaatactc tataataacc acaagtttac taacgcaagt
aaaattatta aaacagattt tgggagtcca ggagagcctc agattatttt ttgtagaagt
gaagctgcac atcaaggagt aattacctgg aatccccctc aaagatcatt tcataatttt
accctctgtt atataaaaga gacagaaaaa gattgcctca atctggataa aaacctgatc
aaatatgatt tgcaaaattt aaaaccttat acgaaatatg ttttatcatt acatgcctac
atcattgcaa aagtgcaacg taatggaagt gctgcaatgt gtcatttcac aactaaaagt
gctcctccaa gccaggtctg gaacatgact gtctccatga catcagataa tagtatgcat
gtcaagtgta ggcctcccag ggaccgtaat ggcccccatg aacgttacca tttggaagtt
gaagctggaa atactctggt tagaaatgag tcgcataaga attgcgattt ccgtgtaaaa
gatcttcaat attcaacaga ctacactttt aaggcctatt ttcacaatgg agactatcct
ggagaaccct ttattttaca tcattcaaca tcttataatt ctaaggcact gatagcattt
ctggcatttc tgattattgt gacatcaata gccctgcttg ttgttctcta caaaatctat
gatctacata agaaaagatc ctgcaattta gatgaacagc aggagcttgt tgaaagggat
gatgaaaaac aactgatgaa tgtggagcca atccatgcag atattttgtt ggaaacttat
aagaggaaga ttgctgatga aggaagactt tttctggctg aatttcagag catcccgcgg
gtgttcagca agtttcctat aaaggaagct cgaaagccct ttaaccagaa taaaaaccgt
tatgttgaca ttcttcctta tgattataac cgtgttgaac tctctgagat aaacggagat
gcagggtcaa actacataaa tgccagctat attgatggtt tcaaagaacc caggaaatac
attgctgcac aaggLoccag ggatgaaact gttgatgatt tctgmaggat gatttgggaa
cagaaagcca cagttattgt catggtcact cgatgtgaag aaggaaacag gaacaagtgt
gcagaatact ggccgtcaat ggaagagggc actcgggctt ttggagatgt tgttgtaaag
atcaaccagc acaaaagatg tccagattac atcattcaga aattgaacat tgtaaataaa
aaagaaaaag caactggaag agaggtgact cacattcagt tcaccagctg gccagaccac
ggggtgcctg aggatcctca cttgctcctc aaactgagaa ggagagtgaa tgccttcagc
aatttcttca gtggtcccat tgtggtgcac tgcagtgctg gtgttgggcg cacaggaacc
tatatcggaa ttgatgccat gctagaaggc ctggaagccg agaacaaagt ggatgtttat
ggttatgttg tcaagctaag gcgacagaga tgcctgatgg ttcaagtaga ggcccagtac
atcttgatcc atcaggcttt ggtggaatac aatcagtttg gagaaacaga agtgaatttg
tctgaattac atccatatct acataacatg aagaaaaggg atccacccag tgagccgtct
ccactagagg ctgaattcca gagacttcct tcatatagga gctggaggac acagcacatt
ggaaatcaag aagaaaataa aagtaaaaac aggaattcta atgtcatccc atatgactat
aacagagtgc cacttaaaca tgagctggaa atgagtaaag agagtgagca tgattcagat
gaatcctctg atgatgacag tgattcagag gaaccaagca aatacatcaa tgcatctttt
ataatgagct actggaaacc tgaagtgatg attgctgctc agggaccact gaaggagacc
attggtgact tttggcagat gatcttccaa agaaaagtca aagttattgt tatgctgaca
gaactgaaac atggagacca ggaaatctgt gctcagtact ggggagaagg aaagcaaaca
tatggagata ttgaagttga cctgaaagac acagacaaat cttcaactta tacccttcgt
gtctttgaac tgagacattc caagaggaaa gactctcgaa ctgtgtacca gtaccaatat
acaaactgga gtgtggagca gcttcctgca gaacccaagg aattaatctc tatgattcag
gtcgtcaaac aaaaacttcc ccagaagaat tcctctgaag ggaacaagca tcacaagagt
acacctctac tcattcactg cagggatgga tatcagcaaa cgggaatatt ttgtgctttg
ttaaatctct tagaaagtgc ggaaacagaa gaggtagtgg atatttttca agtggtaaaa
gctctacgca aagctaggcc aggcatggtt tccacattcg agcaatatca attcctatat
gacgtcattg ccagcaccta ccctgctcag aatggacaag taaagaaaaa caaccatcaa
gaagataaaa ttgaatttga taatgaagtg gacaaagtaa agcaggatgc taattgtgtt
aatccacttg gtgccccaga aaagctccct gaagcaaagg aacaggctga aggttctgaa
cccacgagtg gcactgaggg gccagaacat tctgtcaatg gtcctgcaag tccagcttta
aatcaaggtt catag
CD90 nucleic acid sequence
(SEQ ID NO: 774)
atgaacctgg ccatcagcat cgctctoctg ctaacagtct tgcaggtctc ccgagggcag
aaggtgacca gcctaacggc ctgcctagtg gaccagagcc ttcgtctgga ctgccgccat
gagaatacca gcagttcacc catccagtac gagttcagcc tgacccgtga gacaaagaag
cacgtgctct ttggcactgt gggggtgcct gagcacacat accgctcccg aaccaacttc
accagcaaat acaacatgaa ggtcctctac ttatccgcct tcactagcaa ggacgagggc
acctacacgt gtgcactcca ccactctggc cattccccac ccatctcctc ccagaacgtc
acagtgctca gagacaaact ggtcaagtgt gagggcatca gcctgctggc tcagaacacc
tcgtggctgc tgctgctcct gctctccctc tccctcctcc aggccacgga tttcatgtcc
ctgtga
CD123 nucleic acid sequence
(SEQ ID NO: 775)
atggtcctcc tttggctcac gctgctectg atcgccctgc cctgtotcct gcaaacgaag
gaagatccaa acccaccaat cacgaaccta aggatgaaag caaaggctca gcagttgacc
tgggacctta acagaaatgt gaccgatatc gagtgtgtta aagacgccga ctattctatg
ccggcagtga acaatagcta ttgccagttt ggagcaattt ccttatgtga agtgaccaac
tacaccgtcc gagtggccaa cccaccattc tccacgtgga tcctcttccc tgagaacagt
gggaagcctt gggcaggtgc ggagaatctg acctgctgga ttcatgacgt ggatttcttg
agctgcagct gggcggtagg cccgggggcc cccgcggacg tccagtacga cctgtacttg
aacgttgcca acaggcgtca acagtacgag tgtcttcact acaaaacgga tgctcaggga
acacgtatcg ggtgtcgttt cgatgacatc tctcgactct ccagcggttc tcaaagttcc
cacatcctgg tgcggggcag gagcgcagcc ttcggtatcc cctgcacaga taagtttgtc
gtcttttcac agattgagat attaactcca cccaacatga ctgcaaagtg taataagaca
cattccttta tgcactggaa aatgagaagt catttcaatc gcaaatttcg ctatgagctt
cagatacaaa agagaatgca gcctgtaatc acagaacagg tcagagacag aacctccttc
cagctactca atcctggaac gtacacagta caaataagag cccgggaaag agtgtatgaa
ttcttgagcg cctggagcac cccccagcgc ttcgagtgcg accaggagga gggcgcaaac
acacgtgcct ggcggacgtc gctgctgatc gcgctgggga cgctgctggc cctggtctgt
gtcttcgtga tctgcagaag gtatctggtg atgcagagac tctttccccg catccctcac
atgaaagacc ccatcggtga cagottccaa aacgacaagc tggtggtctg ggaggcgggc
aaagccggcc tggaggagtg tctggtgact gaagtacagg tcgtgcagaa aacttga
CD38 nucleic acid sequence
(SEQ ID NO: 776)
atggccaact gcgagttcag cccggtgtcc ggggacaaac cctgctgccg gctctctagg
agagcccaac tctgtcttgg cgtcagtatc ctggtcctga tcctcgtcgt ggtgctcgcg
gtggtcgtcc cgaggtggcg ccagcagtgg agoggtccgg gcaccaccaa gcgctttccc
gagaccgtcc tggcgcgatg cgtcaagtac actgaaattc atcctgagat gagacatgta
gactgccaaa gtgtatggga tgctttcaag ggtgcattta tttcaaaaca tccttgcaac
attactgaag aagactatca gccactaatg aagttgggaa ctcagaccgt accttgcaac
aagattcttc tttggagcag aataaaagat ctggcccatc agttcacaca ggtccagcgg
gacatgttca ccctggagga cacgctgcta ggctaccttg ctgatgacct cacatggtgt
ggtgaattca acacttccaa aataaactat caatcttgcc cagactggag aaaggactgc
agcaacaacc ctgtttcagt attctggaaa acggtttccc gcaggtttgc agaagctgcc
tgtgatgtgg tccatgtgat gctcaatgga tcccgcagta aaatctttga caaaaacagc
acttttggga gtgtgmaagt ccataatttg caaccagaga aggbtcagac actagaggcc
tgggtgatac atggtggaag agaagattcc agagacttat gccaggatcc caccataaaa
gagctggaat cgattataag caaaaggaat attcaatttt cctgcaagaa tatctacaga
cctgacaagt ttcttcagtg tgtgaaaaat cctgaggatt catcttgcac atctgagatc
tga
CD19 nucleic acid sequence
(SEQ ID NO: 777)
atgccacctc ctcgcctcct cttcttcctc ctcttcctca cccccatgga agtcaggccc
gaggaacctc tagtggtgaa ggtggaagag ggagataacg ctgtgctgca gtgcctcaag
gggacctcag atggccccac tcagcagctg acctggtctc gggagtcccc gcttaaaccc
ttcttaaaac tcagcctggg gctgccaggc ctgggaatcc acatgaggcc cctggccatc
tggcttttca tcttcaacgt ctctcaacag atggggggct tctacctgtg ccagccgccn
cccccctctg agaaggcctg gcagcctggc tggacagtca atgtggaggg cagcggggag
ctgttccggt ggaatgtttc ggacctaggt ggcctgggct gtggcctgaa gaacaggtcc
tcagagggcc ccagctcccc ttccgggaag ctcatgagcc ccaagctgta tgtgtgggcc
aaagaccgcc ctgagatctg ggagggagag cctccgtgtc tcccaccgag ggacagcctg
aaccagagcc tcagccagga cctcaccatg gcccctggct ccacactctg gctgtcctgt
ggggtacccc ctgactctgt gtccaggggc cccctctcct ggacccatgt gcaccccaag
gggcctaagt cattgctgag cctagagctg aaggacgatc gcccggccag agatatgtgg
gtaatggaga cgggtctgtt gttgccccgg gccacagctc aagacgctgg aaagtattat
tgtcaccgtg gcaacctgac catgtcattc cacctggaga tcactgctcg gccagtacta
tggcactggc tgctgaggac tggtggctgg aaggtctcag ctgtgacttt ggcttatctg
atcttctgcc tgtgttccct tgtgggcatt cttcatcttc aaagagccct ggtcctgagg
aggaaaagaa agcgaatgac tgaccccacc aggagattct tcaaagtgac gcctccccca
ggaagcgggc cccagaacca gtacgggaac gtgctgtctc tccccacacc cacctcaggc
ctcggacgcg cccagcgttg ggccgcaggc ctggggggca ctgccccgtc ttatggaaac
ccgagcagcg acgtccaggc ggatggagcc ttggggtccc ggagcccgcc gggagtgggc
ccagaagaag aggaagggga gggctatgag gaacctgaca gtgaggagga ctccgagttc
tatgagaacg actccaacct tgggcaggac cagctctccc aggatggcag cggctacgag
aaccctgagg atgagcccct gggtcctgag gatgaagact ccttctccaa cgctgagtct
tatgagaacg aggatgaaga gctgacccag ccggtcgcca ggacaatgga cttcctgagc
cctcatgggt cagcctggga ccccagccgg gaagcaacct ccctggggtc ccagtcctat
gaggatatga gaggaatcct gtatgcagcc ccccagctcc gctccattcg gggccagcct
ggacccaatc atgaggaaga tgcagactct tatgagaaca tggataatcc cgatgggcca
gacccagcct ggggaggagg gggccgcatg ggcacctgga gcaccaggtg a
CD10 nucleic acid sequence
(SEQ ID NO: 778)
ATGGGCAAGTCAGAAAGTCAGATGGATATAACTGATATCAACACTCCAAAGCCAAAGAAGAAACAGCGAT
GGACTCCACTGGAGATCAGCCTCTCGGTCCTTGTCCTGCTCCTCACCATCATAGCTGTGACAATGATCGC
ACTCTATGCAACCTACGATGATGGTATTTGCAAGTCATCAGACTGCATAAAATCAGCTGCTCGACTGATC
CAAAACATGGATGCCACCACTGAGCCTTGTACAGACTTTTTCAAATATGCTTGCGGAGGCTGGTTGAAAC
GTAATGTCATTCCCGAGACCAGCTCCCGTTACGGCAACTTTGACATTTTAAGAGATGAACTAGAAGTCGT
TTTGAAAGATGTCCTTCAAGAACCCAAAACTGAAGATATAGTAGCAGTGCAGAAAGCAAAAGCATTGTAC
AGGTCTTGTATAAATGAATCTGCTATTGATAGCAGAGGTGGAGAACCTGTACTCAAACTGTTAGCAGACA
TATATGGGTGGCCAGTAGCAACAGAAAACTGGGAGCAAAAATATGGTGCTTCTTGGACAGCTGAAAAAGC
TATTGCACAACTGAATTCTAAATATGGGAAAAAAGTCCTTATTAATTTGTTTGTTGGCACTGATGATAAG
AATTCTGTGAATCATGTAATTGATATTGACCAACGTCGACTTGGCCTCGCTTCTAGAGATTACTATGAAT
GCACTGGAATCTATAAAGAGGCTTGTACAGCATATGTGGATTTTATGATTTCTGTGGCCAGATTGATTCG
TCAGGAAGAAAGATTGCCCATCGATGAAAACCAGCTTGCTTTGGAAATGAATAAAGTTATGGAATTGGAA
AAAGAAATTGCCAATGCTACGGCTAAACCTGAAGATCGAAATGATCCAATGCTTCTGTATAACAAGATGA
CATTGGCCCAGATCCAAAATAACTTTTCACTAGAGATCAATGGGAAGCCATTCAGCTGGTTGAATTTCAC
AAATGAAATCATGTCAACTGTGAATATTAGTATTACAAATGAGGAAGATGTGGTTGTTTATGCTCCAGAA
TATTTAACCAAACTTAAGCCCATTCTTACCAAATATTCTGCCAGAGATCTTCAAAATTTAATGTCCTGGA
GATTCATAATGGATCTTGTTAGCAGCCTCAGCCGAACCTACAAGGAGTCCAGAAATGCTTTCCGCAAGGC
CCTTTATGGTACAACCTCAGAAACAGCAACTTGGAGACGTTGTGCAAACTATGTCAATGGGAATATGGAA
AATGCTGTGGGGAGGCTTTATGTGGAAGCAGCATTTGCTGGAGAGAGTAAACATGTGGTCGAGGATTTGA
TTGCACAGATCCGAGAAGTTTTTATTCAGACTTTAGATGACCTCACTTGGATGGATGCCGAGACAAAAAA
GAGAGCTGAAGAAAAGGCCTTAGGAATTAAAGAAAGGATGGGCTATGCTGATGACATTGTTTCAAATGAT
AACAAACTGAATAATGAGTACCTCGAGTTGAACTACAAAGAAGATGAATACTTCGAGAACATAATTCAAA
ATTTGAAATTCAGCCAAAGTAAACAACTGAAGAAGCTCCGAGAAAAGGTGGACAAAGATGAGTGGATAAG
TGGAGCAGCTGTAGTCAATGCATTTTACTCTTCAGGAAGAAATCAGATAGTCTTCCCAGCCGGCATTCTG
CAGCCCCCCTTCTTTAGTGCCCAGCAGTCCAACTCATTGAACTATGGGGGCATCGGCATGGTCATAGGAC
ACGAAATCACCCATGGCTTCGATGACAATGGCAGAAACTTTAACAAAGATGGAGACCTCGTTGACTGGTG
GACTCAACAGTCTGCAAGTAACTTTAAGGAGCAATCCCAGTGCATGGTGTATCAGTATGGAAACTTTTCC
TGGGACCTGGCAGGTGGACAGCACCTTAATGGAATTAATACACTGGGAGAAAACATTGCTGATAATGGAG
GTCTTGGTCAAGCATACAGAGCCTATCAGAATTATATTAAAAAGAATGGCGAAGAAAAATTACTTCCTGG
ACTTGACCTAAATCACAAACAACTATTTTTCTTGAACTTTGCACAGGTGTGGTGTGGAACCTATAGGCCA
GAGTATGCGGTTAACTCCATTAAAACAGATGTGCACAGTCCAGGCAATTTCAGGATTATTGGGACTTTGC
AGAACTCTGCAGAGTTTTCAGAAGCCTTTCACTGCCGCAAGAATTCATACATGAATCCAGAAAAGAAGTG
CCGGGTTTGGTGA
CD33 nucleic acid sequence
(SEQ ID NO: 779)
ATGCCGCTGCTGCTACTGCTGCCCCTGCTGTGGGCAGGGGCCCTGGCTATGGATCGAAATTTCTGGCTGC
AAGTGCAGGAGTCAGTGACGGTACAGGAGGGTTTGTGCGTCCTCGTGCCCTGCACTTTCTTCCATCCCAT
ACCCTACTACGACAAGAACTCCCCAGTTCATGGTTACTGGTTCCGGGAAGGAGCCATTATATCCAGGGAC
TGTCCAGTGGCCACAAACAAGCTAGATCAAGAAGTACAGGAGGAGACTCAGGGCAGATTCCGCCTCCTTG
GGGATCCCAGTAGGAACAACTGCTCCCTGAGCATCGTAGACGCCAGGAGGAGGGATAATGGTTCATACTT
CTTTCGGATGGAGAGAGGAAGTACCAAATACAGTTACAAATCTCCCCAGCTCTCTGTGCATGTGACAGAC
TTGACCCACAGGCCGAAAATCGTCATCCCTGGCAGTCTAGAACCCGGCCAGTGCAAAAACCTGACGTGCT
CTGTGTCCTGGGCCTGTGAGCAGGGAACACCCCCGATCTTCTCCTGGTTGTCAGCTGCCCCCACCTCCCT
GGGCCCCAGGACTACTCACTCCTCGGTGCTCATAATCACCCCACGGCCCCAGGACCACGGCACCAACCTG
ACCTGTCAGGTGAAGTTCGCTGGAGCTGGTGTGACTACGGAGAGAACCATCCAGCTCAACGTCACCTATG
TTCCACAGAACCCAACAACTGGTATCTTTCCAGGAGATGGCTCAGGGAAACAAGAGACCAGAGCAGGAGT
GGTTCATGGGGCCATTGGAGGAGCTGGTGTTACAGCCCTGCTCGCTCTTTGTCTCTGCCTCATCTTCTTC
ATAGTGAAGACCCACAGGAGGAAAGCAGCCAGGACAGCAGTGGGCAGGAATGACACCCACCCTACCACAG
GGTCAGCCTCCCCGAAACACCAGAAGAAGTCCAAGTTACATGGCCCCACTGAAACCTCAAGCTGTTCAGG
TGCCGCCCCTACTGTGGAGATGGATGAGGAGCTGCATTATGCTTCCCTCAACTTTCATGGGATGAATCCT
TGCAAGGACACCTCCACCGAATACTCAGAGGTCAGGAGCCAGTGA
CD45 nucleic acid sequence
(SEQ ID NO: 780)
ATGACCRTGTATTTGTGGCTTAAACTCTTGGCATTTGGCTTTGCCTTTCTGGACACAGAAGTATTTGTGA
CAGGGCAAAGCCCAACACCTTCCCCCACTGGATTGACTACAGCAAAGATGCCCAGTGTTCCACTTTCAAG
TGACCCCTTACCTACTCACACCACTGCATTCTCACCCGCAAGCACCTTTGAAAGAGAAAATGACTTCTCA
GAGACCACAACTTCTCTTAGTCCAGACAATACTTCCACCCAAGTATCCCCGGACTCTTTGGATAATGCTA
GTGCTTTTAATACCACAGGTGTTTCATCAGTACAGACGCCTCACCTTCGCACGCACGCAGACTCGCAGAC
GCCCTCTGCTGGAACTGACACGCAGACATTCAGCGGCTCCGCCGCCAATGCAAAACTCAACCCTACCCCA
GGCAGCAATGCTATCTCAGATGTCCCAGGAGAGAGGAGTACAGCCAGCACCTTTCCTACAGACCCAGTTT
CCCCATTGACAACCACCCTCAGGCTTGGACACCACAGCTCTGCTGCCTTACCTGGACGCACCTCCAACAC
CACCATCACAGCGAACACCTCAGATGCCTACCTTAATGCCTCTGAAACAACCACTCTGAGCCCTTCTGGA
AGCGCTGTCATTTCAACCACAACAATAGCTACTACTCCATCTAAGCCAACATGTGATGAAAAATATGCAA
ACATCACTGTGGATTACTTATATAACAAGGAAACTAAATTATTTACAGCAAAGCTAAATGTTAATGAGAA
TGTGGAATGTGGAAACAATACTTGCACAAACAATGAGGTGCATAACCTTACAGAATGTAAAAATGGGTCT
GTTTCCATATCTCATAATTCATGTACTGCTCCTGATAAGACATTAATATTAGATGTGCCACCAGGGGTTG
AAAAGTTTCAGTTACATGATTGTACACAAGTTGAAAAAGCAGATACTACTATTTGTTTAAAATGGAAAAA
TATTGAAACCTTTACTTGTGATACACAGAATATTACCTACAGATTTCAGTGTGGTAATATGATATTTGAT
AATAAAGAAATTAAATTAGAAAACCTTGAACCCGAACATGAGTATAAGTGTGACTCAGAAATACTCTATA
ATAACCACAAGTTTACTAACGCAAGTAAAATTATTAAAACAGATTTTGGGAGTCCAGGAGAGCCTCAGAT
TATTTTTTGTAGAAGTGAAGCTGCACATCAAGGAGTAATTACCTGGAATCCCCCTCAAAGATCATTTCAT
AATTTTACCCTCTGTTATATAAAAGAGACAGAAAAAGATTGCCTCAATCTGGATAAAAACCTGATCAAAT
ATGATTTGCAAAATTTAAAACCTTATACGAAATATGTTTTATCATTACATGCCTACATCATTGCAAAAGT
GCAACGTAATGGAAGTGCTGCAATGTGTCATTTCACAACTAAAAGTGCTCCTCCAAGCCAGGTCTGGAAC
ATGACTGTCTCCATGACATCAGATAATAGTATGCATGTCAAGTGTAGGCCTCCCAGGGACCGTAATGGCC
CCCATGAACGTTACCATTTGGAAGTTGAAGCTGGAAATACTCTGGTTAGAAATGAGTCGCATAAGAATTG
CGATTTCCGTGTAAAAGATCTTCAATATTCAACAGACTACACTTTTAAGGCCTATTTTCACAATGGAGAC
TATCCTGGAGAACCCTTTATTTTACATCATTCAACATCTTATAATTCTAAGGCACTGATAGCATTTCTGG
CATTTCTGATTATTGTGACATCAATAGCCCTGCTTGTTGTTCTCTACAAAATCTATGATCTACATAAGAA
AAGATCCTGCAATTTAGATGAACAGCAGGAGCTTGTTGAAAGGGATGATGAAAAACAACTGATGAATGTG
GAGCCAATCCATGCAGATATTTTGTTGGAAACTTATAAGAGGAAGATTGCTGATGAAGGAAGACTTTTTC
TGGCTGAATTTCAGAGCATCCCGCGGGTGTTCAGCAAGTTTCCTATAAAGGAAGCTCGAAAGCCCTTTAA
CCAGAATAAAARCCGTTATGTTGACATTCTTCCTTATGATTATAACCGTGTTGAACTCTCTGAGATAAAC
GGAGATGCAGGGTCAAACTACATAAATGCCAGCTATATTGATGGTTTGAAAGAACCCAGGAAATACATTG
CTGCACAAGGTCCCAGGGATGAAACTGTTGATGATTTCTGGAGGATGATTTGGGAACAGAAAGCCACAGT
TATTGTCATGGTCACTCGATGTGAAGAAGGAAACAGGAACAAGTGTGCAGAATACTGGCCGTCAATGGAA
GAGGGCACTCGGGCTTTTGGAGATGTTGTTGTAAAGATCAACCAGCACAAAAGATGTCCAGATTACATCA
TTCAGAAATTGAACATTGTAAATAAAAAAGAAAAAGCAACTGGAAGAGAGGTGACTCACATTCAGTTCAC
CAGCTGGCCAGACCACGGGGTGCCTGAGGATCCTCACTTGCTCCTCAAACTGAGAAGGAGAGTGAATGCC
TTCAGCAATTTCTTCAGTGGTCCCATTGTGGTGCACTGCAGTGCTGGTGTTGGGCGCACAGGAACCTATA
TCGGAATTGATGCCATGCTAGAAGGCCTGGAAGCCGAGAACAAAGTGGATGTTTATGGTTATGTTGTCAA
GCTAAGGCGACAGAGATGCCTGATGGTTCAAGTAGAGGCCCAGTACATCTTGATCCATCAGGCTTTGGTG
GAATACAATCAGTTTGGAGAAAGAGAAGTGAATTTGTCTGAATTACATCCATATCTACATAACATGAAGA
AAAGGGATCCACCCAGTGAGCCGTCTCCACTAGAGGCTGAATTCCAGAGACTTCCTTCATATAGGAGCTG
GAGGACACAGCACATTGGAAATCAAGAAGAAAATAAAAGTAAAAACAGGAATTCTAATGTCATCCCATAT
GACTATAACAGAGTGCCACTTAAACATGAGCTGGAAATGAGTAAAGAGAGTGAGCATGATTCAGATGAAT
CCTCTGATGATGACAGTGATTCAGAGGAACCAAGCAAATACATCAATGCATCTTTTATAATGAGCTACTG
GAAACCTGAAGTGATGATTGCTGCTCAGGGACCACTGAAGGAGACCATTGGTGACTTTTGGCAGATGATC
TTCCAAAGAAAAGTCAAAGTTATTGTTATGCTGACAGAACTGAAACATGGAGACCAGGAAATCTGTGCTC
AGTACTGGGGAGAAGGAAAGCAAACATATGGAGATATTGAAGTTGACCTGAAAGACACAGACAAATCTTC
AACTTATACCCTTCGTGTGTTTGAACTGAGACATTGGAAGAGGAAAGAGTCTCGAACTGTGTACCAGTAC
CAATATACAAACTGGAGTGTGGAGCAGCTTCCTGCAGAACGCAAGGAATTAATCTGTATGATTCAGGTCG
TCAAACAAAAACTTCCCCAGAAGAATTCCTCTGAAGGGAACAAGGATCACAAGAGTACACGTCTACTCAT
TCACTGCAGGGATGGATCTCAGCAAACGGGAATATTTTGTGCTTTGTTAAATGTCTTAGAAAGTGCGGAA
ACAGAAGAGGTAGTGGATATTTTTCAAGTGGTAAAAGCTCTACGCAAAGCTAGGCCAGGCATGGTTTCCA
CATTCGAGCAATATCAATTCCTATATGACGTCATTGCCAGCACCTACGCTGCTCAGAATGGACAAGTAAA
GAAAAACAACCATCAAGAAGATAAAATTGAATTTGATAATGAAGTGGACAAAGTAAAGCAGGATGCTAAT
TGTGTTAATCCACTTGGTGCCCCAGAAAAGCTCCCTGAAGCAAAGGAACAGGCTGAAGGTTCTGAACCCA
CGAGTGGCACTGAGGGGCCAGAACATTCTGTCAATGGTCCTGCAAGTCCAGCTTTAAATCAAGGTTCATA
G

### EXAMPLES

### Example 1

### Materials & Methods

### Patient Samples

Informed consent was obtained in accordance with the Declaration of Helsinki and with approval from UK Ethics Committees (Oxford 06\Q1606\110; Greater Glasgow and Clyde 10/S0704/2). Mononuclear cells (MNC) were isolated by Histopaque density gradient within 24-48 hours of collection. CD34+ cells were purified using the CD34 Microbead Kit/MACS separation columns (Miltenyi Biotec).

### NSG Xenograft Assay

Experiments were performed in accordance with UK Government Home Office approved Project License 30/2465. 8-14 week old female NSG mice were irradiated 100-125 cGy twice, 4 hr apart, followed 24 hr later by intravenous tail vein injection of myeloid BP-CML stem/progenitor cells. To abrogate antibody-mediated cell clearance, NSG were injected intraperitoneally with 200 mg of anti-CD122 antibody or IVIG (1 mg/gram body weight). Peripheral blood or bone marrow engraftment was monitored by blood sampling from 12 weeks onwards. Mice were culled for bone marrow harvesting between 16 and 22 weeks. Human myeloid (hCD45+CD33+CD19−) or B-lymphoid (hCD45+CD33−CD19+) engraftment was analysed by FACS and defined as ≥0.1% of live mononuclear cell (MNC) gate. Leukaemic engraftment was confirmed by karyotypic and BCR-ABL analysis.

### Flow Cytometric Analysis and Sorting

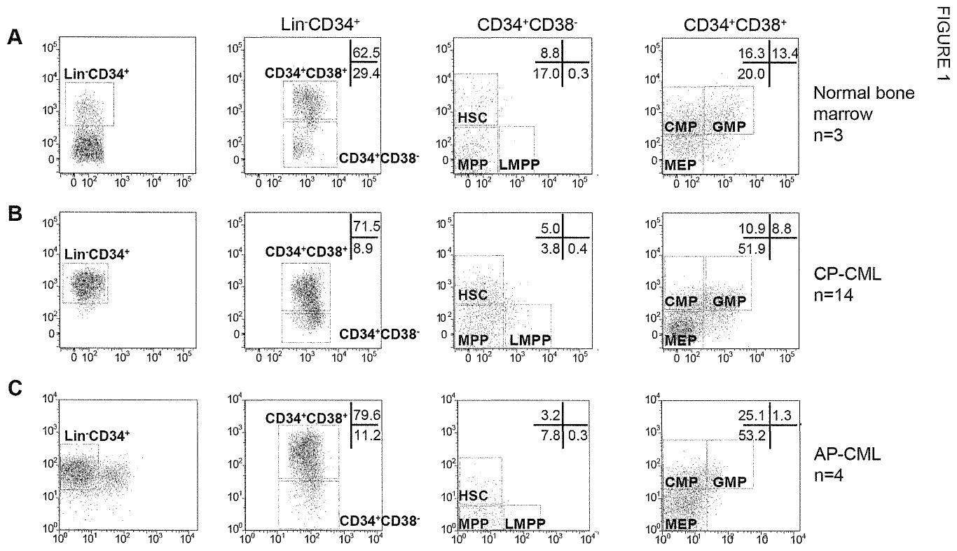

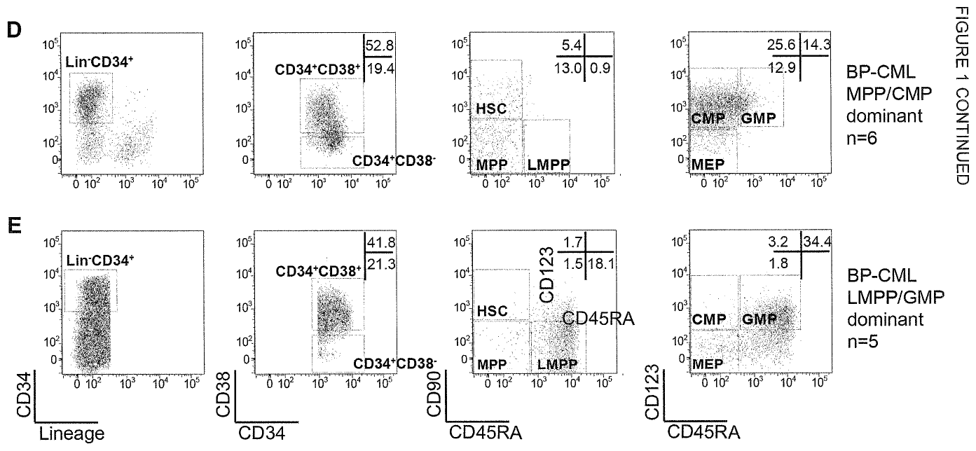

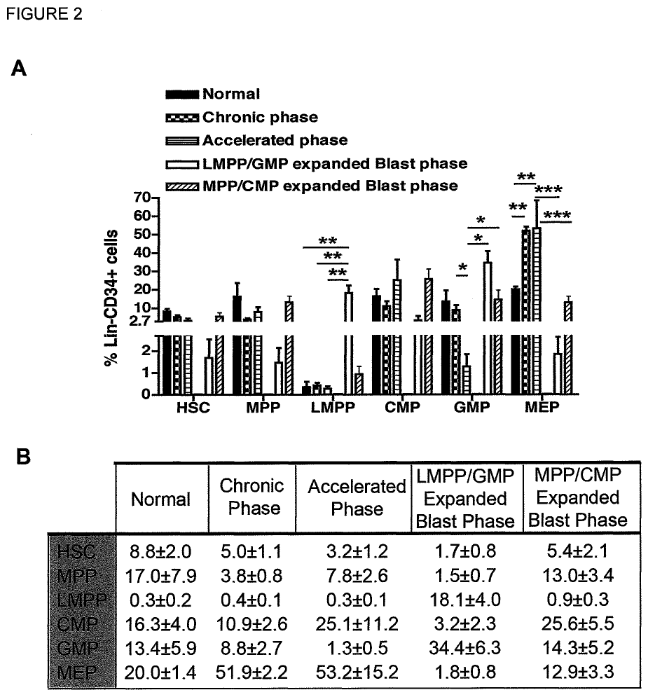

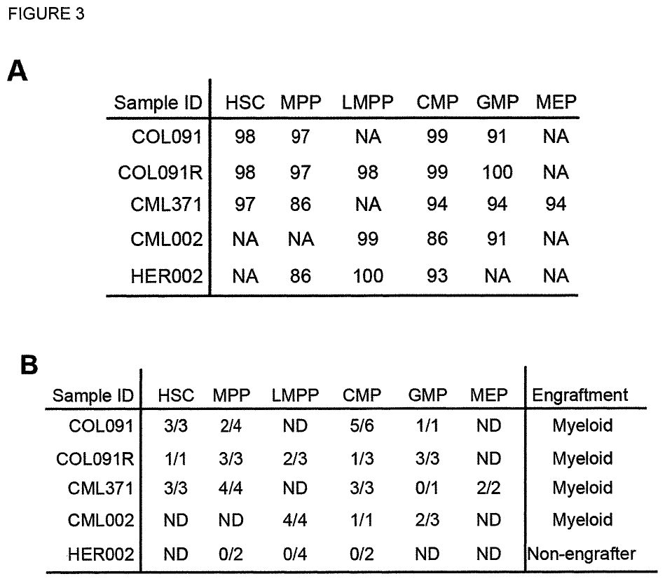

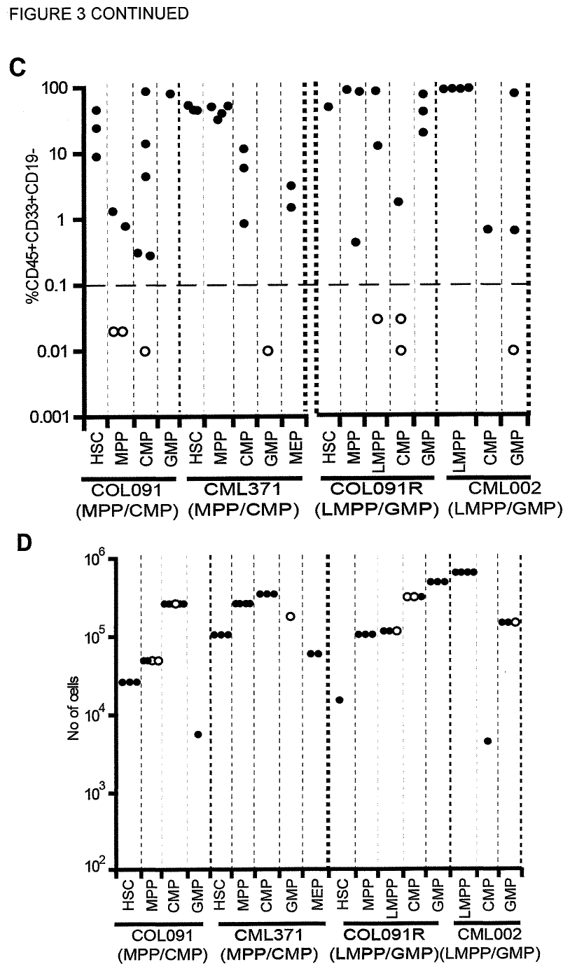

Different antibody panels were used for FACS purification of cells for injection into immunodeficient mice (FIGS. 2 and 3) and for quantitation of stem/progenitor sizes in primary human samples (FIG. 1). For FACS purification, additional antibodies (anti-CD2, anti-CD4, anti-CD8, anti-CD235a—glycophorin) against lymphoid (to avoid GvHD) and erythroid cells (to reduce ineffective cell number injected) but not myeloid cells (anti-CD14 and anti-CD16) were used. For FIG. 1, cells were stained with lineage cocktail-FITC (CD3 (MϕP9), 14 (3G8), 16 (NCAM16.2), 19 (SJ25C1), 20 (SK7), 56 (L27)) and CD34-PerCP (8G12), CD38-V450 (HIT2), CD45RA-APC H7 (HI100), CD90-PE Cy7 (5E10) and CD123-APC (7G3). All antibodies were from BD (Oxford, UK). In FIGS. 2 and 3, the following antibodies were used; lineage cocktail: anti-CD2 (RPA-2.10), CD3 (HIT3a), CD4 (RPA-T4), CD8 (RPA-T8), CD19 (HIB19), CD20 (2H7) and GPA (GA-R2) (all from e-Bioscience Hatfield UK). Following this, cells were stained with QDOT605 conjugated goat F(ab′) anti-mouse IgG (H+L, Invitrogen, Paisley UK). Cells were then washed and subsequently stained with FITC-conjugated anti-CD38 (HIT2, e-Bioscience), PE-conjugated anti-CD45RA (H1100, e-Bioscience), PerCP-conjugated anti-CD34 (581, BioLegend London UK), PECy7-conjugated anti-CD123 (6H6, e-Bioscience) and biotin-conjugated anti-CD90 (5E10, e-Bioscience). Finally cells were stained with a streptavidin-conjugated APCeFluor 780 (e-Bioscience). All samples were double sorted where cell numbers permitted (95% of sorts).

### FISH Analysis

FACS-sorted cells were incubated at 37° C. for 15 minutes in a hypotonic solution (0.075M KCl). Cells were then centrifuged at 1500 rpm for 5 minutes and resuspended in fixative (3:1 methanol: acetic acid), added in a dropwise manner whilst continuously vortexing. Cells were incubated at room temperature for 5 minutes and centrifuged at 12000 rpm for 2 minutes. The cells were washed twice in fixative (12000 rpm for 2 minutes) before re-suspension in 1 ml fresh fixative. 3 μl of fixed cell suspension was dropped onto a glass slide, air-dried and cell density checked using a phase contrast microscope. Probe mixes were prepared according to manufacturer's instructions and 2 μl added and covered with a coverslip sealed with rubber solution. The slide was placed in a hybridization chamber, heated to 75° C. for 5 minutes and then 37° C. overnight. Cover slips were removed and slides washed in a 0.4×SSC/3% NP40 wash buffer at 72° C. for two minutes and then a 2×SSC/1% NP40 wash buffer at room temperature for two minutes. DAPI mounting medium (Vector Laboratories, Peterborough UK) was applied to the slide, a coverslip attached and the slide analysed using a Zeiss Axio Imager Z2 and Cytovision software from Leica Biosystems. For multi-probe studies, probes to BCR-ABL fusion and deletions of p53 and iso(17)q were custom designed and manufactured (Empire Genomics) with BCR fluorescently labelled in green, ABL in Texas Red, TP53 in Gold and MPO (iso17q) in Aqua. All probes were used following the manufacturer's instructions. Standard BCRABL pattern is R1G1F2. We also observed 2 patterns of atypical BCR-ABL profiles: R1G1F3 and R1G1F1.

Immunophenotypic Analysis of CML Progression from CP to Myeloid BP

We first compared the size of different immunophenotypic haematopoietic stem and progenitor (HSPC) compartments: HSC (Lin-CD34+CD38-CD90+CD45RA−), MPP (Lin-CD34+CD38−CD90−CD45RA−), LMPP (Lin-CD34+CD38−CD90−CD45RA+), CMP (Lin-CD34+CD38+CD45RA−CD123+), GMP (Lin-CD34+CD38+CD45RA+CD123+) and megakaryocyte-erythroid progenitor (MEP; Lin-CD34+CD38+CD45RA−CD123−) in normal, CP-, accelerated phase (AP)- and myeloid BP-CML (FIG. 1A-E). When compared to normal, CP- and AP-CML (FIG. 1A-C respectively), in myeloid BPCML there are two distinct immunophenotypic patterns: a dominant MPP-like/CMPlike phenotype (FIG. 1D) and a dominant LMPP-like/GMP-like phenotype (FIG. 1E). This results in striking differences in the sizes of cellular sub-compartments within the Lin-CD34+ population between normal, CP-, AP- and myeloid BP-CML (FIG. 2A-B). LMPP-like cells were <0.5% of Lin-CD34+ in normal, CP- and AP-CML, but significantly increased with progression to LMPP-like/GMP-like BP-CML (18.1%, p<0.01). GMP-like cells decreased as disease progressed from normal and CP- to AP-CML (p<0.05), but significantly increased on progression to LMPP-like/GMP-like BP-CML (p<0.05). The MEP fraction was significantly expanded in CP and AP-CML (p<0.01), but significantly decreased on progression to BP-CML (p<0.001) (FIG. 2A-B). In contrast the size of the immunophenotypic HSC compartment remained relatively constant despite disease progression. In myeloid BP samples, with what we have termed an MPP-like/CMP-like profile, the overall percentage of CMP-like and MPP-like cells was not significantly increased.

In summary, we demonstrate dynamic changes in size of the different immunophenotypic HSPC-like compartments with disease progression in CML and heterogeneity of HSPC-like populations in myeloid BP-CML. As a next step we proceeded to functionally characterize the different HSPC-like compartments in myeloid BP-CML.

### LSC Function in Myeloid BP-CML

To identify which cell compartments contain leukaemia-propagating function, we purified HSPC-like populations from 5 BP-CML patient samples (COL091, COL091R CML371, CML002 and HER002) (FIG. 3A). For patient COL091 two samples were tested, the initial myeloid BP diagnostic sample and a follow-up sample at relapse (COL091R), 9 months after therapy with palliative oral 6-mercaptopurine chemotherapy. Samples were tested for engraftment in primary immunodeficient murine recipients (FIG. 3B). Human myeloid-only leukemic engraftment was detected in 4 of 5 samples (FIG. 3C) by immunophenotype and FISH analysis for leukaemia-associated mutations (see below).

Two engrafting samples had large MPP-like/CMP-like compartments (COL091, CML371). In samples with expanded MPP-like/CMP-like populations, HSC-like, MPP-like, CMP-like and MEP-like populations reproducibly engrafted.

Two engrafting samples had expansion of LMPP-like/GMP-like compartments (COL091R, CML002). In both samples, all HSPC-like populations that could be purified engrafted in primary recipient mice (FIG. 3C). Since varying numbers of cells were available and thus injected from each immunophenotypic compartment we considered whether failure to engraft primary recipients could be explained by injection of low cell numbers. In all 4 samples there were examples of engrafting populations (black dots) that had been injected at lower cell numbers compared to non-engrafting populations (white-centred dots) (FIG. 3D). Failure to engraft may have occurred either because: (i) LSC frequency was low in non-engrafting populations; cell numbers did not permit establishing LSC frequencies by limiting dilution analysis, (ii) cell populations were too small to purify (e.g. MEP-like compartment in COL091); (iii) we had insufficient cell numbers to inject into several mice (e.g. GMP-like compartment in CML371).

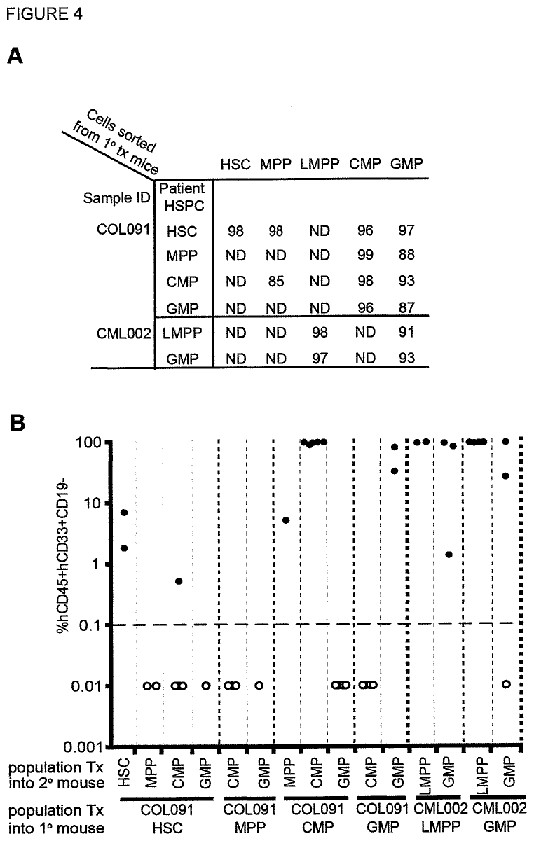

For 2 samples (COL091 and CML002), we purified cell populations from primary engrafted subpopulations (FIG. 4A) and analysed engraftment in secondary recipient mice (FIG. 4B). There were two aims of the experiment. First, we wanted to establish if patient HSPC populations could serially engraft leukaemia. For COL091, HSC-like, CMP-like and GMP-like populations serially propagated leukaemia (i.e. had LSC function). For sample CML002, both the LMPP- and GMP-like populations from the patient serially engrafted. Collectively, these data demonstrate multiple LSC populations in myeloid BP-CML.

Second, we asked if the leukemic HSPC populations were organized in a hierarchical manner akin to the hierarchy seen in normal haemopoiesis. In COL091, the HSC-like population from the patient generated an HSC-like population and downstream progenitor populations (FIG. 4A and B) but only the HSC-like and CMP-like populations from the primary mice engrafted secondary mice. Furthermore, when the CMP-like population from the patient was injected into primary mice, it generated an MPP-like population in addition to CMP-like and GMP-like progenitor-like populations. Both MPP-like and CMP-like populations from primary mice were able to engraft secondary recipients. However, although GMP-like cells from the patient generated CMP- and GMP-like populations, only the GMP-like cells from primary mice were able to engraft secondary mice. From patient CML002 both LMPP-like and GMP-like cells generated both LMPP-like and GMP-like populations in primary mice that could be purified and that were transplantable in secondary recipients. Based on these immunophenotypic analyses of engrafting populations, one interpretation of the data is that the leukaemia is not hierarchically organized.

### Analysis of Clonal Evolution in HSPC Populations in BP-CML

Clonal evolution, often associated with acquisition of additional cytogenetic abnormalities (ACAs) beyond t(9:22), is a marker of disease progression to AP- and BP-CML. Thus, we wanted to understand in which cell compartments ACAs were present in BP-CML by identifying clonal structures in myeloid BP-CML patients. Furthermore, given the multiple LSC populations in BP-CML, we asked if there was a correlation between LSC function and cytogenetic heterogeneity. Finally, we asked how accurately clonal structure in patients was captured in the experimental immunodeficient mouse model.

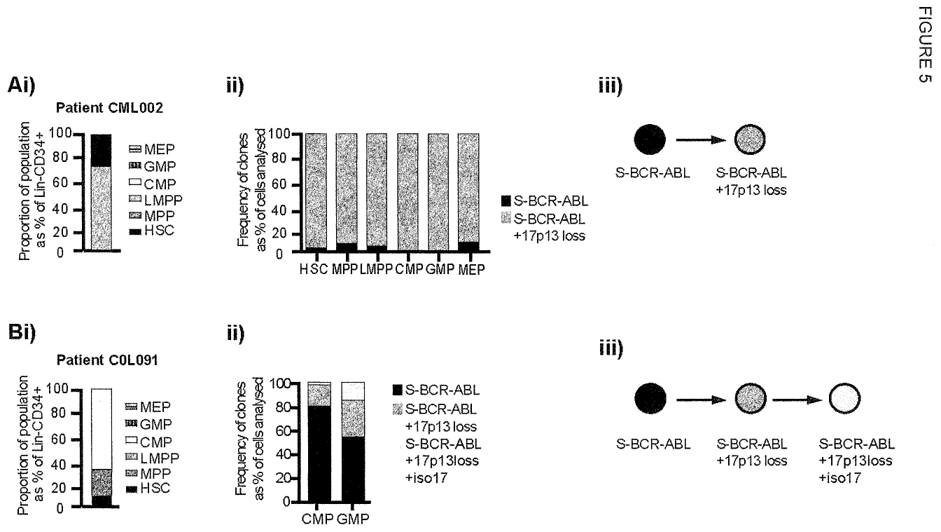

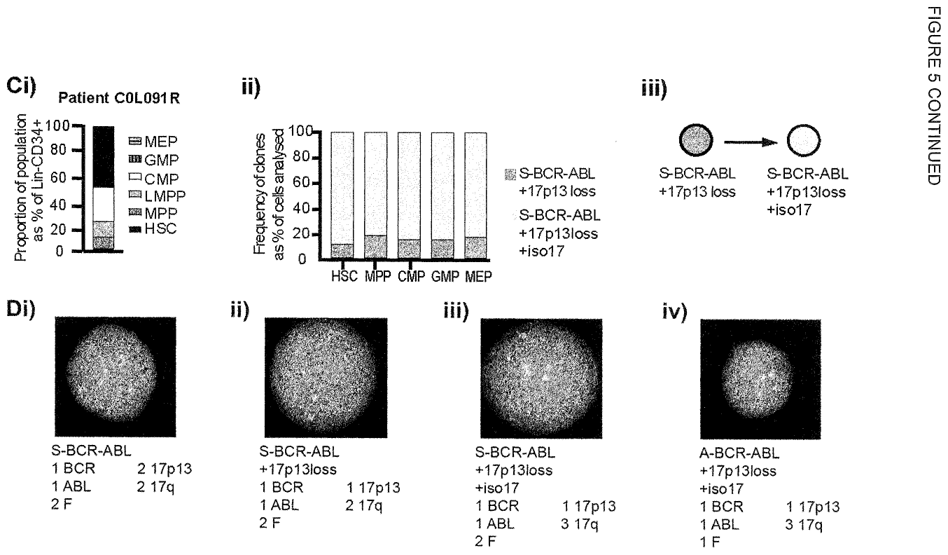

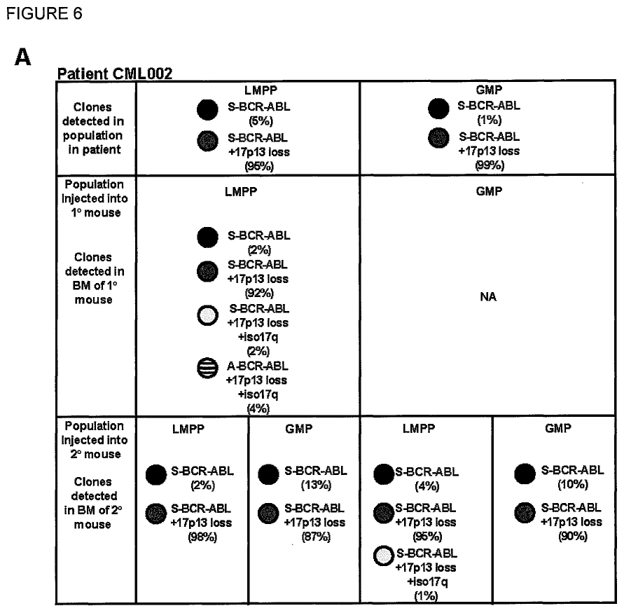

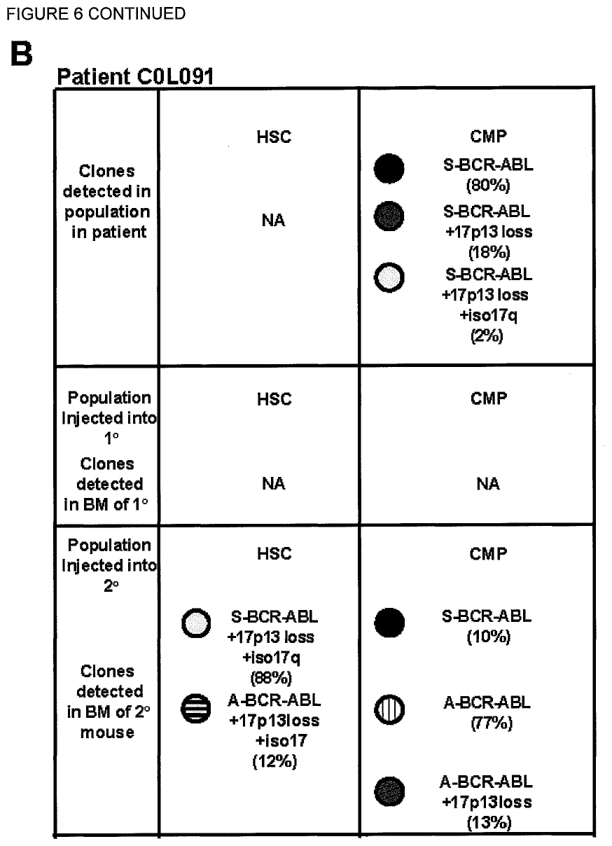

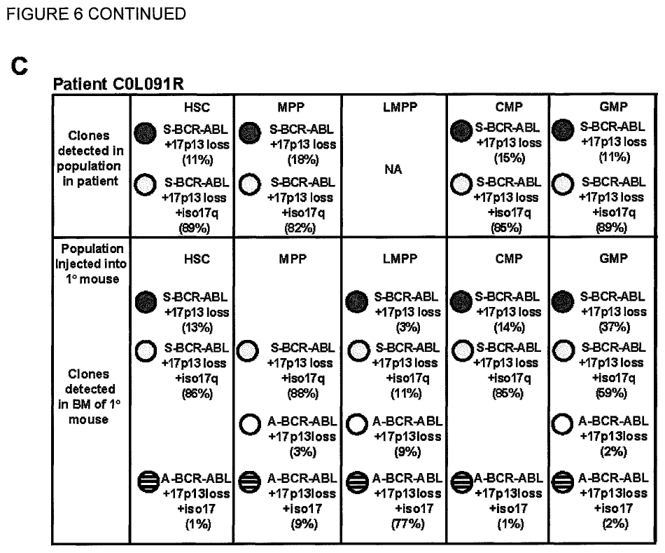

Karyotypic analysis of patient cells identified abnormalities in addition to t(9:22) in 4 patient samples (COL091, COL091R, CML371 and CML002) (Table 1). In ¾ cases (COL91, COL091R and CML002) we detected the ACAs by multicolor FISH at a level of >5%. Thus, we examined clonal structure in these cases in all available HSPC-like cell subpopulations from patient samples (FIG. 5A-C i-iii) and engrafted mice (FIG. 6A-C). For some subpopulations, and in some engrafted mice, insufficient cells were available for analysis.

Table 1 shows a) patient characteristics, and b) detailed cytogenetic analysis for the indicated patients obtained at diagnosis of BP-CML.
a)

| | | | | Disease |
|---|---|---|---|---|
| UPN | Age | Sex | WCC (×10⁹/L) | phase |
| CML264 | 61 | M | NK | Chronic |
| CML194 | 40 | F | 150 | Chronic |
| CML109 | 56 | F | NK | Chronic |
| CML259 | NK | NK | NK | Chronic |
| CML110 | 46 | F | NK | Chronic |
| CML111 | 35 | F | NK | Chronic |
| CML373 | 42 | F | 382 | Chronic |
| CML339 | 26 | F | 295 | Chronic |
| CML294 | 38 | F | 294 | Chronic |
| CML273 | 50 | F | 308 | Chronic |
| CML340 | NK | NK | NK | Chronic |
| CML341 | 48 | M | 231 | Chronic |
| CML332 | 54 | F | 132 | Chronic |
| CML343 | 39 | M | 48 | Chronic |
| CML347 | 27 | F | 386 | Chronic |
| CML112 | 32 | M | >100 | Accelerated |
| CML185 | 46 | M | >100 | Accelerates |
| CML239 | 62 | M | NK | Accelerated |
| CML415 | 71 | F | 135 | Accelarated |
| CML001 | NK | F | NK | Myeloid Blast |
| CML002 | 65 | M | NK | Myeloid Blasi |
| CML371 | 40 | M | 51 | Myeloid Blast |
| COL091 | 78 | M | NK | Myeloid Blast |
| COL091R | 78 | M | NK | Myeloid Blast |
| HER002 | 65 | F | NK | Myeloid Blast |
| LIV1723 | 36 | F | NK | Myelosd Blast |
| LIV1724 | 33 | M | NK | Myeloid Blast |
| LIV2616 | 43 | F | NK | Myeloid Blast |
| LIV2225 | 65 | F | NK | Myeloid Blast |
| LIV2397 | 42 | M | NK | Myeloid Blast |

| Patient | Karyotype |
|---|---|
| COL091 | 46, XY t(9; 22) (q34; q11), del(16) (q22q23) [8], |
| | 46, XY t(9; 22) (q34; q11), del(16) (q22q23), i(17) (?q10) [2] |
| COL091R | 46, XY t(9; 22) (q34; q11), del(16) (q22), i(17) (q10) [4] |
| CML371 | 46, XY t(9; 22) (q34; q11) [39]/ |
| | 46, idem, del(7)(p11)[5]/46, XY[6] |
| CML002 | 46, XY, t(9; 22) (q34; q11)[15] |
| | 46, idem, del(17)(p1?3) [15] |

Taking data from all three patient samples and engrafted mice together, the following points emerge. First, in all three patients the ACAs involved heterozygous loss of chromosome 17p (detected with a TP53 probe) and in 2/3 cases isochromosome 17%, consistent with previous data showing chromosome 17 aberrations are common in CML clonal evolution. Second, in all three cases, ACAs are detected in multiple immunophenotypic compartments regardless of which immunophenotypic compartments are expanded (FIG. 5A-Ci-iii). Third, in all three cases there were differences in clonal composition of cells in the patient and in the mice. For example, in the patient sample COL091, all three clones in patient cells had a standard BCRABL FISH signal (standard BCR-ABL, standard BCR-ABL plus 17p13 loss and standard BCR-ABL plus 17p13 loss with isochromosome 17q) (FIG. 51B). In contrast, in secondary transplanted mice, clones with an aberrant BCR-ABL signal were detected (FIG. 6B). In one of these mice the aberrant BCR-ABL clones comprised 90% of engrafted cells. In the relapsed sample COL091R, there was evidence of clones in the transplanted mice not detected in the patient sample (FIG. 6C).

We have fully characterized the leukaemia stem and progenitor cell populations in myeloid BP-CML. We demonstrated that in myeloid BP-CML the sizes of the immunophenotypic stem/progenitor compartments are heterogeneous and often show expanded progenitor populations. Serial transplantation indicated that LSCs can reside in any of the immunophenotypically-defined HSPC populations. Concordantly, we showed that ACAs are also present in the multiple immunophenotypic HSPC-like populations with LSC potential.

In summary, our examples conclusively showed that functional LSCs reside in multiple immunophenotypically distinct HSPC populations in myeloid BP-CML. This is associated with clonal evolution in all HSPC compartments, including the HSC-like populations.

### Example 2

### Identification of Differentially Expressed Genes in CMPs, MPPs, LMPPs, and GMPs Preparation RNA-Sequencing Libraries

˜100 highly purified haematopoietic stem and progenitor cell (HSPC) populations from bone marrow samples obtained from healthy and leukaemic donors were sorted directly into lysis buffer containing RNAse inhibitor (Clontech St Germain-en-Laye France) and were stored at −80° C. before further processing. cDNA synthesis was done with Smarter Ultra low input RNA kit vi (Clontech). Illumina libraries were generated using a Nextera XT DNA sample preparation kit and Index Kit (Illumina Chesterford UK). Library size and quality were checked using Agilent High-Sensitivity DNA chip with Agilent Bioanalyser (Agilent Technologies Stockport UK). The concentration of indexed libraries was determined using a Qubit High-Sensitivity DNA kit (Invitrogen Loughborough, UK). Libraries were pooled to a final concentration of 5-14 nM and were sequenced on an Illumina HiSeq 4000 paired-end 75-bp reads.

### Bioinformatic Analysis

For quality control, raw reads were assessed using Fastqc. Reads were trimmed using Trimmomatic for Nextera transposase sequences and over-represented sequences. Pseodoalignment was then carried out using Kallisto and coding genes were quantified. Generated counts tables were then processed in DESeq2. Lowly expressed genes (with fewer than 5 counts across all samples) were removed and VST normalisation used.

### MPP, CMP, GMP & LMPP Cell Surface Markers

To detect differentially expressed genes in a population a Wald test was used, comparing the population of interest to all other HSPC populations. Significantly upregulated genes were identified using a p-adjusted value of less than 0.05. These genes were filtered for cell surface markers using the human surfaceome described by Prof. Terence Rabbitts (http://www.imm.ox.ac.uk/complete-surfaceome-spreadheets).

Genes associated with the membrane and with a gold or silver ranking were taken forward as cell surface markers of interest.

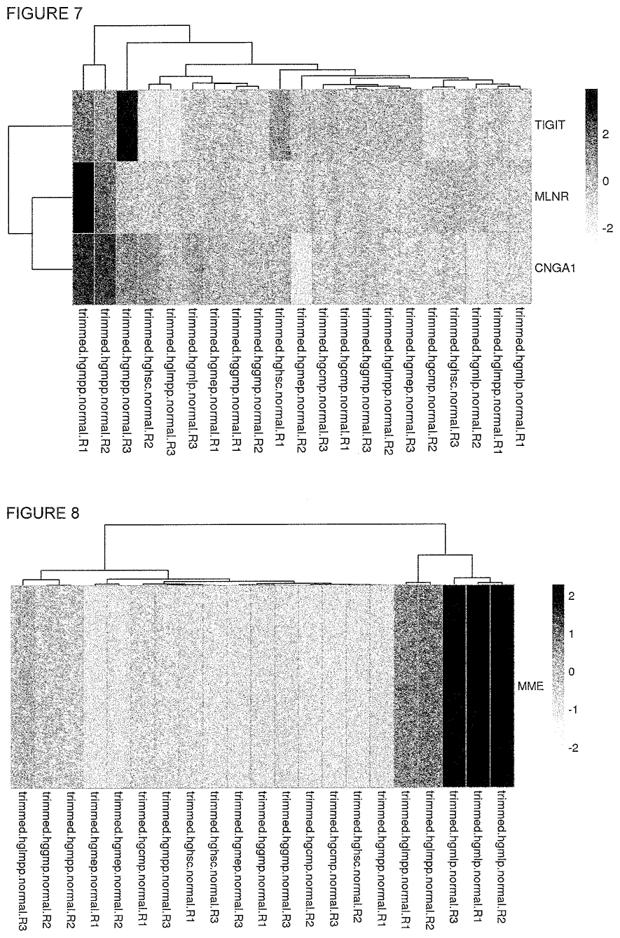

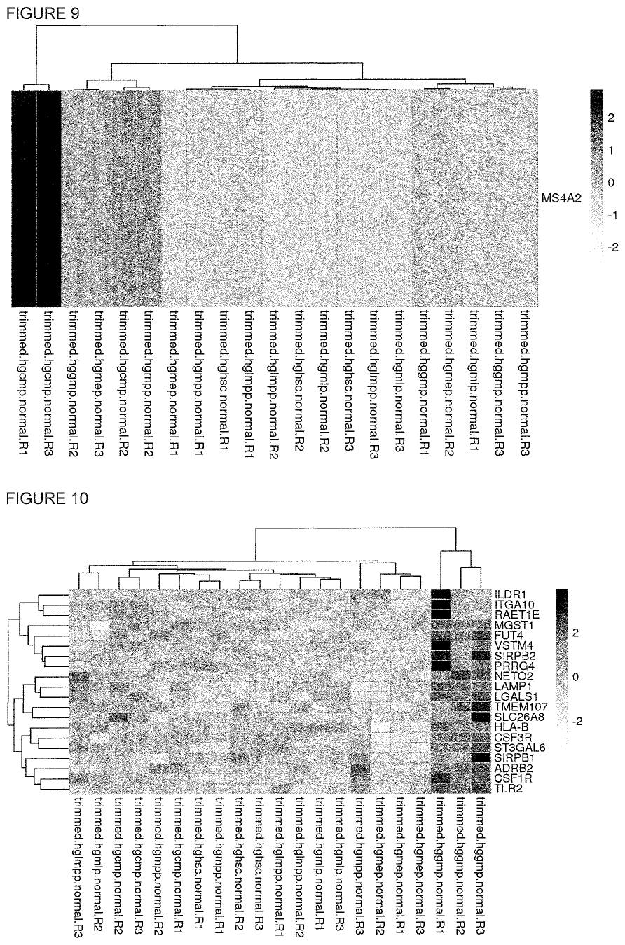

Expression data heat maps for each of the cell types compared to progenitor cells and HSCs are shown in FIGS. 7-10.

Results are indicated in the table below together with Log 2 fold change:

| | | Log2 Fold | P Adjusted |
|---|---|---|---|
| Cell Type | Gene | Change | Value < 0.05 |
| CMP | MS4A2 | 3.67732121 | 5.43E−07 |
| MPP | MLNR | 2.637313759 | 0.006348268 |
| | TIGIT | 2.618617415 | 0.013352476 |
| | CNGA1 | 2.365903308 | 0.023538679 |
| LMPP | MME | 2.996524856 | 9.71E−05 |
| GMP | SIRPB2 | 4.612995245 | 5.94E−12 |
| | PRRG4 | 2.157661029 | 1.24E−05 |
| | VSTM4 | 1.2431042 | 0.001337256 |
| | TMEM107 | 1.412986069 | 0.006136896 |
| | NETO2 | 1.978572835 | 0.006455439 |
| | CSF1R | 2.60699985 | 0.014350009 |
| | ADRB2 | 2.640170514 | 0.015261082 |
| | TLR2 | 2.57433613 | 0.015582145 |
| | FUT4 | 2.22526366 | 0.015675858 |
| | MGST1 | 1.809926672 | 0.02335672 |
| | CSF3R | 2.122162444 | 0.027773312 |
| | HLA-B | 1.189178651 | 0.028663457 |
| | ITGA10 | 1.570139712 | 0.030416667 |
| | SLC26A8 | 1.484785137 | 0.031823301 |
| | SIRPB1 | 1.656054158 | 0.034538793 |
| | RAET1E | 1.805128796 | 0.036933704 |
| | ST3GAL6 | 1.708125795 | 0.046048877 |
| | LAMP1 | 1.270162443 | 0.046552655 |
| | LGALS1 | 2.293728743 | 0.048539636 |
| | ILDR1 | 1.709756893 | 0.048539636 |

### LSC Cell Surface Markers

To detect differentially expressed genes in leukaemic samples a Wald test was used, comparing the normal (non-AML) LMPP and GMP with the leukaemic LMPP and GMP. Significantly upregulated genes were identified using a p-adjusted value of less than 0.05. These genes were filtered for cell surface markers as described above.

Results for the expression analysis in leukaemic LMPPs and GMPs are provided below:

| | Log2 Fold | P Adjusted |
|---|---|---|
| Gene | Change | Value < 0.05 |
| MME | −3.2649 | 6.53E−12 |
| IFITM1 | 2.791351 | 8.30E−07 |
| CMTM6 | 1.681428 | 1.95E−06 |
| CD55 | 1.885661 | 5.65E−06 |
| SLC35F5 | 1.654947 | 5.93E−06 |
| CNTNAP2 | −1.79013 | 9.33E−06 |
| PIGO | −2.12866 | 2.63E−05 |
| SHH | −3.1853 | 2.77E−05 |
| AQP11 | 3.059803 | 4.49E−05 |
| PCDHB9 | 1.283279 | 7.89E−05 |
| RHOA | 1.085482 | 0.000108 |
| TMEM231 | −1.79578 | 0.000119 |
| SAMD8 | 1.865806 | 0.00018 |
| ABCA13 | −2.17155 | 0.00019 |
| TAPT1 | 1.656336 | 0.000339 |
| NFASC | −0.94015 | 0.000529 |
| LEPROT | 1.581665 | 0.000531 |
| MCOLN2 | −1.57306 | 0.000556 |
| IL6ST | 2.314336 | 0.000567 |
| EMP3 | 1.664649 | 0.000988 |
| CD83 | 2.133856 | 0.001057 |
| LPAR6 | 2.683808 | 0.001064 |
| PIEZO2 | 2.461111 | 0.001372 |
| DERL1 | 0.958172 | 0.001398 |
| IL1RAP | 1.978059 | 0.001677 |
| LPAR4 | 2.597562 | 0.001794 |
| SERPINE2 | −2.04617 | 0.001929 |
| PKN2 | 1.063161 | 0.002199 |
| VAMP2 | 0.984519 | 0.002342 |
| TMCO3 | 1.125892 | 0.002733 |
| VAMP7 | 1.383903 | 0.002959 |
| PTPRC | 1.277407 | 0.003545 |
| TFRC | 1.521823 | 0.004709 |
| ILDR1 | −1.64782 | 0.005209 |
| PDIA3 | 1.248449 | 0.006379 |
| AIMP1 | 0.785261 | 0.006997 |
| GPR63 | −1.43823 | 0.00715 |
| CCR7 | −2.4021 | 0.007367 |
| ATG9B | −2.19614 | 0.008306 |
| SLC9B1 | 1.863979 | 0.00832 |
| CD99 | 1.247244 | 0.008614 |
| LRP1 | −1.27428 | 0.0096 |
| UBR4 | 0.602577 | 0.00976 |
| ATP6AP2 | 0.706512 | 0.011494 |
| TEX10 | 1.007287 | 0.011865 |
| CNGB1 | 1.14078 | 0.013097 |
| SPN | −0.92855 | 0.014206 |
| PILRB | −1.41094 | 0.014775 |
| JAM2 | −1.7028 | 0.014854 |
| PDGFA | 1.755207 | 0.014927 |
| CD46 | 0.761361 | 0.016004 |
| NDUFB1 | −0.82098 | 0.019129 |
| GYPE | −2.25737 | 0.020455 |
| SLC12A8 | 1.261887 | 0.022333 |
| SLC2A14 | 1.485765 | 0.023896 |
| RNF19B | 1.704733 | 0.023992 |
| SPCS1 | 0.715126 | 0.026233 |
| SLC35F6 | −1.26606 | 0.027562 |
| CD36 | 1.787822 | 0.027672 |
| ITM2B | 0.883228 | 0.030193 |
| GLG1 | −1.20068 | 0.031632 |
| SMIM24 | −0.96097 | 0.032408 |
| TMEM50A | 0.717638 | 0.033782 |
| HSPA5 | 1.404673 | 0.035466 |
| ITGAX | 1.336632 | 0.036142 |
| SLC24A2 | −0.76929 | 0.036328 |
| SLC2A3 | 1.571859 | 0.038215 |
| RAB11FIP3 | −1.54185 | 0.039326 |
| IL18R1 | 1.839034 | 0.04168 |
| CCDC47 | 1.107439 | 0.043935 |
| FZD4 | 0.893479 | 0.044161 |
| SHISA9 | 0.559108 | 0.044648 |
| SORCS1 | 1.904703 | 0.045901 |
| CLIC4 | 1.359124 | 0.046657 |

Fold changes of less than 0 indicate reduced expression (downregulation) when compared to the non-leukaemic (e.g. non-LSC) reference standard. Fold changes of more than 0 indicate increased expression (upregulation) when compared to the non-leukaemic (e.g. non-LSC) reference standard.

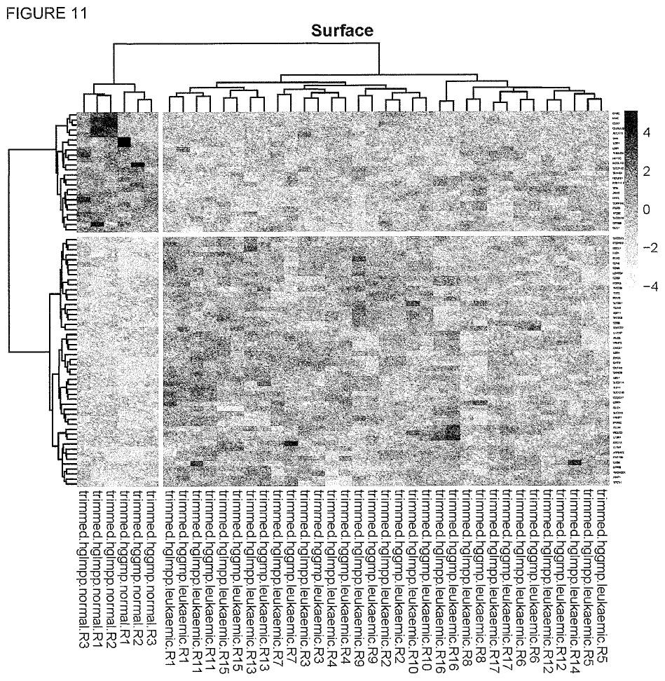

A heat map is provided in FIG. 11 showing expression data.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for identifying a leukaemic stem cell (LSC) in a sample, said method comprising:
a. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample;
b. comparing the detected expression of said one or more genes to expression of said one or more genes in a reference standard; and
c. identifying a an LSC in said sample based on said comparison.

2. A method for diagnosing myeloid leukaemia comprising detecting the presence or absence of a leukaemic stem cell (LSC) in a sample, said method comprising:
a. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample;
b. comparing the detected expression of said one or more genes to expression of said one or more genes in a reference standard; and
c. identifying the presence or absence of an LSC in said sample based on said comparison;
wherein myeloid leukaemia is diagnosed when said LSC is present in the sample; and
wherein myeloid leukaemia is not diagnosed when said LSC is absent from the sample.

3. A method for identifying a myeloid precursor cell in a sample, said method comprising:
a. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample;
b. comparing the detected expression of said one or more genes to expression of said one or more genes in a reference standard; and
c. identifying a myeloid precursor cell in said sample based on said comparison.

4. The method according to any one of the preceding claims, wherein the one or more genes are selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, and CLIC4.

5. The method according to any one of the preceding claims, wherein increased expression of: IFITM1, CMTM6, CD55, SLC35F5, AQP11, PCDHB9, RHOA, SAMD8, TAPT1, LEPROT, IL6ST, EMP3, CD83, LPAR6, PIEZO2, IL1RAP, LPAR4, PKN2, TMCO3, VAMP7, PTPRC, TFRC, PDIA3, SLC9B1, CD99, TEX10, CNGB1, PDGFA, SLC12A8, SLC2A14, RNF19B, CD36, HSPA5, ITGAX, SLC2A3, IL18R1, CCDC47, SORCS1, CLIC4, DERL1, VAMP2, AIMP1, UBR4, ATP6AP2, CD46, SPCS1, ITM2B, TMEM50A, FZD4, and/or SHISA9 in said sample when compared to a LMPP or GMPP reference standard identifies the presence of a LSC in said sample or the presence of myeloid leukaemia (e.g. AML).

6. The method according to any one of the preceding claims, wherein decreased expression of: MME, CNTNAP2, PIGO, SHH, TMEM231, ABCA13, NFASC, MCOLN2, SERPINE2, ILDR1, GPR63, CCR7, ATG9B, LRP1, SPN, PILRB, JAM2, NDUFB1, GYPE, SLC35F6, GLG1, SMIM24, SLC24A2 and/or RAB11FIP3 in said sample when compared to a LMPP or GMPP reference standard identifies the presence of a LSC in said sample or the presence of myeloid leukaemia (e.g. AML).

7. The method according to any one of the preceding claims, wherein the LSC is a LMPP or GMPP LSC.

8. The method according to any one of the preceding claims, wherein increased expression of:
i. MS4A2 in said sample when compared to the expression in a non-CMP reference standard identifies the presence of a CMP cell in said sample;
ii. MLNR, TIGIT and/or CNGA1 in said sample when compared to the expression in a non-MPP reference standard identifies the presence of a MPP cell in said sample;
iii. MME in said sample when compared to the expression in a non-LMPP reference standard identifies the presence of a LMPP cell in said sample; and
iv. SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and/or ILDR1 in said sample when compared to the expression in a non-GMP reference standard identifies the presence of a GMP cell in said sample.

9. The method according to any one of the preceding claims, wherein no difference in expression of:
i. MS4A2 in said sample when compared to the expression in a CMP myeloid precursor cell reference standard identifies the presence of a CMP cell in said sample;
ii. MLNR, TIGIT and/or CNGA1 in said sample when compared to the expression in a MPP myeloid precursor cell reference standard identifies the presence of a MPP cell in said sample;
iii. MME in said sample when compared to the expression in a LMPP myeloid precursor cell reference standard identifies the presence of a LMPP cell in said sample; and
iv. SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, LGALS1, and/or ILDR1 in said sample when compared to the expression in a GMP myeloid precursor cell reference standard identifies the presence of a GMP cell in said sample.

10. A method for diagnosing myeloid leukaemia, said method comprising:
a. detecting the concentration of a cell in a sample, wherein the cell is identified or detected according to the method of any one of the preceding claims;
b. comparing the concentration of the detected cell with the concentration of a cell with the same gene expression profile in a diagnostic reference standard; and
c. identifying the presence or absence of a concentration difference;
wherein said presence or absence of a concentration difference correlates with the presence or absence of myeloid leukaemia.

11. The method according to claim 10, wherein the diagnostic reference standard is a non-myeloid leukaemia reference standard.

12. The method according to claim 11, wherein: an increased concentration of a CMP cell in said sample when compared to the diagnostic reference standard indicates the presence of acute myeloid leukaemia, and wherein: no change in concentration (or a decreased concentration) of said CMP cell in said sample when compared to the diagnostic reference standard indicates the absence of acute myeloid leukaemia.

13. The method according to claim 11, wherein: a decreased concentration of a MPP cell in said sample when compared to the diagnostic reference standard indicates the presence of acute myeloid leukaemia, and wherein no change in concentration (or an increased concentration) of said MPP cell in said sample when compared to the diagnostic reference standard indicates the absence of acute myeloid leukaemia.

14. The method according to claim 11, wherein: an increased concentration of a LMPP cell in said sample when compared to the diagnostic reference standard indicates the presence of acute myeloid leukaemia; and wherein no change in concentration (or a decrease in concentration) of said LMPP cell in said sample when compared to the diagnostic reference standard indicates the absence of acute myeloid leukaemia.

15. The method according to claim 11, wherein: an increased concentration of a GMP cell in said sample when compared to the diagnostic reference standard indicates the presence of acute myeloid leukaemia, and wherein no change in concentration (or a decreased concentration) of said GMP cell in said sample when compared to the diagnostic reference standard indicates the absence of acute myeloid leukaemia.

16. The method according to claim 10, wherein the diagnostic reference standard is a chronic phase chronic myeloid leukaemia (CP-CML) or accelerated phase CML (AP-CML) reference standard.

17. The method according to claim 16, wherein: an increased concentration of a CMP cell in said sample when compared to the diagnostic reference standard indicates the presence of acute myeloid leukaemia (AML), and wherein a decreased concentration or no change in concentration of said CMP cell in said sample when compared to the diagnostic reference standard indicates the absence of AML.

18. The method according to claim 16, wherein: an increased concentration of a MPP cell in said sample when compared to the diagnostic reference standard indicates the presence of AML, and wherein a decreased concentration or no change in concentration of said MPP cell in said sample when compared to the diagnostic reference standard indicates the absence of AML.

19. The method according to claim 16, wherein: an increased concentration of a LMPP cell in said sample when compared to the diagnostic reference standard indicates the presence of acute myeloid leukaemia (AML), and wherein a decreased concentration or no change in concentration of said LMPP cell in said sample when compared to the diagnostic reference standard indicates the absence of AML.

20. The method according to claim 16, wherein: an increased concentration of a GMP cell in said sample when compared to the diagnostic reference standard indicates the presence of AML, and wherein a decreased concentration or no change in concentration of said GMP cell in said sample when compared to the diagnostic reference standard indicates the absence of AML.

21. The method according to claim 10, wherein the diagnostic reference standard is an AML reference standard.

22. The method according to claim 21, wherein: an increased concentration or no change in concentration of a CMP cell in said sample when compared to the diagnostic reference standard indicates the presence of acute myeloid leukaemia (AML), and wherein a decreased concentration of said CMP cell in said sample when compared to the diagnostic reference standard indicates the absence of AML.

23. The method according to claim 21, wherein: no change in concentration of a MPP cell in said sample when compared to the diagnostic reference standard indicates the presence of acute myeloid leukaemia (AML), and wherein an increased or decreased concentration of said MPP cell in said sample when compared to the diagnostic reference standard indicates the absence of AML.

24. The method according to claim 21, wherein: an increased concentration or no change in concentration of a LMPP cell in said sample when compared to the diagnostic reference standard indicates the presence of AML, and wherein a decreased concentration of said LMPP cell in said sample when compared to the diagnostic reference standard indicates the absence of AML.

25. The method according to claim 21, wherein: an increased concentration or no change in concentration of a GMP cell in said sample when compared to the diagnostic reference standard indicates the presence of AML, and wherein a decreased concentration of said GMP cell in said sample when compared to the diagnostic reference standard indicates the absence of AML.

26. The method according to any one of claims 10-25, wherein the AML is blast phase chronic myeloid leukaemia (BP-CML).

27. The method according to any one of claims 10-26 wherein the increase in concentration is an increase of at least 0.5%, 1%, 2%, 3% or 4%.

28. The method according to any one of claims 10-27 wherein the decrease in concentration is a decrease of at least 0.5%, 1%, 2%, 3% or 4%.

29. The method according to any one of claims 10-28 further comprising confirming that the cell in said sample is a leukaemic stem cell.

30. A method for diagnosing myeloid leukaemia, said method comprising:
detecting the presence or absence of a leukaemic stem cell (LSC) in a sample;
wherein the LSC comprises a cell surface polypeptide marker phenotype:
a. CD34⁺; CD45RA⁻; CD123⁺; and CD38⁺; or
b. CD34⁺, CD45RA⁻; CD90⁻; and CD38⁻;
wherein (+) indicates the presence and (−) indicates the absence of said cell surface polypeptide markers; and
wherein myeloid leukaemia is diagnosed when said LSC is present in the sample; and
wherein myeloid leukaemia is not diagnosed when said LSC is absent from the sample.

31. A method for diagnosing myeloid leukaemia, said method comprising:
a. detecting the concentration of a cell in a sample, wherein the cell comprises a cell surface polypeptide marker phenotype:
i. CD34⁺; CD45RA⁻; CD123⁺; and CD38⁺; or
ii. CD34⁺, CD45RA⁻; CD90⁻; and CD38⁻;
wherein (+) indicates the presence and (−) indicates the absence of said cell surface polypeptide markers;
b. comparing the concentration of the detected cell with the concentration of a cell with the same cell surface polypeptide marker phenotype in a diagnostic reference standard; and
c. identifying the presence or absence of a concentration difference;
wherein said presence or absence of a concentration difference correlates with the presence or absence of myeloid leukaemia.

32. The method according to claim 31, wherein the diagnostic reference standard is a non-myeloid leukaemia reference standard.

33. The method according to claim 32, wherein: an increased concentration of the cell comprising the cell surface polypeptide marker phenotype CD34⁺; CD45RA⁻; CD123⁺; and CD38⁺ in said sample when compared to the diagnostic reference standard indicates the presence of AML, and wherein: no change in concentration (or a decreased concentration) of said cell in said sample when compared to the diagnostic reference standard indicates the absence of AML.

34. The method according to claim 33, wherein: a decreased concentration of the cell comprising the cell surface polypeptide marker phenotype CD34⁺, CD45RA⁻; CD90⁻; and CD38⁻ in said sample when compared to the diagnostic reference standard indicates the presence of AML, and wherein no change in concentration (or an increased concentration) of said cell in said sample when compared to the diagnostic reference standard indicates the absence of AML.

35. The method according to claim 31, wherein the diagnostic reference standard is a chronic phase chronic myeloid leukaemia (CP-CML) or accelerated phase CML (AP-CML) reference standard.

36. The method according to claim 35, wherein: an increased concentration of the cell comprising the cell surface polypeptide marker phenotype CD34⁺; CD45RA⁻; CD123⁺; and CD38⁺ in said sample when compared to the diagnostic reference standard indicates the presence of AML, and wherein a decreased concentration (or no change in concentration) of said cell in said sample when compared to the diagnostic reference standard indicates the absence of AML.

37. The method according to claim 35, wherein: an increased concentration of the cell comprising the cell surface polypeptide marker phenotype CD34⁺, CD45RA⁻; CD90⁻; and CD38⁻ in said sample when compared to the diagnostic reference standard indicates the presence of AML, and wherein a decreased concentration (or no change in concentration) of said cell in said sample when compared to the diagnostic reference standard indicates the absence of AML.

38. The method according to claim 31, wherein the diagnostic reference standard is an AML reference standard.

39. The method according to claim 38, wherein: an increased concentration or no change in concentration of the cell comprising the cell surface polypeptide marker phenotype CD34⁺; CD45RA⁻; CD123⁺; and CD38⁺ in said sample when compared to the diagnostic reference standard indicates the presence of acute myeloid leukaemia (AML), and wherein a decreased concentration of said cell in said sample when compared to the diagnostic reference standard indicates the absence of AML.

40. The method according to claim 38, wherein: no change in concentration of the cell comprising the cell surface polypeptide marker phenotype CD34⁺, CD45RA⁻; CD90⁻; and CD38⁻ in said sample when compared to the diagnostic reference standard indicates the presence of AML, and wherein an increased or decreased concentration of said cell in said sample when compared to the diagnostic reference standard indicates the absence of AML.

41. The method according to any one of claims 31-40, wherein the AML is blast phase chronic myeloid leukaemia (BP-CML).

42. The method according to any one of claims 31-41 wherein the increase in concentration is an increase of at least 0.5%, 1%, 2%, 3% or 4%.

43. The method according to any one of claims 31-42 wherein the decrease in concentration is a decrease of at least 0.5%, 1%, 2%, 3% or 4%.

44. The method according to any one of claims 31-43 further comprising confirming that the cell in said sample is a leukaemic stem cell.

45. A method for determining prognosis in myeloid leukaemia comprising detecting the presence or absence of a leukaemic stem cell (LSC) in a sample, said method comprising:
a. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample;
b. comparing the detected expression of said one or more genes to expression of said one or more genes in a reference standard; and
c. identifying the presence or absence of an LSC in said sample based on said comparison;
wherein a poor prognosis is determined when said LSC is present in the sample; and
wherein a good prognosis is determined when said LSC is absent from the sample.

46. A method for determining prognosis in myeloid leukaemia, said method comprising:
detecting the presence or absence of a leukaemic stem cell (LSC) in a sample;
wherein the LSC comprises a cell surface polypeptide marker phenotype:
a. CD34⁺; CD45RA⁻; CD123⁺; and CD38⁺; or
b. CD34⁺, CD45RA⁻; CD90⁻; and CD38⁻;
wherein (+) indicates the presence and (−) indicates the absence of said cell surface polypeptide markers; and
wherein a poor prognosis is determined when said LSC is present in the sample; and
wherein a good prognosis is determined when said LSC is absent from the sample.

47. Use of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 for diagnosing myeloid leukaemia, or for determining prognosis in myeloid leukaemia, or for detecting an LSC, in vitro.

48. Use of a leukaemic stem cell for diagnosing myeloid leukaemia, or for determining prognosis in myeloid leukaemia, in vitro, said leukaemic stem cell comprising a cell surface polypeptide marker phenotype:
a. CD34⁺; CD45RA⁻; CD123⁺; and CD38⁺; or
b. CD34⁺, CD45RA⁻; CD90⁻; and CD38⁻;
wherein (+) indicates the presence and (−) indicates the absence of said cell surface polypeptide markers.

49. A method for identifying a therapeutic suitable for treating myeloid leukaemia, said method comprising:
a. contacting a sample with a therapeutic candidate, wherein said sample comprises LSCs;
b. incubating the sample and therapeutic candidate;
c. detecting the presence or absence of the LSCs; and
d. comparing the number of LSCs detected in c. with the number of LSCs detected in the isolated sample before step a.;
wherein the therapeutic candidate is identified as a therapeutic suitable for treating myeloid leukaemia when the relative number of LSCs is decreased after contact with the therapeutic candidate; or
wherein the therapeutic candidate is not identified as a therapeutic suitable for treating myeloid leukaemia when the relative number of LSCs is not decreased after contact with the therapeutic candidate; and
wherein the LSCs are detected by a method comprising:
i. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample;
ii. comparing the detected expression of said one or more genes to expression of said one or more genes in a reference standard; and
iii. identifying the presence or absence of an LSC in said sample based on said comparison.

50. A method for identifying a therapeutic suitable for treating myeloid leukaemia, said method comprising:
a. contacting a sample with a therapeutic candidate, wherein said sample comprises LSCs comprising a cell surface polypeptide marker phenotype:
i. CD34⁺; CD45RA⁻; CD123⁺; and CD38⁺; or
ii. CD34⁺, CD45RA⁻; CD90⁻; and CD38⁻;
wherein (+) indicates the presence and (−) indicates the absence of said cell surface polypeptide markers;
b. incubating the sample and therapeutic candidate;
c. detecting the presence or absence of the LSCs; and
d. comparing the number of LSCs detected in c. with the number of LSCs detected in the isolated sample before step a.;
wherein the therapeutic candidate is identified as a therapeutic suitable for treating myeloid leukaemia when the relative number of LSCs is decreased after contact with the therapeutic candidate; or
wherein the therapeutic candidate is not identified as a therapeutic suitable for treating myeloid leukaemia when the relative number of LSCs is not decreased after contact with the therapeutic candidate.

51. A method for monitoring efficacy of a therapeutic in treating myeloid leukaemia, said method comprising:
a. providing an isolated sample from a patient administered the therapeutic;
b. detecting the presence or absence of LSCs in said sample;
c. determining the relative number of said LSCs by comparing the number of LSCs detected in b. with the number of LSCs present in an isolated sample from the patient prior to administration of the therapeutic;
d. confirming efficacy of the therapeutic by identifying a relative decrease in the number of LSCs after administration with the therapeutic; or confirming the absence of efficacy of the therapeutic by identifying no decrease or an increase in the number of LSCs after administration therapeutic; and wherein the LSCs are detected by a method comprising:
i. detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1 in the sample;
ii. comparing the detected expression of said one or more genes to expression of said one or more genes in a reference standard; and
iii. identifying the presence or absence of an LSC in said sample based on said comparison.

52. A method for monitoring efficacy of a therapeutic in treating myeloid leukaemia, said method comprising:
a. providing an isolated sample from a patient administered the therapeutic;
b. detecting the presence or absence of LSCs in said sample, wherein said LSC comprises a cell surface polypeptide marker phenotype:
i. CD34⁺; CD45RA⁻; CD123⁺; and CD38⁺; or
ii. CD34⁺, CD45RA⁻; CD90⁻; and CD38⁻;
wherein (+) indicates the presence and (−) indicates the absence of said cell surface polypeptide markers;
c. determining the relative number of said LSCs by comparing the number of LSCs detected in b. with the number of LSCs present in an isolated sample from the patient prior to administration of the therapeutic;
d. confirming efficacy of the therapeutic by identifying a relative decrease in the number of LSCs after administration with the therapeutic; or confirming the absence of efficacy of the therapeutic by identifying no decrease or an increase in the number of LSCs after administration with the therapeutic.

53. The method or use according to any one of the preceding claims further comprising contacting the cell (e.g. the LSC) with detecting means, wherein said detecting means facilitates detection of one or more of the cell surface polypeptide markers.

54. The method or use according to claim 53, wherein the detecting means comprises one or more antibodies.

55. The method or use according to any one of the preceding claims, wherein the myeloid leukaemia is acute myeloid leukaemia or chronic myeloid leukaemia.

56. An isolated LSC, wherein said LSC comprises a cell surface polypeptide marker phenotype:
a. CD34⁺; CD45RA⁻; CD123⁺; and CD38⁺; or
b. CD34⁺, CD45RA⁻; CD90⁻; and CD38⁻;
wherein (+) indicates the presence and (−) indicates the absence of said cell surface polypeptide markers.

57. A composition comprising the isolated LSC according to claim 56.

58. The composition according to claim 57 further comprising detecting means, wherein said detecting means facilitates detection of one or more of the cell surface polypeptide markers.

59. The composition according to claim 58, wherein the detecting means comprises one or more antibodies.

60. A method of treating myeloid leukaemia comprising:
a. obtaining the results of the method according to any one of the preceding claims; and
b. treating myeloid leukaemia when said LSC is present.

61. The method of claim 60 further comprising:
c. detecting whether the LSC is present after treatment; and
d. re-administering said treatment when said LSC is present.

62. A method of treating myeloid leukaemia comprising:
a. obtaining the results of the method of any one of the preceding claims; and
b. treating myeloid leukaemia when myeloid leukaemia is diagnosed.

63. A method comprising detecting expression of one or more genes selected from:
MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1.

64. A method comprising detecting the presence or absence of a leukaemic stem cell (LSC) in a sample; wherein the LSC comprises a cell surface polypeptide marker phenotype:
a. CD34⁺; CD45RA⁻; CD123⁺; and CD38⁺; or
b. CD34⁺, CD45RA⁻; CD90⁻; and CD38⁻;
wherein (+) indicates the presence and (−) indicates the absence of said cell surface polypeptide markers.

65. A kit comprising means for detecting expression of one or more genes selected from: MME, IFITM1, CMTM6, CD55, SLC35F5, CNTNAP2, PIGO, SHH, AQP11, PCDHB9, RHOA, TMEM231, SAMD8, ABCA13, TAPT1, NFASC, LEPROT, MCOLN2, IL6ST, EMP3, CD83, LPAR6, PIEZO2, DERL1, IL1RAP, LPAR4, SERPINE2, PKN2, VAMP2, TMCO3, VAMP7, PTPRC, TFRC, ILDR1, PDIA3, AIMP1, GPR63, CCR7, ATG9B, SLC9B1, CD99, LRP1, UBR4, ATP6AP2, TEX10, CNGB1, SPN, PILRB, JAM2, PDGFA, CD46, NDUFB1, GYPE, SLC12A8, SLC2A14, RNF19B, SPCS1, SLC35F6, CD36, ITM2B, GLG1, SMIM24, TMEM50A, HSPA5, ITGAX, SLC24A2, SLC2A3, RAB11FIP3, IL18R1, CCDC47, FZD4, SHISA9, SORCS1, CLIC4, MS4A2, MLNR, TIGIT, CNGA1, SIRPB2, PRRG4, VSTM4, TMEM107, NETO2, CSF1R, ADRB2, TLR2, FUT4, MGST1, CSF3R, HLA-B, ITGA10, SLC26A8, SIRPB1, RAET1E, ST3GAL6, LAMP1, and LGALS1.

66. The kit according to claim 65, wherein the means for detecting expression comprise one or more primers, probes and/or antibodies.

67. The kit according to claim 65 or 66 further comprising instructions for use.
